(19) United States
(12) Patent Application Publication
(10) Pub. No.: US 2026/0011131 A1
(43) Pub. Date: Jan. 8, 2026

(54) AUTOMATIC CALIBRATION OF INTRAVASCULAR IMAGING CATHETER

(71) Applicant: Canon U.S.A., Inc., Melville, NY (US)

(72) Inventor: Lampros Athanasiou, Medford, MA (US)

(21) Appl. No.: 19/251,513

(22) Filed: Jun. 26, 2025

Related U.S. Application Data
(60) Provisional application No. 63/667,473, filed on Jul. 3, 2024.

(51) Int. Cl. G06V 10/82; A61B 5/00; G06T 7/60; G06T 7/73

(52) U.S. Cl. CPC G06V 10/82; A61B 5/0071; A61B 5/0073; A61B 5/0084; A61B 5/6852; G06T 7/60; G06T 7/73; A61B 2560/0223; G06T 2207/10064; G06T 2207/10101; G06T 2207/10132; G06T 2207/20; G06T 2207/30021

(57) ABSTRACT

One or more devices, systems, methods and storage mediums for performing calibration (ex vivo and/or in vivo), sheath detection, and/or performing intravascular imaging and/or optical coherence tomography (OCT) while detecting and/or characterizing one or more tissues are provided. Examples of applications include imaging, evaluating and diagnosing biological objects, such as, but not limited to, for Gastro-intestinal, cardio and/or ophthalmic applications, and being obtained via one or more optical instruments, such as, but not limited to, optical probes, catheters, capsules and needles (e.g., a biopsy needle). Preferably, the intravascular imaging devices, systems, methods, and storage mediums involve calibration and/or sheath detection feature(s) and/or include or involve a method, such as, but not limited to, using one or more images to detect and/or characterize the one or more tissues and/or to perform coregistration. Calibrated (ex vivo and/or in vivo) catheters or probes, devices, or systems may be used for improved imaging.

### CROSS-REFERENCE TO RELATED APPLICATION(S)

This application relates, and claims priority, to U.S. Patent Application Ser. No. 63/667,473, filed Jul. 3, 2024, the entire disclosure of which is incorporated by reference herein in its entirety.

### FIELD OF THE DISCLOSURE

This present disclosure generally relates to computer imaging, automatic calibration of intravascular imaging devices or catheters, and/or to the field of medical imaging, particularly to devices/apparatuses, systems, methods, and storage mediums for performing automatic calibration, tissue characterization, and/or imaging in one or more images and/or for using one or more imaging modalities, including but not limited to, angiography, Optical Coherence Tomography (OCT), Multi-modality OCT (MM-OCT), near-infrared fluorescence (NIRF), near-infrared auto-fluorescence (NIRAF), OCT-NIRAF, fluorescence, white light back-reflection, near-infrared spectroscopy (NIRS), robot imaging, robot imaging, continuum robot imaging, etc. Examples of OCT applications include imaging, evaluating, and diagnosing biological objects, including, but not limited to, for gastro-intestinal, cardio, and/or ophthalmic applications, and being obtained via one or more optical instruments, including, but not limited to, one or more optical probes, one or more catheters, one or more endoscopes, one or more capsules, and one or more needles (e.g., a biopsy needle). One or more devices, systems, methods and storage mediums for characterizing, examining and/or diagnosing, and/or measuring viscosity of, a sample or object (e.g., tissue, an organ, a portion of a patient, etc.) using an apparatus or system that uses and/or controls one or more imaging modalities and/or that uses artificial intelligence are discussed herein.

### BACKGROUND

Fiber optic catheters and endoscopes have been developed to access to internal organs. For example, in cardiology, OCT has been developed to see (e.g., capture and visualize) depth resolved images of vessels with a catheter. The catheter, which may include a sheath, a coil and an optical probe, may be navigated to a coronary artery.

Optical coherence tomography (OCT) is a technique for obtaining high-resolution cross-sectional images of tissues or materials, and OCT enables real time visualization. The aim of the OCT techniques is to measure the time delay of light by using an interference optical system or interferometry, such as via Fourier Transform or Michelson interferometers. A light from a light source delivers and splits into a reference arm and a sample (or measurement) arm with a splitter (e.g., a beamsplitter). A reference beam is reflected from a reference mirror (partially reflecting or other reflecting element) in the reference arm while a sample beam is reflected or scattered from a sample in the sample arm. Both beams combine (or are recombined) at the splitter and generate interference patterns. The output of the interferometer is detected with one or more detectors, such as, but not limited to, photodiodes or multi-array cameras, in one or more devices, such as, but not limited to, a spectrometer (e.g., a Fourier Transform infrared spectrometer). The interference patterns are generated when the path length of the sample arm matches that of the reference arm to within the coherence length of the light source. By evaluating the output beam, a spectrum of an input radiation may be derived as a function of frequency. The frequency of the interference patterns corresponds to the distance between the sample arm and the reference arm. The higher frequencies are, the greater are the differences in path length. Single mode fibers may be used for OCT optical probes, and double clad fibers may be used for fluorescence and/or spectroscopy. A multi-modality system, such as, but not limited to, an OCT, fluorescence, and/or spectroscopy system with an optical probe, is developed to obtain multiple information at the same time.

In order to acquire cross-sectional images of tubes and cavities such as vessels, and/or esophagus and nasal cavities, the optical probe is rotated with a fiber optic rotary joint (FORJ). A FORJ is the interface unit that operates to rotate one end of a fiber and/or an optical probe. In general, a free space beam coupler is assembled to separate a stationary fiber and a rotor fiber inside the FORJ. Besides, the optical probe may be simultaneously translated longitudinally during the rotation so that helical scanning pattern images are obtained. This translation is most commonly performed by pulling the tip of the probe back along a guidewire towards a proximal end and, therefore, referred to as a pullback.

Optical probes or catheters are part of or help to define the sample path and typically have a long size. Optical probes or catheters may stretch during use or when changing environmental material(s)/condition(s). As such, path matching between the reference path/arm and the sample path/arm may be performed manually to have the light interference correspond to a particular scanned region and to achieve OCT catheter calibration. However, manual calibration can be time consuming and lead to inadequate sheath-ring mark marching due to multiple rings appearing inside the sheath, environmental artifacts that appear in an OCT image, etc. The manual calibration process can be even more difficult when inexperienced users perform the process.

Even in cases with good path matching, an optical probe or catheter may require frequent re-calibration when the probe or catheter is inserted into the body since the probe or catheter may stretch during its use or can change due to environmental material(s)/condition(s) (e.g., condition/material change(s)).

Accordingly, it would be desirable to provide at least one imaging or optical apparatus/device, system, method, and storage medium that is able to automatically calibrate an optical probe or catheter without suffering from change(s) in environmental material(s)/condition(s), from stretching, and/or from sensitivities being affected by artifacts (so that detection of such artifacts is not required) and that is able to evaluate and characterize a target, sample, or object (e.g., a tissue, an organ, a part of a patient, a vessel, etc.). It also would be desirable to provide one or more probe/catheter/robot device techniques and/or structure for characterizing the target, sample, or object (e.g., a tissue, an organ, a part of a patient, a vessel, etc.) for use in at least one optical device, assembly, or system to achieve consistent, reliable detection, and/or characterization/imaging results at high efficiency and a reasonable cost of manufacture and maintenance.

### SUMMARY

Accordingly, it is a broad object of the present disclosure to provide imaging (e.g., OCT, NIRF, NIRAF, white light back-reflection, near-infrared spectroscopy (NIRS), robots, continuum robots, etc.) apparatuses, systems, methods and storage mediums for using and/or controlling multiple imaging modalities, that are able to perform automatic optical probe or catheter calibration without suffering from change(s) in environmental material(s)/condition(s) (e.g., blood versus air, in vivo versus ex vivo, or other condition/material change(s)), from stretching, and/or from sensitivities being affected by artifacts (so that detection of such artifacts is not required) and that are able to evaluate and characterize tissue in one or more images (e.g., intravascular images) with greater or maximum success and/or efficiency. It is also a broad object of the present disclosure to provide OCT devices, systems, methods, and storage mediums using an interference optical system, such as an interferometer (e.g., spectral-domain OCT (SD-OCT), swept-source OCT (SS-OCT), multimodal OCT (MM-OCT), Intravascular Ultrasound (IVUS), Near-Infrared Autofluorescence (NIRAF), Near-Infrared Spectroscopy (NIRS), Near-Infrared Fluorescence (NIRF), therapy modality using light, sound, or other source of radiation, etc.).

Further, it is a broad object of the present disclosure to provide one or more methods or techniques that operate to one or more of the following: (i) perform optical probe or catheter calibration automatically for, or associated with, an entire or whole pullback of catheter or probe for one or more intravascular images (such as, but not limited to, OCT images); (ii) reduce computational time to characterize the pullback and/or automatically calibrate a probe or catheter in one or more embodiments; (iii) automatically calibrate the probe or catheter in any environmental material/condition (e.g., blood versus air, in vivo versus ex vivo, or other condition/material change(s)) without having to detect detailed structure(s) (e.g., a sheath, an artifact, a whole catheter sheath, etc.); (iv) automatically calibrate a probe or catheter regardless of noise (e.g., even noisy probes or catheters may be automatically calibrated); (v) apply in vivo and ex vivo calibration feature(s) to the probe or catheter to ensure that the probe or catheter is calibrated for and compatible with any environment; (vi) apply one or more features of the present disclosure to achieve a probe or catheter that may be calibrated in different clinical calibration scenarios (e.g., hand touch, table and air calibration, etc.); (vii) use automatic skeleton sheath detection (e.g., by automatically selecting/detecting a skeleton of a sheath: (1) a detailed sheath detection is not required; (2) any possible artifact detection/removal that would be used or required for a detailed sheath detection is not required/needed (e.g., multiple ring formation); and/or (3) a shape (e.g., an oval shape or any other geometric shape) of the probe or catheter would not or may not affect a final result for the calibration); (viii) achieve detection of a blood position to automatically locate a position of a sheath, re-adjust an image, and re-calibrate a probe or catheter that may be affected by environmental changes/materials (e.g., in vivo calibration); and/or (ix) perform a more detailed tissue detection or characterization and/or imaging in one or more embodiments. One or more embodiments of the present disclosure overcome the aforementioned issue(s) of having probe(s) or catheter(s) be affected by change(s) in environmental material(s)/condition(s), by stretching, and/or by or from artifacts (e.g., where a probe or catheter may have sensitivities being affected by artifacts). Indeed, several methodologies of the present disclosure have been developed which use an apparatus, a system, a method, a storage medium, etc. that operate to achieve or do one or more of the aforementioned items: (i) through (ix).

As aforementioned, the fiber optic catheters and endoscopes of the present disclosure have been developed to access internal organs, tissues, or other targets, samples, or objects. For example in the cardiology, OCT (optical coherence tomography), white light back-reflection, NIRS (near infrared spectroscopy), and fluorescence technology have been developed to see structural and/or molecular images of vessels with a catheter. The catheter, which comprises a sheath and an optical probe in one or more embodiments, may be navigated to a target, sample, or object, such as, but not limited to, a coronary artery.

In order to acquire cross-sectional images of tubes and cavities, such as, but not limited to, vessels, an esophagus, and at least one nasal cavity, the optical probe may be rotated with a fiber optic rotary joint (FORJ). In addition, the optical probe may be simultaneously translated longitudinally during the rotation so that helical scanning pattern images are obtained. This translation may be performed by pulling the tip of the probe back towards a proximal end, and this translation is, therefore, referred to as a pullback. While particular tubes, cavities, or other targets, samples, or objects (e.g., coronary arteries) may be discussed herein, the targets, samples, or objects for which the features of the present disclosure may be used are not limited thereto. Additionally, while particular imaging modalities that may be used in combination are discussed herein (e.g., an intravascular OCT and fluorescence system), the imaging modalities that may be used with one or more features of the present disclosure are not limited thereto.

The present disclosure provides one or more features for use in one or more ex vivo and/or in vivo calibration apparatuses, devices, systems, probes, or one or more components thereof, and/or one or more methods for ex vivo and/or in vivo calibration and storage mediums for ex vivo and/or in vivo calibration. In one or more embodiments, artificial intelligence maybe used to perform ex vivo and/or in vivo calibration.

One or more apparatuses for calibrating a catheter or probe may include: one or more processors that operate to: obtain or receive one or more images or one or more A-line images; and automatically calibrate the catheter or probe using an ex vivo calibration before the catheter or probe is inserted into a target, sample, or object and an in vivo calibration after the catheter or probe is inserted into the target, sample, or object, wherein: for the ex vivo calibration, the one or more processors operate to detect one or more skeletons or portions of a sheath of the catheter or probe and determine whether the skeletons or portions of the sheath are in a target or set position, and for the in vivo calibration, the one or more processors operate to detect blood or a blood border position to automatically locate a position of the one or more skeletons or portions of the sheath and then adjust or re-adjust the image or the A-line image and calibrate or re-calibrate the catheter or probe to reduce or remove any effects caused by in vivo or environmental changes. The one or more processors may further operate to calibrate or re-calibrate the catheter or probe even in a case where high noise is present.

The catheter or probe may use one or more imaging modalities, where the one or more imaging modalities include one or more of the following: Optical Coherence Tomography (OCT), single modality OCT, multi-modality OCT, swept source OCT, optical frequency domain imaging (OFDI), intravascular ultrasound (IVUS), another lumen image(s) modality, near-infrared spectroscopy (NIRS), near-infrared fluorescence (NIRF), near-infrared auto-fluorescence (NIRAF), near-infrared, fluorescence, and/or an intravascular imaging modality.

In one or more embodiments, for the ex vivo calibration, the one or more processors may further operate to path match a reference path/arm of the catheter or probe and a sample path/arm of the catheter or probe by moving a delay line, or a motorized delay line, to change the reference path/arm so that a ring mark matches or substantially matches the sheath in at least one of the one or more images or A-line images. The one or more processors may further operate to one or more of the following: (i) crop an image of the one or more images or A-line images to an area of interest; (ii) filter the image; (iii) binarize the image; (iv) detect rectangles or shapes that include the skeletons or portions of binary objects of the sheath; (v) select the skeletons or portions having a height >h1 and <h2; (vi) find a plurality of differences of middle lines (RL) of the rectangles or shapes to a fixed line (GL), where the difference represents or corresponds to the rectangles or shapes of the binary objects of the sheath; (vii) determine whether a 1^{st} (RL−GL) difference of the plurality of differences is <4 and the rest of the (RL−GL) differences <than 21 through 25 or is between 25 and 21; and/or (viii) determine that the catheter or probe is ex vivo calibrated in a case where the 1^{st} (RL−GL) difference of the plurality of differences is <4 and the rest of the (RL−GL) differences <than 21 through 25 or is between 25 and 21 or, in a case where the catheter or probe is not yet ex vivo calibrated, then move the delay line, or the motorized delay line, to d to −d and repeat steps (i) through (vii) for a new image or A-line image that is acquired.

In one or more embodiments, the one or more processors may further operate to one or more of the following: (i) perform a pullback of the catheter or probe and/or obtain or receive the one or more images or the one or more A-line images of one or more imaging modalities from the pullback of the catheter or probe; and/or (ii) display the one or more images or the one or more A-line images on a display, store the one or more images or the one or more A-line images in a memory, or use the one or more images or the one or more A-line images to train one or more models or AI-networks to (a) perform the ex vivo calibration and/or the in vivo calibration and/or (b) automatically obtain the one or more images or the one or more A-line images of the one or more imaging modalities. In a case where the one or more processors have trained one or more models or AI-networks, one or more of the following may occur/exist: (i) the trained model is one or a combination of the following: a neural net model or neural network model, a deep convolutional neural network model, a recurrent neural network model with long short-term memory that can take temporal relationships across images or frames into account, a generative adversarial network (GAN) model, a consistent generative adversarial network (cGAN) model, a three cycle-consistent generative adversarial network (3cGAN) model, a model that can take temporal relationships across images or frames into account, a model that can take temporal relationships into account including tissue location(s) and/or calibration location(s) during pullback in a vessel and/or including tissue and/or calibration characterization data during pullback in a vessel, a model that can use prior knowledge about a procedure and incorporate the prior knowledge into the machine learning algorithm or a loss function, a model using feature pyramid(s) that can take different image resolutions into account, and/or a model using residual learning technique(s), a segmentation model, a segmentation model with post-processing, a model with pre-processing, a model with post-processing, a segmentation model with pre-processing, a deep learning or machine learning model, a semantic segmentation model or classification model, an object detection or regression model, an object detection or regression model with pre-processing or post-processing, a combination of a semantic segmentation model and an object detection or regression model, a model using repeated segmentation model technique(s), a model using feature pyramid(s), a genetic algorithm that operates to breed multiple models for improved performance, and/or a model using repeated object detection or regression model technique(s); and/or (ii) the one or more processors further operate to use one or more neural networks or convolutional neural networks to one or more of: load a trained model of images or A-line images; perform ex vivo and/or in vivo calibration on the catheter or probe; determine whether the ex vivo and/or in vivo calibration is/are accurate or correct; determine one or more of the characteristics of one or more objects, targets, or samples in the one or more images or the one or more A-line images; identify or detect the one or more objects, targets, or samples; overlay data on at least one of the one or more images or A-line images to show location(s) of intravascular image(s), the calibrated catheter or probe, and/or the objects, targets, or samples; incorporate image processing and machine learning (ML) or deep learning to automatically identify and locate portions or components of the catheter or probe and/or to perform ex vivo and/or in vivo calibration of the catheter or probe; incorporate image processing and machine learning (ML) or deep learning to automatically identify and locate the one or more objects, targets, or samples; display the results for the ex vivo and/or in vivo calibration, the identification/detection or characterization on a display; and/or acquire or receive image data during the pullback operation of the catheter or the optical probe.

In one or more embodiments, one or more of the following may occur/exist: (i) the one or more images or A-line images are in polar coordinates; (ii) the image of the one or more images or A-line images is binarized using bilateral filtering and/or non-linear smoothing; (iii) the image of the one or more images or A-line images is binarized using bilateral filtering and/or non-linear smoothing, wherein the bilateral filtering is performed using intensity differences of one or more pixels, which result in edge maintenance simultaneously with noise reduction; (iv) using one or more convolutions, a weighted average of neighborhood pixel intensities replace an intensity of a central pixel of a mask; (v) the one or more processors further operate to detect a border or borders of cross sections of the one or more images or the A-line images and/or to perform segmentation procedure(s) of the A-line cross-section(s); (vi) an image I of the one or more images or A-line images is binarized using bilateral filtering and/or non-linear smoothing, wherein a bilateral filter for the image I, and a window mask W is defined as:

$${I}^{′} ( x ) = \frac{1}{{W}_{p}} {\sum }_{{x}_{i} ∈ W} I ( {x}_{i} ) {f}_{r} ( ‖ I ( {x}_{i} ) - I ( x ) ‖ ) {g}_{s} ( ‖ {x}_{i} - x ‖ ) ,$$

having a normalization factor Wₚ: Wₚ=Σₓ_{ᵢ}_{∈w} fᵣ (∥(xᵢ)−I(x)∥)gₛ(∥xᵢ−x∥), where x are coordinates of the mask's central pixel and the parameters fᵣ and gₛ are a Gaussian kernel for smoothing differences in intensities and a spatial Gaussian kernel for smoothing differences in coordinates; and/or (vii) the one or more processors further operate to perform bilateral filtering for an image I, and a window mask W is defined as:

$${I}^{′} ( x ) = \frac{1}{{W}_{p}} {\sum }_{{x}_{i} ∈ W} I ( {x}_{i} ) {f}_{r} ( ‖ I ( {x}_{i} ) - I ( x ) ‖ ) {g}_{s} ( ‖ {x}_{i} - x ‖ ) ,$$

having a normalization factor Wₚ: Wₚ=Σₓ_{ᵢ}_{∈w} fᵣ(∥(xᵢ)−I(x)∥)gₛ(∥xᵢ−x∥), where x are the coordinates of a central pixel of the mask and the parameters fᵣ and gₛ are a Gaussian kernel for smoothing differences in intensities and a spatial Gaussian kernel for smoothing differences in coordinates. In one or more embodiments, one or more of the following may occur/exist: (i) the image, the image I, or the image I′ of the one or more images or A-line images is automatically thresholded using Otsu's thresholding method, and one or more binary objects are revealed or detected; (ii) the one or more processors further operate to apply a filtering technique or bilateral filtering and delete the catheter or probe from the one or more images or A-line images; (iii) the one or more processors further operate to apply Otsu's automatic thresholding; (iv) to automatically threshold cross sections of the one or more images or the A-line images or to automatically threshold the image, the image I, or the image I′ of the one or more images or A-line images, a threshold Thrₒₜₛᵤ for the image I′ is calculated using Otsu's thresholding method, and the pixels of the image I′ that are smaller than Thrₒₜₛᵤ are set to zero value, where the result is a binary image with a guide wire being represented by the zero objects and one or more binary objects are revealed or detected; (v) for all of the revealed or detected binary objects, rectangles or geometric shapes that include each revealed or detected binary object are calculated; (vi) for all of the revealed or detected binary objects, rectangles or geometric shapes that include each revealed or detected binary object are calculated, and for the rectangles or geometric shapes having a height between 3 (h1) and 100 (h2) pixels, the one or more processors further operate to calculate a middle line, RL, for each rectangle or geometric shape and to find an absolute difference of a respective middle line, RL, of each rectangle or geometric shape to a fixed line, GL; (vii) for all of the revealed or detected binary objects, rectangles or geometric shapes that include each revealed or detected binary object are calculated, and for the rectangles or geometric shapes having a height between 3 (h1) and 100 (h2) pixels, the one or more processors further operate to calculate a middle line, RL, for each rectangle or geometric shape and to find an absolute difference of a respective middle line, RL, of each rectangle or geometric shape to a fixed line, GL, where GL represents a line of a binary sheath of the rectangles or geometric shapes in the catheter or probe that is calibrated; and/or (viii) the one or more processors further operate to select the rectangles, geometric shapes, or boxes by selecting the skeletons or portions of the sheath of the catheter or probe having height >h1 and <h2. In one or more embodiments, one or more of the following may occur/exist: (i) for the in vivo calibration, the one or more processors further operate to perform imaging alignment by detecting the sheath of the probe or catheter by detecting the blood or the blood border position and using the sheath as a zero point for measurements to reduce or remove error(s) or the effects caused by changing environmental materials and/or condition(s); (ii) once the image or the catheter or probe is ex vivo calibrated, then the catheter or probe is inserted into the target, object, or sample; (iii) once the image or the catheter or probe is ex vivo calibrated and the catheter or probe is inserted into the target, object, or sample, then the delay line, or the motorized delay line, is not moved, and any calibration error is corrected by adjusting the image of the one or more images or A-line images; and/or (iv) the one or more processors further operate to one or more of the following: (1) acquire one image or A-line image of the one or more images or A-line images; (2) apply bilateral filtering and Otsu's thresholding method to one image or A-line image of the one or more images or A-line images; (3) detect a bottom line area of a biggest detected object or binary object, where the bottom line area corresponds to an outer sheath boundary for the catheter or the probe; and/or (4) shift one image or A-line image of the one or more images or A-line images such that a detected outer sheath boundary matches or substantially matches a zero point or position which corresponds to a ring mark or which corresponds to an outer surface of a sheath of the catheter or probe, where all distances are measured outward from the zero point or position.

In one or more embodiments, the apparatus may further include: an interference optical system that operates to: (i) receive and divide light from a light source into a first light with which the target, object, or sample is to be irradiated and which travels along a sample arm of the interference optical system and a second reference light, (ii) send the second reference light along a reference arm of the interference optical system for reflection off of a reference reflection of the interference optical system, and (iii) generate interference light by causing reflected or scattered light of the first light with which the target, object, or sample has been irradiated and the reflected second reference light to combine or recombine, and to interfere, with each other, the interference light generating one or more interference patterns; and one or more detectors that operate to continuously acquire the interference light and/or the one or more interference patterns to measure the interference or the one or more interference patterns between the combined or recombined light to obtain data for one or more imaging modalities, wherein: (i) a wavelength of the first light is shorter than a wavelength of the reflected or scattered light and/or the generated interference light, and/or (ii) the interference optical system or the catheter or probe includes a double clad fiber. In one or more embodiments, one or more of the following may occur/exist: (i) the light source that operates to produce the light; (ii) the light source that operates to produce the light, the light source producing the light to operate as an excitation laser or light having a wavelength of 400 nm-900 nm or 635 nm; and/or (iii) the light source that operates to produce the light, the light source producing the light as an excitation laser or light and coupling the excitation laser or light into the interference optical system, the optical probe, and/or one or more components of the optical probe and/or of the catheter. The one or more components of the catheter or probe may include or comprise a double clad fiber.

In one or more embodiments, an optical system may include: an interference optical system that operates to: (i) receive and divide light from a light source into a first light with which an object or sample is to be irradiated and which travels along a sample arm of the interference optical system and a second reference light, (ii) send the second reference light along a reference arm of the interference optical system for reflection off of a reference reflection of the interference optical system, and (iii) generate interference light by causing reflected or scattered light of the first light with which the object or sample has been irradiated and the reflected second reference light to combine or recombine, and to interfere, with each other, the interference light generating one or more interference patterns; one or more detectors that operate to continuously acquire the interference light and/or the one or more interference patterns to measure the interference or the one or more interference patterns between the combined or recombined light to obtain data for one or more imaging modalities, wherein a wavelength of the first light is shorter than a wavelength of the reflected or scattered light and/or the generated interference light. In one or more embodiments, the interference optical system or a probe of the interference optical system may include a double clad fiber.

In one or more embodiments, the one or more imaging modalities may include one or more of the following: Optical Coherence Tomography (OCT), single modality OCT, multi-modality OCT, swept source OCT, optical frequency domain imaging (OFDI), intravascular ultrasound (IVUS), another lumen image(s) modality, near-infrared spectroscopy (NIRS), near-infrared fluorescence (NIRF), near-infrared auto-fluorescence (NIRAF), near-infrared, fluorescence, and an intravascular imaging modality.

In one or more embodiments, an imaging apparatus may include: an interference optical system that operates to: (i) receive and divide light from a light source into a first light with which an object or sample is to be irradiated and which travels along a sample arm of the interference optical system and a second reference light, (ii) send the second reference light along a reference arm of the interference optical system for reflection off of a reference reflection of the interference optical system, and (iii) generate interference light by causing reflected or scattered light of the first light with which the object or sample has been irradiated and the reflected second reference light to combine or recombine, and to interfere, with each other, the interference light generating one or more interference patterns; and one or more detectors that operate to continuously acquire the interference light and/or the one or more interference patterns to measure the interference or the one or more interference patterns between the combined or recombined light to obtain data for one or more imaging modalities, wherein a wavelength of the first light is shorter than a wavelength of the reflected or scattered light and/or the generated interference light. In one or more embodiments, one or more of the following may occur: (i) the one or more detectors operate to continuously acquire the interference light and/or the one or more interference patterns in the interference optical system, optical probe, or catheter.

An imaging apparatus may include one or more processors that operate to perform a pullback of the optical probe or the catheter and/or obtain one or more images or frames of one or more imaging modalities from the pullback of the optical probe or the catheter. In one or more embodiments, the one or more imaging modalities may include one or more of the following: Optical Coherence Tomography (OCT), single modality OCT, multi-modality OCT, swept source OCT, optical frequency domain imaging (OFDI), intravascular ultrasound (IVUS), another lumen image(s) modality, near-infrared spectroscopy (NIRS), near-infrared fluorescence (NIRF), near-infrared auto-fluorescence (NIRAF), near-infrared, fluorescence, and/or an intravascular imaging modality. The one or more processors may further operate to display the one or more images on a display, store the one or more images in a memory, or use the one or more images to train one or more models or AI-networks to auto-detect or to perform automatic calibration and/or to automatically obtain one or more images of the one or more imaging modalities.

In a case where the interference optical system, the optical probe or catheter, or one or more components of the optical probe or catheter include or are attached to a double clad fiber, one or more of the following may exist: (i) the imaging apparatus further comprises one or more processors that operate to perform a pullback of the optical probe or the catheter and/or obtain one or more images or frames of one or more imaging modalities from the pullback of the optical probe or the catheter; (ii) the imaging apparatus further comprises: one or more processors that operate to perform a pullback of the optical probe or the catheter and/or obtain one or more images or frames of one or more imaging modalities from the pullback of the optical probe or the catheter, and the one or more processors further operate to automatically calibrate the interference optical system, the optical probe, or the catheter; and/or (iii) the interference optical system may further include an OCT sub-system and a sub-system for another imaging modality.

In one or more embodiments, a method for performing automatic calibration of an interference optical system, an optical probe, and/or one or more components of the optical probe and/or of a catheter of an imaging apparatus may include: performing an ex vivo calibration and an in vivo calibration. In one or more embodiments, a method for ex vivo and in vivo calibrating a catheter or probe of an apparatus may include: obtaining or receiving one or more images or one or more A-line images; automatically calibrating the catheter or probe using an ex vivo calibration, before the catheter or probe is inserted into a target, sample, or object, by detecting one or more skeletons or portions of a sheath of the catheter or probe and determining whether the skeletons or portions of the sheath are in a target or set position; and automatically calibrating the catheter or probe using an in vivo calibration, after the catheter or probe is inserted into the target, sample, or object, by detecting blood or a blood border position to automatically locate a position of the one or more skeletons or portions of the sheath and then adjusting or re-adjusting the image or the A-line image and calibrating or re-calibrating the catheter or probe to reduce or remove any effects caused by in vivo or environmental changes.

In one or more embodiments, a computer-readable storage medium may store at least one program that operates to cause one or more processors to execute a method for ex vivo and in vivo calibrating a catheter or probe of an apparatus. In one or more embodiments, a computer-readable storage medium storing at least one program that operates to cause one or more processors to execute a method for ex vivo and in vivo calibrating a catheter or probe of an apparatus, where the method may include: obtaining or receiving one or more images or one or more A-line images; automatically calibrating the catheter or probe using an ex vivo calibration, before the catheter or probe is inserted into a target, sample, or object, by detecting one or more skeletons or portions of a sheath of the catheter or probe and determining whether the skeletons or portions of the sheath are in a target or set position; and automatically calibrating the catheter or probe using an in vivo calibration, after the catheter or probe is inserted into the target, sample, or object, by detecting blood or a blood border position to automatically locate a position of the one or more skeletons or portions of the sheath and then adjusting or re-adjusting the image or the A-line image and calibrating or re-calibrating the catheter or probe to reduce or remove any effects caused by in vivo or environmental changes.

In one or more embodiments, a computer-readable storage medium storing at least one program that operates to cause one or more processors to execute a method for performing automatic calibration of an interference optical system, an optical probe, and/or one or more components of the optical probe and/or of a catheter of an imaging apparatus may be used where the method may include any feature discussed herein, including, but not limited to: using an excitation laser or light with a wavelength of a predetermined range or value on or in the interference optical system, the optical probe, and/or one or more components of the optical probe and/or of a catheter for a predetermined or set amount of time or more to perform the automatic calibration for the interference optical system, the optical probe, and/or the one or more components of the optical probe and/or of the catheter.

In one or more embodiments, the object, target, or sample may include one or more of the following: a vessel; a target, a specimen, or object; a tissue or tissues; a patient; an interference optical system; one or more optical probes; and/or one or more components of the one or more optical probes.

The one or more processors may further operate to perform the coregistration by co-registering an acquired or received angiography image or the constructed image (e.g., a carpet view) and an obtained one or more intravascular images, such as, but not limited to, OCT or IVUS images or frames.

In one or more embodiments, a loaded, trained model may be one or a combination of the following: a segmentation (classification) model, a segmentation model with pre-processing, a segmentation model with post-processing, an object detection (regression) model, an object detection model with pre-processing, an object detection model with post-processing, a combination of a segmentation (classification) model and an object detection (regression) model, a deep convolutional neural network model, a recurrent neural network model with long short-term memory that can take temporal relationships across images or frames into account, a model using feature pyramid(s) that can take different image resolutions into account, a genetic algorithm that operates to breed multiple models for improved performance (as compared with a case where the genetic algorithm is not used), a model using residual learning technique(s), and/or any other model discussed herein or known to those skilled in the art.

In one or more embodiments, the one or more processors may further operate to one or more of the following: (i) display an image for each of one or more imaging modalities on a display, wherein the one or more imaging modalities include one or more of the following: a tomography image; an Optical Coherence Tomography (OCT) image; a fluorescence image; a near-infrared auto-fluorescence (NIRAF) image; a near-infrared auto-fluorescence (NIRAF) image in a predetermined view, a carpet view, and/or an indicator view; a near-infrared fluorescence (NIRF) image, a near-infrared fluorescence (NIRF) image in a predetermined view, a carpet view, and/or an indicator view; a near-infrared spectroscopy (NIRS) image; a three-dimensional (3D) rendering; a 3D rendering of a vessel; a 3D rendering of a vessel in a half-pipe view or display; a 3D rendering of the object; a lumen profile; a lumen diameter display; a longitudinal view; computer tomography (CT); Magnetic Resonance Imaging (MRI); Intravascular Ultrasound (IVUS); an X-ray image or view; and an angiography view; and (ii) change or update the displays based on the tissue(s) or tissue characteristic(s) evaluation results, based on the automatic calibration results, and/or based on an updated location of the probe or catheter.

One or more embodiments of a non-transitory computer-readable storage medium storing at least one program for causing a computer to execute a method for training a model using artificial intelligence may be used with any method(s) discussed in the present disclosure, including but not limited to, one or more tissue(s) or tissue characteristic(s) evaluation/determination method(s), one or more automatic calibration characteristic(s) evaluation/determination and/or performance method(s).

One or more embodiments of any method discussed herein (e.g., training method(s), detecting method(s), imaging or visualization method(s), automatic calibration method(s), artificial intelligence method(s), etc.) may be used with any feature or features of the apparatuses, systems, other methods, storage mediums, or other structures discussed herein.

One or more of the artificial intelligence features discussed herein that may be used in one or more embodiments of the present disclosure, includes but is not limited to, using one or more of deep learning, a computer vision task, keypoint detection, a unique architecture of a model or models, a unique training process or algorithm, a unique optimization process or algorithm, input data preparation techniques, input mapping to the model, pre-processing, post-processing, and/or interpretation of the output data as substantially described herein or as shown in any one of the accompanying drawings.

In one or more embodiments, tissue(s) and or characteristic(s) of one or more tissues and/or automatic calibration may be evaluated and determined using an algorithm, such as, but not limited to, the Viterbi algorithm.

One or more embodiments of the present disclosure may track and/or calculate a tissue(s) or tissue characteristic(s) evaluation success rate and/or automatic calibration characteristic(s) evaluation success rate.

The following paragraphs describe certain explanatory embodiments. Other embodiments may include alternatives, equivalents, and modifications. Additionally, the explanatory embodiments may include several novel features, and a particular feature may not be essential to some embodiments of the devices, systems, and methods that are described herein.

According to other aspects of the present disclosure, one or more additional devices, one or more systems, one or more methods and one or more storage mediums using OCT and/or other imaging modality technique(s) to perform tissue characterization, to perform automatic calibration and/or calibration characterization, and to perform coregistration using artificial intelligence, including, but not limited to, deep or machine learning, using results of the tissue detection and/or tissue characterization and/or using results of the automatic calibration and/or calibration characterization for performing coregistration, etc., are discussed herein. Further features of the present disclosure will in part be understandable and will in part be apparent from the following description and with reference to the attached drawings.

In accordance with one or more embodiments of the present disclosure, apparatuses and systems, and methods and storage mediums for tissue detection and/or tissue characterization and/or for automatic calibration and/or calibration characterization in one or more images may operate to characterize biological objects, such as, but not limited to, blood, mucus, tissue (including different types of tissue), etc.

It should be noted that one or more embodiments of the tissue detection and/or characterization method(s) or feature(s) and/or one or more embodiments of the automatic calibration and/or calibration characterization method(s) or feature(s) of the present disclosure may be used in other imaging systems, apparatuses or devices, where images are formed from signal reflection and scattering within tissue sample(s) using a scanning probe. For example, IVUS images may be processed in addition to or instead of OCT images.

One or more embodiments of the present disclosure may be used in clinical application(s), such as, but not limited to, intervascular imaging, atherosclerotic plaque assessment, cardiac stent evaluation, balloon sinuplasty, sinus stenting, arthroscopy, ophthalmology, ear research, veterinary use and research, etc.

In accordance with at least another aspect of the present disclosure, one or more technique(s) discussed herein may be employed to reduce the cost of at least one of manufacture and maintenance of the one or more apparatuses, devices, systems and storage mediums by reducing or minimizing a number of optical components and by virtue of the efficient techniques to cut down cost of use/manufacture of such apparatuses, devices, systems and storage mediums.

According to other aspects of the present disclosure, one or more additional devices, one or more systems, one or more methods and one or more storage mediums using, or for use with, one or more tissue detection and/or tissue characterization techniques and/or one or more automatic calibration and/or calibration characterization techniques are discussed herein. Further features of the present disclosure will in part be understandable and will in part be apparent from the following description and with reference to the attached drawings.

### BRIEF DESCRIPTION OF THE DRAWINGS

For the purposes of illustrating various aspects of the disclosure, wherein like numerals indicate like elements, there are shown in the drawings simplified forms that may be employed, it being understood, however, that the disclosure is not limited by or to the precise arrangements and instrumentalities shown. To assist those of ordinary skill in the relevant art in making and using the subject matter hereof, reference is made to the appended drawings and figures, wherein:

FIG. 1A is a schematic diagram showing at least one embodiment of a system that may be used for performing one or multiple imaging modality viewing and control and/or for at least performing automatic calibration of an interference optical system, a probe, and/or a catheter and/or for tracking/evaluating calibration characteristic(s) in accordance with one or more aspects of the present disclosure;

FIG. 1B is a schematic diagram illustrating an imaging system for executing one or more steps to process image data and/or for at least performing automatic calibration of an interference optical system, a probe, and/or a catheter and/or for tracking/evaluating calibration characteristic(s) in accordance with one or more aspects of the present disclosure;

FIG. 2 is a diagram of at least one embodiment of a catheter that may be used with one or more embodiments for at least performing automatic calibration and/or tracking/evaluating calibration characteristic(s) in accordance with one or more aspects of the present disclosure;

FIG. 3 shows at least one embodiment of an ex vivo path matching process where the left image depicts a ring mark and a sheath not matching and the right image depicts an ex vivo calibrated catheter having the ring mark and sheath match in accordance with one or more aspects of the present disclosure;

FIG. 4 is a schematic diagram of at least one calibration algorithm that may be used in accordance with one or more aspects of the present disclosure;

FIG. 5 shows a flow diagram showing at least one method embodiment for performing automatic calibration for an interference optical system, at least one optical probe, or at least one catheter that may be used in accordance with one or more aspects of the present disclosure;

FIG. 6 is a schematic diagram of at least one embodiment example of an ex vivo calibration portion of a calibration algorithm that may be used in accordance with one or more aspects of the present disclosure;

FIGS. 7A-7E shows at least one embodiment example of one or more ex vivo calibration steps or portion of a calibration algorithm applied in a calibrated image in accordance with one or more aspects of the present disclosure;

FIG. 8A shows seventeen (17) image frames each having an area of interest for each first frame of the seventeen (17) pullbacks of Table 1 of the present disclosure where a long dashed or dotted frame represents that no calibration is detected by the ex vivo calibration algorithm and the short dotted frame represents that calibration was detected in accordance with one or more aspects of the present disclosure;

FIG. 8B shows at least one embodiment of A-line and cross-sectional images for each first frame of the calibrated stationary pullbacks of Table 1 where a long dashed or dotted frame represents that no calibration is detected by the ex vivo calibration algorithm and the short dotted frame represents that calibration was detected in accordance with one or more aspects of the present disclosure;

FIG. 9A shows at least one embodiment of an apparatus or system for utilizing one or more imaging modalities and/or artificial intelligence for performing imaging, automatic calibration, and/or co-registration in accordance with one or more aspects of the present disclosure;

FIG. 9B shows at least another embodiment of an imaging apparatus or system for utilizing one or more imaging modalities and artificial intelligence for performing imaging, automatic calibration, and/or co-registration in accordance with one or more aspects of the present disclosure;

FIG. 9C shows at least a further embodiment of an imaging (such as, but not limited to, OCT, OCT and NIRF/NIRAF, OCT and fluorescence, etc.) apparatus or system for utilizing one or more imaging modalities and artificial intelligence for performing imaging, automatic calibration, and/or co-registration in accordance with one or more aspects of the present disclosure;

FIG. 10 is a flow diagram showing a method of performing an imaging feature, function, or technique in accordance with one or more aspects of the present disclosure;

FIG. 11 shows a schematic diagram of an embodiment of a computer that may be used with one or more embodiments of an apparatus or system or one or more methods discussed herein in accordance with one or more aspects of the present disclosure;

FIG. 12 shows a schematic diagram of another embodiment of a computer that may be used with one or more embodiments of an imaging apparatus or system or methods discussed herein in accordance with one or more aspects of the present disclosure;

FIG. 13 shows a schematic diagram of at least an embodiment of a system using a computer or processor, a memory, a database, and input and output devices in accordance with one or more aspects of the present disclosure;

FIG. 14 shows a created architecture of or for a regression model(s) that may be used for imaging, tissue characterization, tissue identification, automatic calibration, performing co-registration, and/or any other technique discussed herein in accordance with one or more aspects of the present disclosure;

FIG. 15 shows a convolutional neural network architecture that may be used for imaging, automatic calibration, calibration characterization, tissue characterization, tissue detection, and/or any other technique discussed herein in accordance with one or more aspects of the present disclosure;

FIG. 16 shows a created architecture of or for a regression model(s) that may be used for imaging, automatic calibration, calibration characterization, tissue characterization, tissue detection, and/or any other technique discussed herein in accordance with one or more aspects of the present disclosure; and

FIG. 17 is a schematic diagram of or for a segmentation model(s) that may be used for imaging, automatic calibration, calibration characterization, tissue characterization, tissue detection, and/or any other technique discussed herein in accordance with one or more aspects of the present disclosure.

### DETAILED DESCRIPTION

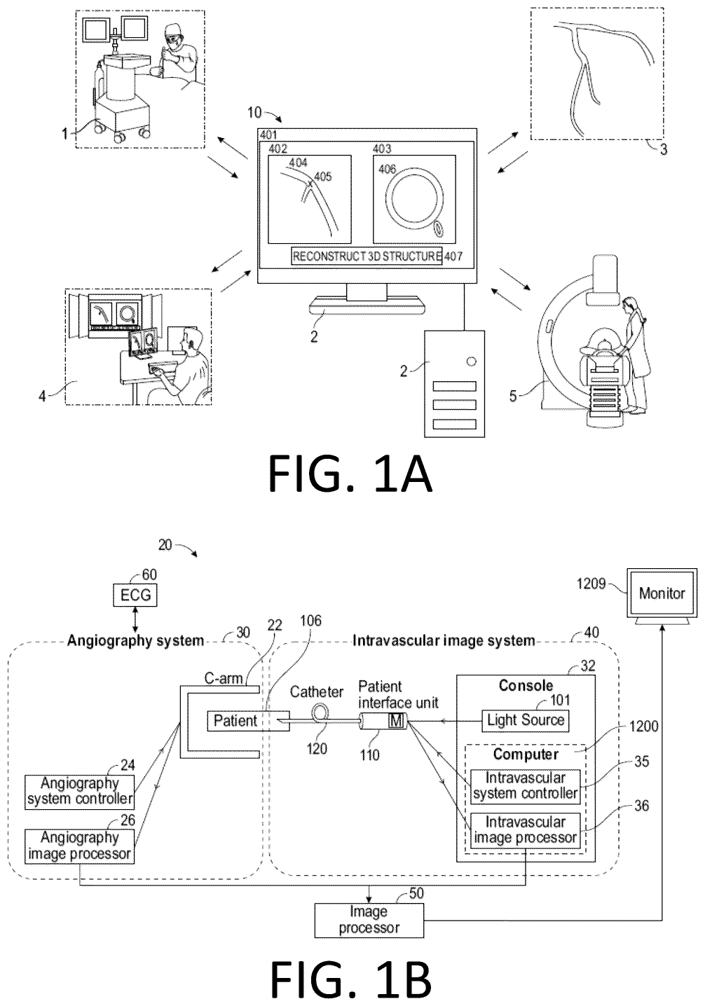

One or more devices, systems, methods and storage mediums for characterizing tissue, or an object, using one or more imaging techniques or modalities (such as, but not limited to, OCT, fluorescence, IVUS, MRI, CT, NIRF, NIRAF, NIRS, etc.), and using artificial intelligence for performing automatic calibration and/or evaluating calibration characteristics, detecting tissue types and/or characteristics, and/or performing coregistration are disclosed herein. Several embodiments of the present disclosure, which may be carried out by the one or more embodiments of an apparatus, system, method and/or computer-readable storage medium of the present disclosure are described diagrammatically and visually in at least FIGS. 1A through 17 and further discussed below.

One or more embodiments of the present disclosure provide at least one imaging or optical apparatus/device, system, method, and storage medium that may perform automatic calibration and/or evaluate/determine calibration characteristics.

One or more embodiments of the present disclosure provide at least one imaging or optical apparatus/device, system, method, and storage medium that may evaluate and characterize a target, sample, or object (e.g., a tissue, an organ, a part of a patient, a vessel, etc.). One or more embodiments of the present disclosure may also provide or use one or more probe/catheter/robot device techniques and/or structure for characterizing the target, sample, or object (e.g., a tissue, an organ, a part of a patient, a vessel, etc.) for use in at least one optical device, assembly, or system to achieve consistent, reliable detection, and/or characterization results at high efficiency and a reasonable cost of manufacture and maintenance.

One or more embodiments of the present disclosure provide imaging (e.g., OCT, NIRF, NIRAF, robots, continuum robots, etc.) apparatuses, systems, methods and storage mediums for using and/or controlling multiple imaging modalities, that may apply machine learning, especially deep learning, to perform automatic calibration and/or evaluate and characterize tissue in one or more images (e.g., intravascular images) with greater or maximum success. One or more embodiments of the present disclosure may operate to provide OCT devices, systems, methods, and storage mediums using an interference optical system, such as an interferometer (e.g., spectral-domain OCT (SD-OCT), swept-source OCT (SS-OCT), multimodal OCT (MM-OCT), Intravascular Ultrasound (IVUS), Near-Infrared Autofluorescence (NIRAF), Near-Infrared Spectroscopy (NIRS), Near-Infrared Fluorescence (NIRF), therapy modality using light, sound, or other source of radiation, etc.).

Accordingly, it is a broad object of the present disclosure to provide imaging (e.g., OCT, NIRF, NIRAF, white light back-reflection, near-infrared spectroscopy (NIRS), robots, continuum robots, etc.) apparatuses, systems, methods and storage mediums for using and/or controlling multiple imaging modalities, that are able to perform automatic optical probe or catheter calibration without suffering from change(s) in environmental material(s)/condition(s) (e.g., blood versus air, in vivo versus ex vivo, or other condition/material change(s), etc.), from stretching, and/or from sensitivities being affected by artifacts (so that detection of such artifacts is not required) and that are able to evaluate and characterize tissue in one or more images (e.g., intravascular images, images having different imaging modalities, images having one or more imaging modalities, etc.) with greater or maximum success and/or efficiency. It is also a broad object of the present disclosure to provide OCT devices, systems, methods, and storage mediums using an interference optical system, such as an interferometer (e.g., spectral-domain OCT (SD-OCT), swept-source OCT (SS-OCT), multimodal OCT (MM-OCT), Intravascular Ultrasound (IVUS), Near-Infrared Autofluorescence (NIRAF), Near-Infrared Spectroscopy (NIRS), Near-Infrared Fluorescence (NIRF), therapy modality using light, sound, or other source of radiation, etc.).

Further, it is a broad object of the present disclosure to provide one or more methods or techniques that operate to one or more of the following: (i) perform optical probe or catheter calibration automatically for, or associated with, an entire or whole pullback of a catheter or probe for one or more intravascular images (such as, but not limited to, OCT images); (ii) reduce computational time to characterize the pullback and/or automatically calibrate a probe or catheter in one or more embodiments; (iii) automatically calibrate the probe or catheter in any environmental material/condition (e.g., blood versus air, in vivo versus ex vivo, or other condition/material change(s)) without having to detect detailed structure(s) (e.g., a sheath, an artifact, a whole catheter sheath, etc.); (iv) automatically calibrate a probe or catheter regardless of noise (e.g., even noisy probes or catheters may be automatically calibrated); (v) apply in vivo and ex vivo calibration feature(s) to the probe or catheter to ensure that the probe or catheter is calibrated for and compatible with any environment; (vi) apply one or more features of the present disclosure to achieve a probe or catheter that may be calibrated in different clinical calibration scenarios (e.g., hand touch, table and air calibration, etc.); (vii) use automatic skeleton sheath detection (e.g., by automatically selecting/detecting a skeleton of a sheath: (1) a detailed sheath detection is not required; (2) any possible artifact detection/removal that would be used or required for a detailed sheath detection is not required/needed (e.g., multiple ring formation); and/or (3) a shape (e.g., an oval shape or any other geometric shape) of the probe or catheter would not or may not affect a final result for the calibration; etc.); (viii) achieve detection of a blood position to automatically locate a position of a sheath, re-adjust an image, and re-calibrate a probe or catheter that may be affected by environmental changes/materials (e.g., in vivo calibration); and/or (ix) perform a more detailed tissue detection or characterization and/or imaging in one or more embodiments. One or more embodiments of the present disclosure overcome the aforementioned issue(s) of having probe(s) or catheter(s) be affected by change(s) in environmental material(s)/condition(s), by stretching, and/or by or from artifacts (e.g., where a probe or catheter may have sensitivities being affected by artifacts). Indeed, several methodologies of the present disclosure have been developed which use an apparatus, a system, a method, a storage medium, etc. that operate to achieve or do one or more of the aforementioned items: (i) through (ix).

As aforementioned, the fiber optic catheters and endoscopes of the present disclosure have been developed to access internal organs, tissues, or other targets, samples, or objects. For example, OCT (optical coherence tomography), white light back-reflection, NIRS (near infrared spectroscopy), and fluorescence technology have been developed to see structural and/or molecular images of vessels with a catheter. The catheter, which comprises a sheath and an optical probe in one or more embodiments, may be navigated to a target, sample, or object, such as, but not limited to, a coronary artery in a cardiology application(s).

In order to acquire cross-sectional images of tubes and cavities, such as, but not limited to, vessels, an esophagus, and at least one nasal cavity, the optical probe may be rotated with a fiber optic rotary joint (FORJ). In addition, the optical probe may be simultaneously translated longitudinally during the rotation so that helical scanning pattern images are obtained. This translation may be performed by pulling the tip of the probe back towards a proximal end, and this translation is, therefore, referred to as a pullback. While particular tubes, cavities, or other targets, samples, or objects (e.g., coronary arteries) may be discussed herein, the tubes/cavities, targets, samples, or objects for which the features of the present disclosure may be used are not limited thereto. Additionally, while particular imaging modalities that may be used in combination are discussed herein (e.g., an intravascular OCT and fluorescence system), the imaging modalities that may be used with one or more features of the present disclosure are not limited thereto.

In one or more embodiments, an optical system may include: an interference optical system that operates to: (i) receive and divide light from a light source into a first light with which an object or sample is to be irradiated and which travels along a sample arm of the interference optical system and a second reference light, (ii) send the second reference light along a reference arm of the interference optical system for reflection off of a reference reflection of the interference optical system, and (iii) generate interference light by causing reflected or scattered light of the first light with which the object or sample has been irradiated and the reflected second reference light to combine or recombine, and to interfere, with each other, the interference light generating one or more interference patterns; one or more detectors that operate to continuously acquire the interference light and/or the one or more interference patterns to measure the interference or the one or more interference patterns between the combined or recombined light to obtain data for one or more imaging modalities, wherein a wavelength of the first light is shorter than a wavelength of the reflected or scattered light and/or the generated interference light. In one or more embodiments, the interference optical system or a probe of the interference optical system may include a double clad fiber.

In one or more embodiments, the one or more imaging modalities may include one or more of the following: Optical Coherence Tomography (OCT), single modality OCT, multi-modality OCT, swept source OCT, optical frequency domain imaging (OFDI), intravascular ultrasound (IVUS), another lumen image(s) modality, near-infrared spectroscopy (NIRS), near-infrared fluorescence (NIRF), near-infrared auto-fluorescence (NIRAF), near-infrared, fluorescence, and an intravascular imaging modality.

In one or more embodiments, an imaging apparatus may include: an interference optical system that operates to: (i) receive and divide light from a light source into a first light with which an object or sample is to be irradiated and which travels along a sample arm of the interference optical system and a second reference light, (ii) send the second reference light along a reference arm of the interference optical system for reflection off of a reference reflection of the interference optical system, and (iii) generate interference light by causing reflected or scattered light of the first light with which the object or sample has been irradiated and the reflected second reference light to combine or recombine, and to interfere, with each other, the interference light generating one or more interference patterns; and one or more detectors that operate to continuously acquire the interference light and/or the one or more interference patterns to measure the interference or the one or more interference patterns between the combined or recombined light to obtain data for one or more imaging modalities, wherein a wavelength of the first light is shorter than a wavelength of the reflected or scattered light and/or the generated interference light. In one or more embodiments, one or more of the following may occur: (i) the one or more detectors operate to continuously acquire the interference light and/or the one or more interference patterns in the interference optical system, optical probe, or catheter.

An imaging apparatus may include one or more processors that operate to perform a pullback of the optical probe or the catheter and/or obtain one or more images or frames of one or more imaging modalities from the pullback of the optical probe or the catheter. In one or more embodiments, the one or more imaging modalities may include one or more of the following: Optical Coherence Tomography (OCT), single modality OCT, multi-modality OCT, swept source OCT, optical frequency domain imaging (OFDI), intravascular ultrasound (IVUS), another lumen image(s) modality, near-infrared spectroscopy (NIRS), near-infrared fluorescence (NIRF), near-infrared auto-fluorescence (NIRAF), near-infrared, fluorescence, and/or an intravascular imaging modality. The one or more processors may further operate to display the one or more images on a display, store the one or more images in a memory, or use the one or more images to train one or more models or AI-networks to auto-detect or to perform automatic calibration and/or to automatically obtain one or more images of the one or more imaging modalities. One or more of the following may occur: (i) the trained model may be one or a combination of the following: a neural net model or neural network model, a deep convolutional neural network model, a recurrent neural network model with long short-term memory that can take temporal relationships across images or frames into account, a generative adversarial network (GAN) model, a consistent generative adversarial network (cGAN) model, a three cycle-consistent generative adversarial network (3cGAN) model, a model that can take temporal relationships across images or frames into account, a model that can take temporal relationships into account including tissue location(s) and/or probe or catheter location(s) during pullback in a vessel and/or including tissue and/or calibration characterization data during pullback in a vessel, a model that can use prior knowledge about a procedure and incorporate the prior knowledge into the machine learning algorithm or a loss function, a model using feature pyramid(s) that can take different image resolutions into account, and/or a model using residual learning technique(s), a segmentation model, a segmentation model with post-processing, a model with pre-processing, a model with post-processing, a segmentation model with pre-processing, a deep learning or machine learning model, a semantic segmentation model or classification model, an object detection or regression model, an object detection or regression model with pre-processing or post-processing, a combination of a semantic segmentation model and an object detection or regression model, a model using repeated segmentation model technique(s), a model using feature pyramid(s), a genetic algorithm that operates to breed multiple models for improved performance, and/or a model using repeated object detection or regression model technique(s); and/or (ii) the one or more processors may further operate to use one or more neural networks or convolutional neural networks to one or more of: load a trained model of images including probe or catheter area(s); perform automatic calibration on the optical probe and/or the catheter; determine whether the calibrated probe or catheter is/are accurate or correct; determine one or more of the characteristics of one or more objects, targets, or samples in the one or more images; identify or detect the one or more objects, targets, or samples; overlay data on at least one of the one or more images to show location(s) of intravascular image(s), the calibrated probe or catheter, or the objects, targets, or samples; incorporate image processing and machine learning (ML) or deep learning to automatically perform calibration of the interference optical system, the optical probe, or the catheter; incorporate image processing and machine learning (ML) or deep learning to automatically identify and locate the one or more objects, targets, or samples; display the results for the automatic calibration and/or tissue or probe/catheter characterization on a display; and/or acquire or receive image data during the pullback operation of the catheter or the optical probe.

In a case where the interference optical system, the optical probe or catheter, or one or more components of the optical probe or catheter include or are attached to a double clad fiber, one or more of the following may exist: (i) the imaging apparatus further comprises one or more processors that operate to perform a pullback of the optical probe or the catheter and/or obtain one or more images or frames of one or more imaging modalities from the pullback of the optical probe or the catheter; (ii) the imaging apparatus further comprises: one or more processors that operate to perform a pullback of the optical probe or the catheter and/or obtain one or more images or frames of one or more imaging modalities from the pullback of the optical probe or the catheter, and the one or more processors further operate to automatically calibrate the interference optical system, the optical probe, or the catheter; and/or (iii) the interference optical system may further include an OCT sub-system and a sub-system for another imaging modality.

In one or more embodiments, a method for performing automatic calibration of an interference optical system, an optical probe, and/or one or more components of the optical probe and/or of a catheter of an imaging apparatus may include: performing an ex vivo calibration and an in vivo calibration. In one or more embodiments, a method for ex vivo and in vivo calibrating a catheter or probe of an apparatus may include: obtaining or receiving one or more images or one or more A-line images; automatically calibrating the catheter or probe using an ex vivo calibration, before the catheter or probe is inserted into a target, sample, or object, by detecting one or more skeletons or portions of a sheath of the catheter or probe and determining whether the skeletons or portions of the sheath are in a target or set position; and automatically calibrating the catheter or probe using an in vivo calibration, after the catheter or probe is inserted into the target, sample, or object, by detecting blood or a blood border position to automatically locate a position of the one or more skeletons or portions of the sheath and then adjusting or re-adjusting the image or the A-line image and calibrating or re-calibrating the catheter or probe to reduce or remove any effects caused by in vivo or environmental changes.

In one or more embodiments, a computer-readable storage medium may store at least one program that operates to cause one or more processors to execute a method for ex vivo and in vivo calibrating a catheter or probe of an apparatus. In one or more embodiments, a computer-readable storage medium storing at least one program that operates to cause one or more processors to execute a method for ex vivo and in vivo calibrating a catheter or probe of an apparatus, where the method may include: obtaining or receiving one or more images or one or more A-line images; automatically calibrating the catheter or probe using an ex vivo calibration, before the catheter or probe is inserted into a target, sample, or object, by detecting one or more skeletons or portions of a sheath of the catheter or probe and determining whether the skeletons or portions of the sheath are in a target or set position; and automatically calibrating the catheter or probe using an in vivo calibration, after the catheter or probe is inserted into the target, sample, or object, by detecting blood or a blood border position to automatically locate a position of the one or more skeletons or portions of the sheath and then adjusting or re-adjusting the image or the A-line image and calibrating or re-calibrating the catheter or probe to reduce or remove any effects caused by in vivo or environmental changes.

In one or more embodiments, a computer-readable storage medium storing at least one program that operates to cause one or more processors to execute a method for performing automatic calibration of an interference optical system, an optical probe, and/or one or more components of the optical probe and/or of a catheter of an imaging apparatus may be used where the method may include any feature discussed herein, including, but not limited to: using an excitation laser or light with a wavelength of a predetermined range or value on or in the interference optical system, the optical probe, and/or one or more components of the optical probe and/or of a catheter for a predetermined or set amount of time or more to perform the automatic calibration for the interference optical system, the optical probe, and/or the one or more components of the optical probe and/or of the catheter.

In one or more embodiments, the object, target, or sample may include one or more of the following: a vessel; a target, a specimen, or object; a tissue or tissues; a patient; an interference optical system; one or more optical probes or catheters; and/or one or more components of the one or more optical probes or catheters.

The one or more processors may further operate to perform the coregistration by co-registering an acquired or received angiography image or the constructed image (e.g., a carpet view) and an obtained one or more intravascular images, such as, but not limited to, OCT or IVUS images or frames.

In one or more embodiments, a loaded, trained model may be one or a combination of the following: a segmentation (classification) model, a segmentation model with pre-processing, a segmentation model with post-processing, an object detection (regression) model, an object detection model with pre-processing, an object detection model with post-processing, a combination of a segmentation (classification) model and an object detection (regression) model, a deep convolutional neural network model, a recurrent neural network model with long short-term memory that can take temporal relationships across images or frames into account, a model using feature pyramid(s) that can take different image resolutions into account, a genetic algorithm that operates to breed multiple models for improved performance (as compared with a case where the genetic algorithm is not used), a model using residual learning technique(s), and/or any other model discussed herein or known to those skilled in the art.

In one or more embodiments, the one or more processors may further operate to one or more of the following: (i) display an image for each of one or more imaging modalities on a display, wherein the one or more imaging modalities include one or more of the following: a tomography image; an Optical Coherence Tomography (OCT) image; a fluorescence image; a near-infrared auto-fluorescence (NIRAF) image; a near-infrared auto-fluorescence (NIRAF) image in a predetermined view, a carpet view, and/or an indicator view; a near-infrared fluorescence (NIRF) image, a near-infrared fluorescence (NIRF) image in a predetermined view, a carpet view, and/or an indicator view; a near-infrared spectroscopy (NIRS) image; a three-dimensional (3D) rendering; a 3D rendering of a vessel; a 3D rendering of a vessel in a half-pipe view or display; a 3D rendering of the object; a lumen profile; a lumen diameter display; a longitudinal view; computer tomography (CT); Magnetic Resonance Imaging (MRI); Intravascular Ultrasound (IVUS); an X-ray image or view; and an angiography view; and (ii) change or update the displays based on the tissue(s) or tissue characteristic(s) evaluation results, based on the automatic calibration results, and/or based on an updated location of the probe or catheter.

Turning now to the details of the figures, imaging modalities may be displayed in one or more ways as discussed herein. One or more displays discussed herein may allow a user of the one or more displays to use, control and/or emphasize multiple imaging techniques or modalities, such as, but not limited to, OCT, CT, IVUS, NIRF, NIRAF, fluorescence, NIRS, etc., and may allow the user to use, control, and/or emphasize the multiple imaging techniques or modalities synchronously.

As shown diagrammatically in FIG. 1A, one or more embodiments for visualizing, emphasizing and/or controlling one or more imaging modalities and for performing automatic calibration of an optical interference system and/or a catheter/probe, evaluating and detecting or identifying one or more tissue types and/or tissue characteristics, and/or performing coregistration of the present disclosure may be involved with one or more predetermined or desired procedures, such as, but not limited to, performing automatic calibration of an optical interference system and/or a catheter/probe, medical procedure planning and performance (e.g., Percutaneous Coronary Intervention (PCI)), etc. For example, the system 2 (e.g., a computer system 2) may communicate with the image scanner 5 (e.g., a CT scanner, an X-ray machine, etc.) to request information for use in the medical procedure (e.g., PCI) planning and/or performance, such as, but not limited to, bed positions, and the image scanner 5 may send the requested information along with the images to the system 2 once a clinician uses the image scanner 5 to obtain the information via scans of the patient. In some embodiments, one or more angiograms 3 taken concurrently or from an earlier session are provided for further planning and visualization. The system 2 may further communicate with a workstation such as a Picture Archiving and Communication System (PACS) 4 to send and receive images of a patient to facilitate and aid in the medical procedure planning and/or performance. Once the plan is formed, a clinician may use the system 2 along with a medical procedure/imaging device 1 (e.g., an imaging device, an OCT device, an IVUS device, a PCI device, an ablation device, a 3D structure construction or reconstruction device, etc.) to consult a medical procedure chart or plan to understand the shape and/or size of the targeted biological object to undergo the imaging and/or medical procedure. Each of the medical procedure/imaging device 1, the system 2, the locator device 3, the PACS 4 and the scanning device 5 may communicate in any way known to those skilled in the art, including, but not limited to, directly (via a communication network) or indirectly (via one or more of the other devices such as 1 or 5, or additional flush and/or contrast delivery devices; via one or more of the PACS 4 and the system 2; via clinician interaction; etc.).

In medical procedures, improvement or optimization of physiological assessment is preferable to decide a course of treatment for a particular patient. By way of at least one example, physiological assessment is very useful for deciding treatment for cardiovascular disease patients. In a catheterization lab, for example, physiological assessment may be used as a decision-making tool—e.g., whether a patient should undergo a PCI procedure, whether a PCI procedure is successful, etc. While the concept of using physiological assessment is theoretically sound, physiological assessment still waits for more adoption and/or adaptation and improvement for use in the clinical setting(s). This situation may be because physiological assessment may involve adding another device and medication to be prepared, and/or because a measurement result may vary between physicians due to technical difficulties. Such approaches add complexities and lack consistency. Therefore, one or more embodiments of the present disclosure may employ computational fluid dynamics based (CFD-based) physiological assessment that may be performed from imaging data to eliminate or minimize technical difficulties, complexities and inconsistencies during the measurement procedure. To obtain accurate physiological assessment, an accurate 3D structure of the vessel may be reconstructed from the imaging data as disclosed in U.S. Provisional Pat. App. No. 62/901,472, filed on Sep. 17, 2019, the disclosure of which is incorporated by reference herein in its entirety. Additionally or alternatively, the determination or identification of one or more tissue types and/or tissue characteristics operates to provide additional information for physiological assessment.

In at least one embodiment of the present disclosure, a method may be used to provide more accurate 3D structure(s) compared to using only one imaging modality. In one or more embodiments, a combination of multiple imaging modalities may be used, automatic calibration of an optical interference system and/or a catheter/probe (and/or an image for or taken by same) may be performed, one or more characteristics of calibration may be detected, one or more tissue types and/or tissue characteristics may be detected, and coregistration may be processed/performed using artificial intelligence.

One or more embodiments of the present disclosure may apply machine learning, especially deep learning, to perform automatic calibration of an optical interference system and/or a catheter/probe (and/or an image for or taken by same), detect calibration characteristic(s), detect one or more tissue types and/or tissue characteristics, etc. in an image frame without user input(s) that define an area where intravascular imaging pullback occurs. Using artificial intelligence, for example, deep learning, one or more embodiments of the present disclosure may achieve a better or maximum success rate of performing automatic calibration of an optical interference system and/or a catheter/probe (and/or an image for or taken by same), calibration characteristic(s) detection, tissue type(s) and/or tissue characteristic(s) detection from image data without (or with less) user interactions, and may reduce processing and/or prediction time to display coregistration result(s) based on the improved image quality obtained, including, but not limited to, when using automatic calibration of an optical interference system and/or a catheter/probe (and/or an image for or taken by same), when detecting calibration characteristic(s), and/or when detecting tissue type(s) and/or tissue characteristic(s).

One or more embodiments of the present disclosure may achieve the efficient catheter (or other imaging device) automatic calibration, detection of calibration characteristics(s), detection of tissue type(s) and/or tissue characteristic(s), and/or efficient coregistration result(s) from image(s). In one or more embodiments, the image data may be acquired during intravascular imaging pullback using a catheter (or other imaging device) that may be visualized in an image. In one or more embodiments, a ground truth identifies a location or locations of the catheter or a portion of the catheter (or of another imaging device or a portion of the another imaging device). For example, while not limited hereto, the ground truth may identify a portion of the catheter, an optical probe, and/or one or more portions of an optical interference system. In one or more embodiments, a model has enough resolution to predict the calibration, the tissue type(s), and/or calibration and/or tissue characteristic(s) (e.g., location, size, etc.) in a given image with sufficient accuracy depending on the application or procedure being performed. The performance of the model may be further improved by adding more training data. For example, additional training data may include image annotations, where a user labels or corrects the tissue type(s), tissue characterization(s), and/or catheter detection(s) and/or automatic calibration of an optical interference system and/or a catheter/probe (and/or an image for or taken by same) in each image.

In one or more embodiments, one or more calibration characteristic(s) and/or tissue type(s) or characteristic(s) may be detected and/or monitored using an algorithm, such as, but not limited to, the Viterbi algorithm.

One or more embodiments may automate characterization of tissue(s) and/or identification of tissue type(s) in images using convolutional neural networks (or other AI structure discussed herein or known to those skilled in the art), and may fully automate frame detection on angiographies, intravascular pullbacks, etc. using training (e.g., offline training) and using applications (e.g., online application(s)) to extract and process frames via deep learning.

One or more embodiments of the present disclosure may track and/or calculate automatic calibration, calibration characteristic(s), and/or tissue type(s) and/or tissue characteristic(s) detection or identification success rate(s).

In at least one further embodiment example, a method of 3D reconstruction without adding any imaging requirements or conditions may be employed. One or more methods of the present disclosure may use intravascular imaging, e.g., IVUS, OCT, etc., and one (1) view of angiography. One or more embodiments may use one image only (e.g., carpet view, a frame of carpet views, another frame or image type discussed herein or known to those skilled in the art, etc.) In the description below, while intravascular imaging of the present disclosure is not limited to OCT, OCT is used as a representative of intravascular imaging for describing one or more features herein.

Referring now to FIG. 1B, shown is a schematic diagram of at least one embodiment of an imaging system 20 for performing automatic calibration of an optical interference system and/or a catheter/probe (and/or an image for or taken by same), for detecting automatic calibration or calibration characteristic(s) in a catheter, and/or for generating an imaging catheter path based on a detected location of an imaging catheter, based on tissue type(s) and/or tissue characteristic(s) detection or identification, and/or a regression line representing the imaging catheter path by using an image frame that is simultaneously acquired during intravascular imaging pullback. The embodiment of FIG. 1B may be used with one or more of the artificial intelligence feature(s) discussed herein. The imaging system 20 may include an angiography system 30, an intravascular imaging system 40, an image processor 50, a display or monitor 1209, and an electrocardiography (ECG) device 60. The angiography system 30 may include an X-ray imaging device such as a C-arm 22 that is connected to an angiography system controller 24 and an angiography image processor 26 for acquiring angiography image frames of an object (e.g., any object that may be imaged using the size and shape of the imaging device, a sample, a vessel, a target specimen or object, etc.) or patient 106.

The intravascular imaging system 40 of the imaging system 20 may include a console 32, a catheter 120, and a patient interface unit or PIU 110 that connects between the catheter 120 and the console 32 for acquiring intravascular image frames. The catheter 120 may be inserted into a blood vessel of the patient 106 (or inside a specimen or other target object, inside tissue, etc.). The catheter 120 may function as a light irradiator and a data collection probe that is disposed in a lumen of a particular blood vessel, such as, for example, a coronary artery, or in another type of tissue or specimen. The catheter 120 may include a probe tip, one or more markers or radiopaque markers, an optical fiber, and a torque wire. The probe tip may include one or more data collection systems. The catheter 120 may be threaded in an artery of the patient 106 to obtain images of the coronary artery. The patient interface unit 110 may include a motor M inside to enable pullback of imaging optics during the acquisition of intravascular image frames. The imaging pullback procedure may obtain images of the blood vessel. The imaging pullback path may represent the co-registration path, which may be a region of interest or a targeted region of the vessel.

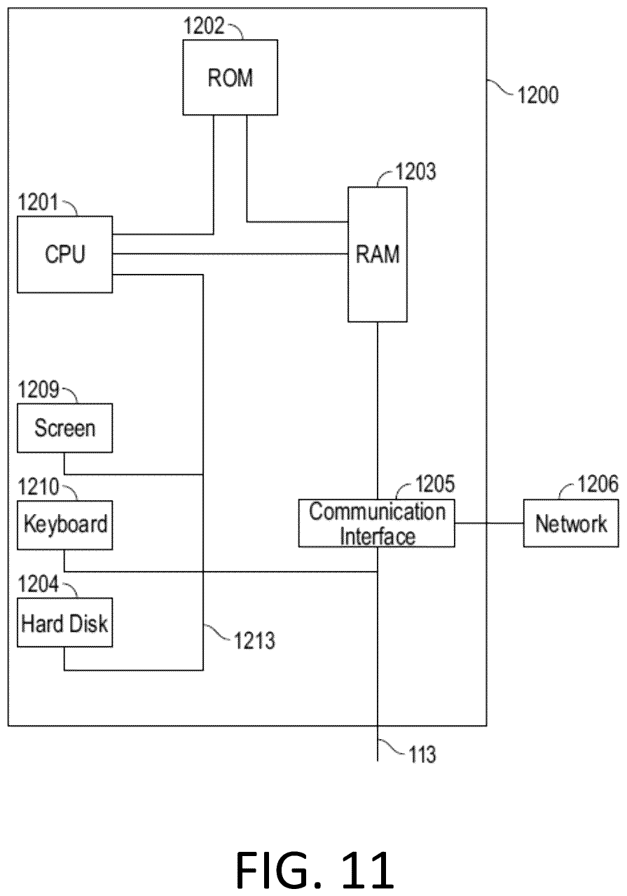

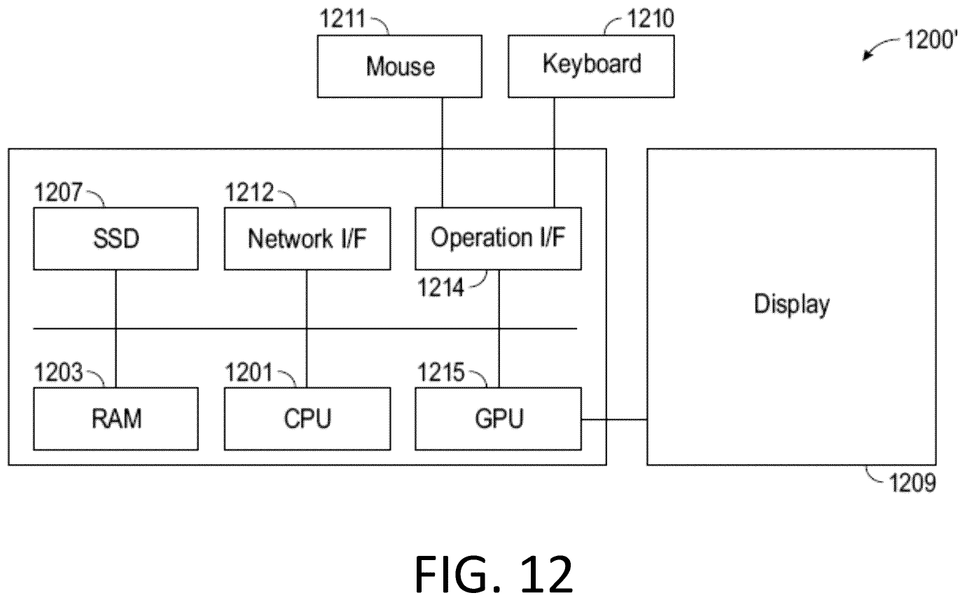

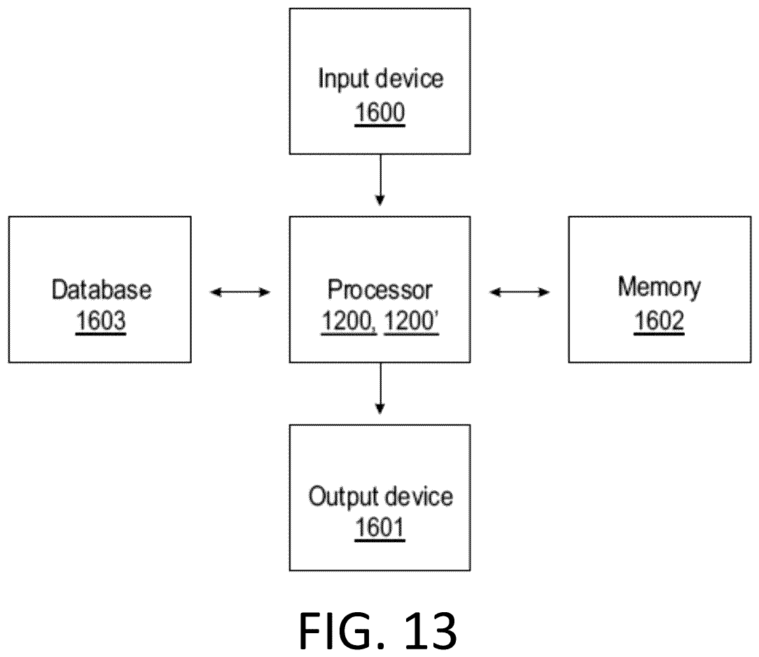

The console 32 may include a light source(s) 101 and a computer 1200. The computer 1200 may include features as discussed herein and below (see e.g., FIG. 11, FIG. 13, etc.), or alternatively may be a computer 1200′ (see e.g., FIG. 12, FIG. 13, etc.) or any other computer or processor, or any combination of computer or processor feature(s), discussed herein. In one or more embodiments, the computer 1200 may include an intravascular system controller 35 and an intravascular image processor 36. The intravascular system controller 35 and/or the intravascular image processor 36 may operate to control the motor M in the patient interface unit 110. The intravascular image processor 36 may also perform various steps for image processing and control the information to be displayed.

Various types of intravascular imaging systems may be used within the imaging system 20. The intravascular imaging system 40 is merely one example of an intravascular imaging system that may be used within the imaging system 20. Various types of intravascular imaging systems may be used, including, but not limited to, an OCT system, a multi-modality OCT system or an IVUS system, by way of example.

The imaging system 20 may also connect to an electrocardiography (ECG) device (or other monitoring device) 60 for recording the electrical activity of the heart (or other organ being monitored, tissue being monitored, specimen being monitored, etc.) over a period of time using electrodes placed on the skin of the patient 106. The imaging system 20 may also include an image processor 50 for receiving angiography data, intravascular imaging data, and data from the ECG device 60 to execute various image-processing steps to transmit to a display 1209 for displaying an angiography image frame with a co-registration path. Although the image processor 50 associated with the imaging system 20 appears external to both the angiography system 20 and the intravascular imaging system 30 in FIG. 1B, the image processor 50 may be included within the angiography system 30, the intravascular imaging system 40, the display 1209, or a stand-alone device. Alternatively, the image processor 50 may not be required if the various image processing steps are executed using one or more of the angiography image processor 26, the intravascular image processor 36 of the imaging system 20, or any other processor discussed herein (e.g., computer 1200, computer 1200′, computer or processor 2, etc.).

To collect data that may be used to train one or more neural nets, one or more features of an OCT device or system (e.g., an MM-OCT device or system, a SS-OCT device or system, etc.) may be used. Collecting a series of OCT images with or without tissue(s) being shown, with one or more tissue types, with one or more tissue characteristics, with one or more calibration characteristics, with automatic calibration of an optical interference system and/or a catheter/probe (and/or an image for or taken by same) being shown, etc. may result in a plurality (e.g., several thousand) of training images. In one or more embodiments, the data may be labeled based on whether calibration (or automatic calibration) characteristic(s) were identified or detected and/or whether a tissue type was identified or detected, a tissue characteristic was identified or detected, a tissue location was identified or detected, a plurality of tissue types are detected, a plurality of tissue characteristics are detected, etc. (as confirmed by a trained operator or user of the device or system). In one or more embodiments, after at least 30,000 OCT images are captured and labeled, the data may be split into a training population and a test population. In one or more embodiments, data collection may be performed in the same environment or in different environments. For example, during data collection, a flashlight (or any light source) may be used to shine the light down a barrel of an imaging device with no catheter imaging core to confirm that a false positive would not occur in a case where a physician pointed the imaging device at external lights (e.g., operating room lights, a computer screen, etc.). After training is complete, the testing data may be fed through the neural net or neural networks, and the accuracy of the model(s) may be evaluated based on the result(s) of the test data.

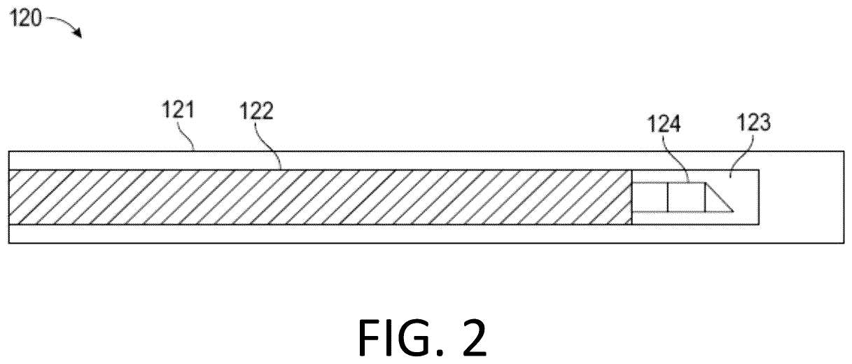

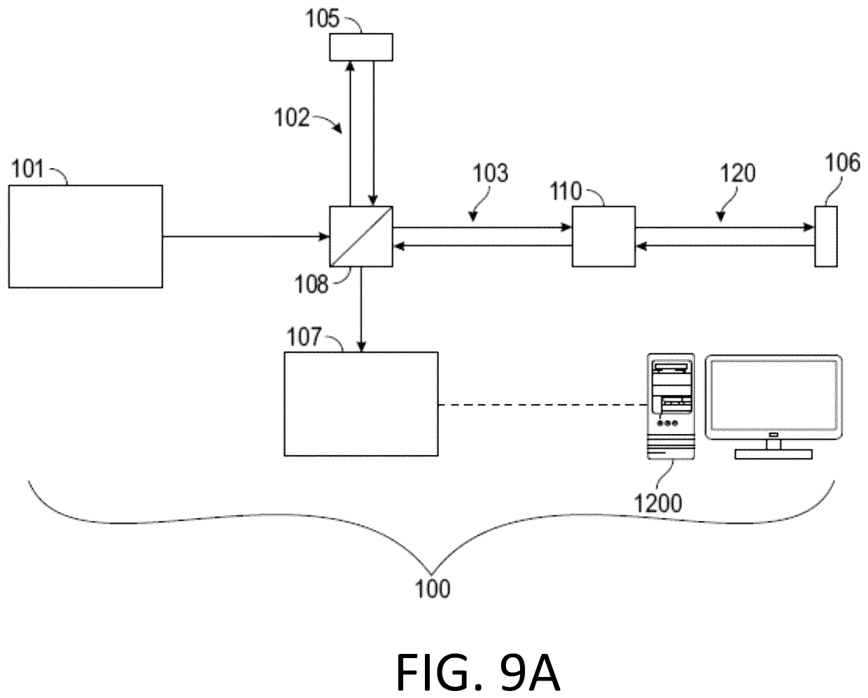

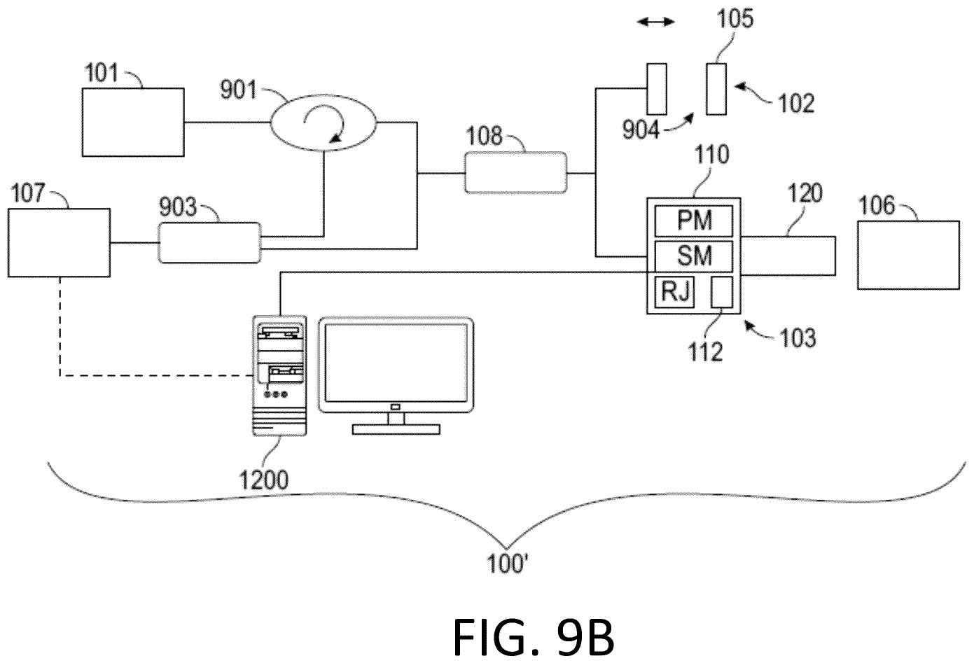

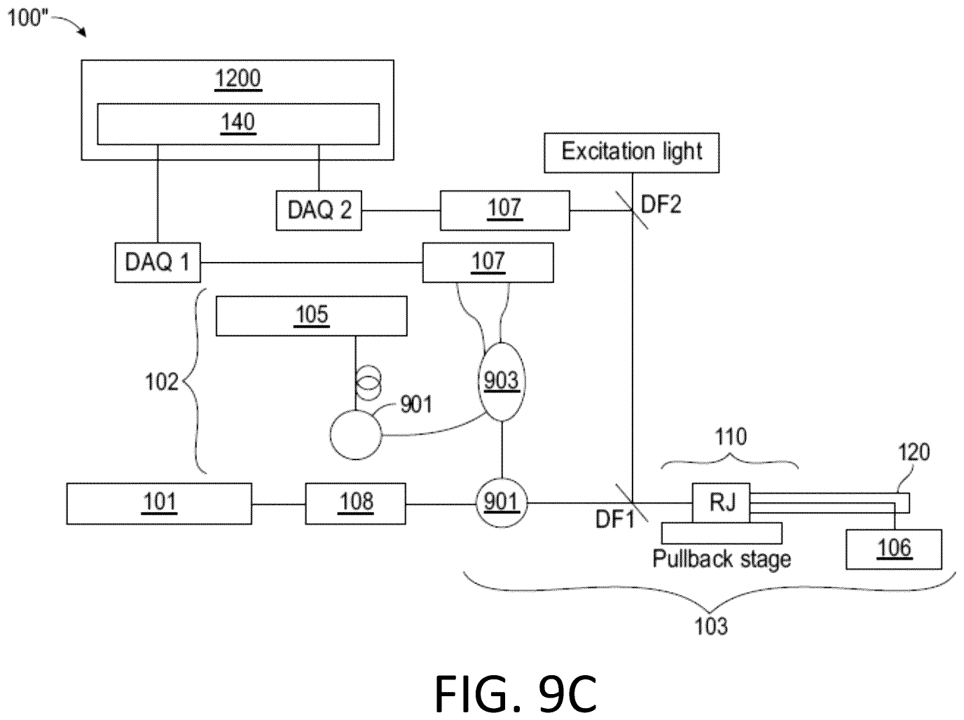

FIG. 2 shows at least one embodiment of a catheter 120 that may be used in one or more embodiments of the present disclosure for obtaining images; for using and/or controlling multiple imaging modalities, to perform automatic calibration of an optical interference system and/or a catheter/probe (and/or an image for or taken by same), to identify or detect calibration characteristic(s), and/or to identify one or more tissue type(s) and/or one or more tissue characteristic(s) in an image or frame with greater or maximum success; and for using the results to perform coregistration more efficiently or with maximum efficiency. FIG. 2 shows an embodiment of the catheter 120 including a sheath 121, a coil 122, a protector 123, and an optical probe 124. As shown schematically in FIGS. 9A-9C (discussed further below), the catheter 120 may be connected to a patient interface unit (PIU) 110 to spin the coil 122 with pullback (e.g., at least one embodiment of the PIU 110 operates to spin the coil 122 with pullback). The coil 122 delivers torque from a proximal end to a distal end thereof (e.g., via or by a rotational motor in the PIU 110). In one or more embodiments, the coil 122 is fixed with/to the optical probe 124 so that a distal tip of the optical probe 124 also spins to see an omnidirectional view of the object (e.g., a biological organ, sample or material being evaluated, such as, but not limited to, hollow organs such as vessels, a heart, a coronary artery, etc.). For example, fiber optic catheters and endoscopes may reside in the sample arm (such as the sample arm 103 as shown in one or more of FIGS. 9A-9C discussed below) of an OCT interferometer in order to provide access to internal organs, such as intravascular images, gastro-intestinal tract or any other narrow area, that are difficult to access. As the beam of light through the optical probe 124 inside of the catheter 120 or endoscope is rotated across the surface of interest, cross-sectional images of one or more objects are obtained. In order to acquire imaging data or three-dimensional data, the optical probe 124 is simultaneously translated longitudinally during the rotational spin resulting in a helical scanning pattern. This translation is most commonly performed by pulling the tip of the probe 124 back towards the proximal end and therefore referred to as a pullback.

The catheter 120, which, in one or more embodiments, comprises the sheath 121, the coil 122, the protector 123 and the optical probe 124 as aforementioned (and as shown in FIG. 2), may be connected to the PIU 110. In one or more embodiments, the optical probe 124, which may be an automatically calibrated optical probe 124 using one or more of the automatic calibration features of the present disclosure, may comprise an optical fiber connector, an optical fiber and a distal lens. The optical fiber connector may be used to engage with the PIU 110. The optical fiber may operate to deliver light to the distal lens. The distal lens may operate to shape the optical beam and to illuminate light to the object (e.g., the object 106 (e.g., a vessel) discussed herein), and to collect light from the sample (e.g., the object 106 (e.g., a vessel) discussed herein) efficiently. While the target, sample, or object 106 may be a vessel in one or more embodiments, the target, sample, or object 106 may be different from a vessel (and not limited thereto) depending on the particular use(s) or application(s) being employed with the catheter 120 (e.g., ex vivo versus in vivo applications). A calibrated optical probe may be fabricated/processed by performing one or more automatic calibration processes to an optical probe, an optical interference system, and/or to a catheter. The optical probe or catheter may emit background emission noise (or catheter background noise) in a case where excitation light couples into an optical fiber of the catheter (e.g., such as the catheter 120). An intensity of the emission noise (or background noise) varies depending on how long an excitation light may go through an optical fiber of the catheter (e.g., the catheter 120).

As aforementioned, in one or more embodiments, the coil 122 delivers torque from a proximal end to a distal end thereof (e.g., via or by a rotational motor in the PIU 110). There may be a mirror at the distal end so that the light beam is deflected outward. In one or more embodiments, the coil 122 is fixed with/to the optical probe 124 so that a distal tip of the optical probe 124 also spins to see an omnidirectional view of an object (e.g., a biological organ, sample or material being evaluated, such as, but not limited to, hollow organs such as vessels, a heart, a coronary artery, etc.). In one or more embodiments, the optical probe 124 may include a fiber connector at a proximal end, a double clad fiber, and a lens at distal end. The fiber connector operates to be connected with the PIU 110. The double clad fiber may operate to transmit & collect OCT light through the core and, in one or more embodiments, to collect Raman and/or fluorescence from an object (e.g., the object 106 (e.g., a vessel) discussed herein, an object and/or a patient (e.g., a vessel in the patient), etc.) through the clad. The lens may be used for focusing and collecting light to and/or from the object (e.g., the object 106 (e.g., a vessel) discussed herein). In one or more embodiments, the scattered light through the clad is relatively higher than that through the core because the size of the core is much smaller than the size of the clad.

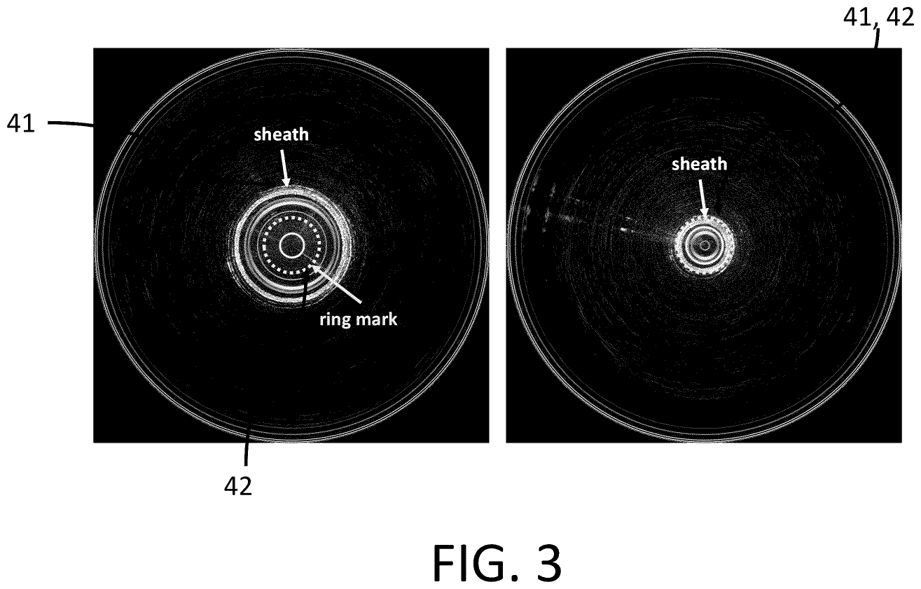

FIG. 3 shows at least one embodiment of an ex vivo path matching process of the present disclosure. The image on the left side of FIG. 3 shows a scenario where a ring mark 42 and the sheath 41 do not match. After a motorized delay line (MDL) is moved in one or more embodiments, the sheath 41 and the ring mark 42 match, and the optical probe or catheter is ex vivo calibrated (as shown in the image on the right side of FIG. 3) and ready to insert the body. In one or more embodiments, images may be input into a model to train the model such that output of the model may result in: (i) data for tracking the MDL to detect, calculate, or identify where the MDL may be moved to achieve image(s) having the sheath 41 and ring mark 42 match or substantially match; and/or (ii) image(s) having the sheath 41 and ring mark 42 match or substantially match as shown on the right side of FIG. 3 (e.g., due to an optimized, identified, determined, or calculated MDL location that operates to achieve such image(s) having the sheath and ring mark match or substantially match).

In one or more embodiments, Optical Coherence Tomography (OCT) may be employed as an interferometric imaging technique. The interferometer may use the optical light traveling from a known optical path (reference path) to interfere with the light returning from an unknown path (sample path). In order for the light interference to correspond to the desired scanned region and any structural measurements to be accurate, one or more embodiments may match the two paths (the reference path and the sample path). Since the optical probes or catheters, which may be a portion of or may define the sample path, may be long sized and since the optical fiber used with or part of the optical probes or catheters may stretch during its use or when changing environmental material(s) or condition(s), one or more embodiments may operate to perform a matching with the reference path.

The matching of the two paths may be performed manually ex vivo (before a probe or catheter is inserted into body), by visually inspecting the position of the sheath of the probe or catheter, the position of the sheath being noticeable in an intracoronary OCT image (see e.g., as shown in FIG. 3). A ring mark (e.g., the ring mark 42 as shown in FIG. 3) in, over, or overlayed on, the OCT cross sectional image may represent the matched sheath area which represents the area that the sheath (e.g., the sheath 41 as shown in FIG. 3) should lie in order for the two paths to be matched or substantially matched. A user may be able (e.g., through software, using one or more processors, via manual interaction(s) with one or more image(s) displayed on a screen/display, etc.) to change the size of the reference path by moving the motorized delay line (MDL). Once the MDL is moved to the determined, identified, calculated, and/or desired length, the two paths may be matched or substantially matched, and the sheath 41 borders are over the ring mark 42. In this way, the zero point for any structural measurements is an outer surface of the sheath of the probe or catheter, and all the distances are measured outward from this location. The manual path matching process is visually shown in FIG. 3.

Such features of one or more path matching processes may be referred to as an OCT catheter calibration and may be performed manually in one or more embodiments of the present disclosure. However, in one or more cases, manual calibration may be time consuming and may lead to inadequate sheath-ring mark matching due to one or more factors, such as, but not limited to, multiple rings appearing inside the sheath, environmental artifacts that appear in the OCT image, etc. The process may be even more difficult when inexperienced users perform it. Moreover, even with a good path matching, the probe or catheter may be frequently re-calibrated in a case where the probe or catheter is inserted into the body/target/sample/object/etc., since the probe or catheter (or a fiber or fibers thereof) may stretch during its use or change due to environmental material(s) or condition(s) (e.g., blood vs. air, any other change discussed herein, etc.). Therefore, the present disclosure provides one or more algorithms that operate to automatically calibrate the probe or catheter in any environmental material (ex vivo and in vivo).

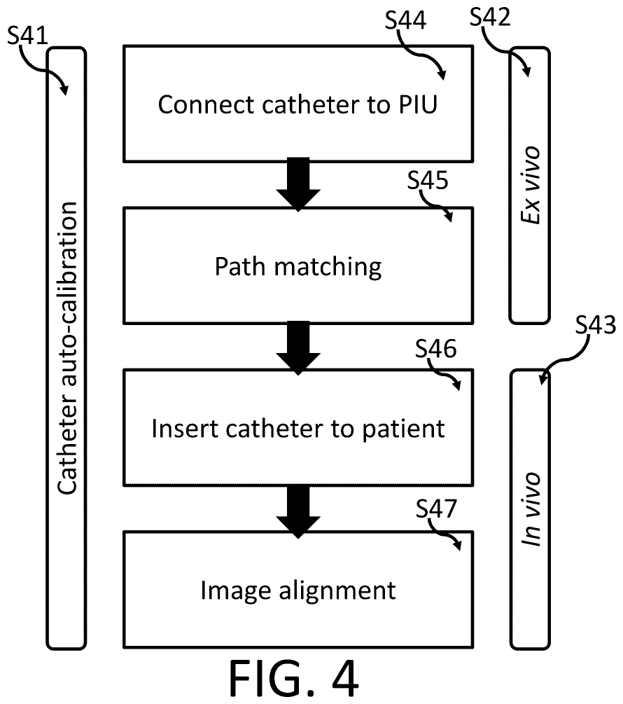

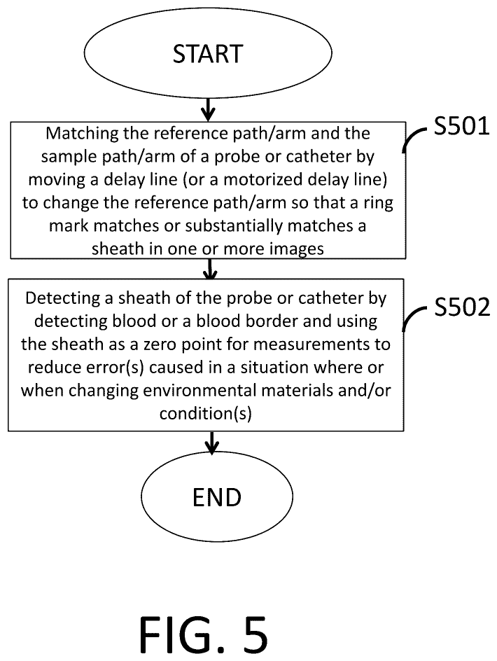

One or more embodiments of a probe or catheter calibration algorithm may include, or be separated into, two parts: (a) path matching, which may happen ex vivo (e.g., before the probe or catheter is inserted into the body/target/sample/object/etc.), and (b) image alignment, which may be performed in-vivo (after the probe or catheter is inserted into the body/target/sample/object/etc.). At least one embodiment of a calibration or automatic calibration algorithm or process step S41 is schematically presented in FIG. 4. Briefly, the ex vivo calibration part step(s) S42 may include or involve (i) connecting the probe or catheter to a patient interface unit (PIU) (see e.g., the PIU 110 as shown in at least FIGS. 1B and 9A-9C and discussed herein) in step S44 of FIG. 4 and/or (ii) performing the matching step S45 of FIG. 4 to match the reference path and the sample path by moving the MDL (e.g., as shown in the right image of FIG. 3 and discussed above), and the in vivo calibration part step(s) S43 may refer to or include (i) inserting the probe or catheter into the body/target/sample/object/etc. (see e.g., step S46 in FIG. 4) and/or performing the alignment or realignment (see e.g., step S47 in FIG. 4) of the OCT image by detecting the sheath (e.g., via detection of blood or a blood border) and using the sheath as the zero point for measurements in order to reduce the errors caused in a situation where or when changing the environmental material(s) and/or condition(s). As shown in FIG. 5, one or more embodiments of the present disclosure may not be limited to ex vivo or in vivo, and may be performed by: (i) matching the reference path/arm and the sample path/arm of a probe or catheter by moving a delay line (or a motorized delay line) to change the reference path/arm so that a ring mark matches or substantially matches a sheath in one or more images (e.g., as shown in step S501 of FIG. 5); and (ii) identifying, marking, detecting, or otherwise determining a sheath of the probe or catheter and using the sheath as a zero point for measurements to reduce error(s) caused in a situation where or when changing environmental materials and/or condition(s) (e.g., as shown in step S502 in FIG. 5). While one or more embodiments may detect a sheath of a probe or catheter via detection of blood or a blood border, such embodiments of the present disclosure are not limited thereto. For example, in one or more embodiments, a sheath of a probe or catheter may be detected, identified, marked, or determined by a user or may be automatically detected, identified, marked, or determined by one or more processors based on set or predetermined data or characteristics for the sheath. By way of another example, in one or more embodiments (and while not limited hereto), one or more images of a sheath may be input into a model to train the model to identify the sheath such that the output of the model may include the one or more images having the sheath in each of the one or more images be identified.

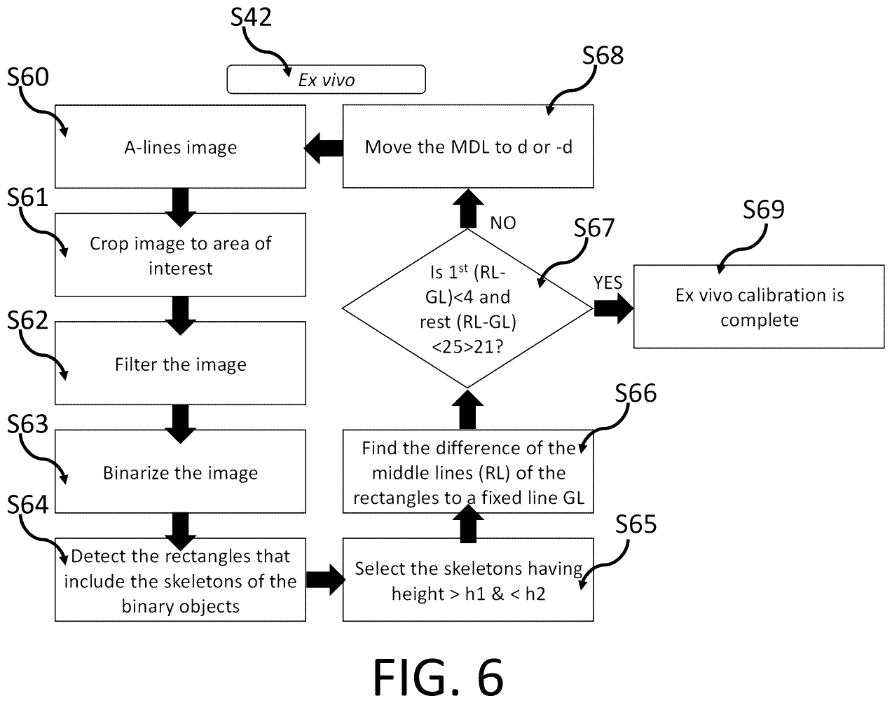

In accordance with one or more aspects of the present disclosure, one or more methods for performing image calibration or automatic calibration are provided herein. In one or more embodiments of calibration processes/methods using one or more ex vivo calibration features, the ex vivo calibration step S42 (e.g., after the probe or catheter is connected to the PIU 110 the ex vivo calibration step S42 may be performed, the ex vivo calibration step S42 may be performed before the probe or catheter is inserted into the target/sample/body/object/etc.; etc.) may be performed using one or more of the following steps (as schematically illustrated in FIG. 6): (i) acquiring an A-line image (e.g., an OCT image in polar coordinates) (see step S60 in FIG. 6) (for example, in one or more embodiments, the calibration method(s) may apply the ex vivo algorithm S42 steps (step S44 and/or step S45 of FIG. 4) to the A-line image (e.g., an OCT image in polar coordinates) to check whether the probe or catheter is calibrated or not); (ii) cropping an image to an area of interest (see step S61 in FIG. 6); (iii) filtering the image (see step S62 in FIG. 6); (iv) binarizing the image (see step S63 in FIG. 6); (v) detecting the rectangles that include the skeletons of the binary objects (see step S64 in FIG. 6); (vi) selecting the skeletons having height >h1 and <h2 (e.g., where h1 and h2 may be set by a user; where h1 and h2 may be calculated or predetermined by one or more processors; etc.) (see step S65 in FIG. 6); (vii) finding a difference of the middle lines (RL) of the rectangles (or other shapes) to a fixed line (GL) (see step S66 in FIG. 6); (viii) determining whether the 1st (RL-GL) (which may represent the rectangle of the sheath object)<4 and the rest (RL-GL)<than 21 through 25 or is between 25 and 21 (see step S67 in FIG. 6); and (ix) if “YES” in step S67, then the probe or catheter is ex vivo calibrated and the calibration is completed (see step S69 in FIG. 6); or, if “NO” in step S67, then (x) the MDL is moved to d or −d, where d or −d is a step to be defined (see step S68 in FIG. 6), and the steps of S60 through S67 are repeated such that a new A-line image is acquired in step S60 and steps S61 through S67 are repeated for the new A-line image. In one or more embodiments, d or −d may be a distance step in a set or predetermined increment of MDL movement. For example, and while not limited hereto, one or more embodiments may use millimeter (mm) increments (e.g., 1 mm increments, 2 mm increments, one or more mm increments, a set or predetermined mm increment, etc.). One or more embodiments may allow a processor and/or user to set the predetermined increment.

### Ex Vivo Calibration:

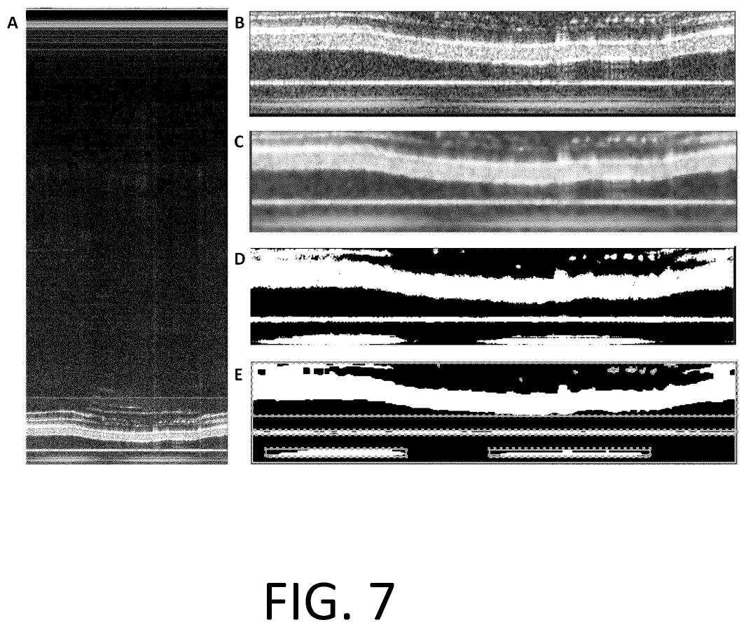

Once a catheter or probe is connected, the ex vivo calibration part may begin or may start. The first step S60 of FIG. 6 may include the acquisition of one A-line image, for example, as shown in FIG. 7A (one or more ex vivo steps or features applied in or to a calibrated image may be performed as illustrated in FIGS. 7A-7E). In step S61, the image may be cropped to an area of interest (for example, as shown in FIG. 7B) which may correspond to the ring area 42 shown in FIG. 3. The cropped image may be filtered using at least one bilateral filtering method where the resulting filtered image may be, for example, as shown in FIG. 7C.

### Bilateral Filtering:

Similarly to Gaussian filters, bilateral filters are non-linear smoothing filters. The fundamental difference is that bilateral filters take into account the pixels intensity differences, which result in achieving edge maintenance simultaneously with noise reduction. Using convolutions, a weighted average of the neighborhood pixels' intensities may replace the intensity of the mask's central pixel. In one or more embodiments, the bilateral filter for an image I, and a window mask W is defined as:

$${I}^{′} ( x ) = \frac{1}{{W}_{p}} {\sum }_{{x}_{i} ∈ W} I ( {x}_{i} ) {f}_{r} ( ‖ I ( {x}_{i} ) - I ( x ) ‖ ) {g}_{s} ( ‖ {x}_{i} - x ‖ ) ,$$

having a normalization factor Wₚ: Wₚ=Σₓ_{ᵢ}_{∈w} fᵣ(∥I(xᵢ)−I(x)∥)gₛ(∥xᵢ−x∥), where x are the coordinates of the mask's central pixel and the parameters fᵣ and gₛ are the Gaussian kernel for smoothing differences in intensities and the spatial Gaussian kernel for smoothing differences in coordinates.

Then, in one or more embodiments, the image may be automatically thresholded using one or more features of Otsu's thresholding method(s), and several binary objects may be revealed, for example, as shown in FIG. 7D.

### Otsu's Thresholding:

To automatically threshold the carpet view image in one or more embodiments, a threshold Thrₒₜₛᵤ for the image I′ may be calculated using the Otsu's method, and the pixels of the image I′ that are smaller than Thrₒₜₛᵤ may be set to a zero value. The result is a binary image with the guide wire represented by the zero objects.

Since the non-zero objects also may correspond to image artifacts, an extra step may be applied in one or more embodiments: detecting the objects that are smaller than a predetermined area, such as, but not limited to, a whole catheter or probe area, 3% of the whole image, etc. Using this extra step, we ensure that only the objects that correspond to the wall area will be used to detect the border.

In all the detected binary objects, for one or more embodiments, the rectangles or geometric shapes that include each object may be calculated (see e.g., dashed boxes in FIG. 7E). For the rectangles having a height between 3 (h1) and 100 (h2) pixels, calculate a middle line (height/2, RL) for each rectangle, geometric shape, or box and find the absolute difference of a respective middle line of each box to a fixed line GL. The absolute difference may be a difference found between the middle lines (RL) of the rectangles, geometric shapes, or boxes to a fixed line (GL) (see step S66 in FIG. 6). In one or more embodiments, the rectangles, geometric shapes, or boxes may be selected by selecting the skeletons having height >h1 and <h2 (e.g., where h1 and h2 may be set by a user; where h1 and h2 may be calculated or predetermined by one or more processors; etc.) (see step S65 in FIG. 6). GL (Golden line) represents the line of the rectangle binary sheath in a calibrated catheter or probe. If the 1^{st} RL−GL (which should represent the rectangle of the sheath object) is less than 4 and the rest (RL−GL)<than 21 through 25 or is between 25 and 21 (see e.g., step S67 of FIG. 6), then the catheter or probe is ex vivo calibrated; if not, then the MDL is moved to d or −d, where d or −d is a step to be defined (see step S68 in FIG. 6), a new A-line image is acquired, and the process starts again (see e.g., step S68 in FIG. 6 returning to step S60 in FIG. 6 as discussed above).

### In Vivo Calibration:

Once the image or the catheter or probe is ex vivo calibrated, the catheter or probe may be inserted into the body. From this point and on, in one or more embodiments, the MDL may not move, and any calibration error may be corrected by adjusting the image. One or more embodiments of a method or methods of the present disclosure may include one or more of the following: (1) Acquire one A-line image; (2) Apply bilateral filtering and Otsu's thresholding method to the image; (3) Detect the bottom line area of the biggest detected object (The bottom line corresponds to an outer sheath boundary in one or more embodiments); and/or (4) Shift the image such that the detected outer sheath boundary matches or substantially matches the zero point (which may correspond to the ring mark 42 of FIG. 3). By applying the above process, one or more embodiments may achieve the benefit(s) that the zero point for any structural measurements is the outer surface of the sheath of the catheter or probe and that all the distances are measured outward from this location.

### Testing

In accordance with one or more features of the present disclosure, experiments were conducted for the ex vivo calibration part only since the in vivo data collection requires a different environmental setting. Using an MM-OCT catheter or probe, seventeen (17) stationary pullbacks were performed using a non-calibrated catheter or probe, and the MDL was moved by d or −d until an acceptable visual ex vivo calibration was achieved. One image from each pullback was used as input to the ex vivo calibration part of the algorithm or process(es) in order to check whether the catheter or probe is calibrated or not. Details of the experiments, including the result(s) (calibration detected or not) of the performed pullback experiments, is presented in Table 1 (Performed pullbacks and the ex vivo algorithm calibration results):

| Pullback: | | 1 | 2 | 3 | 4 | 5 | 6 | 7 | 8 | 9 | 10 | 11 | 12 | 13 | 14 | 15 | 16 | 17 |
|---|---|---|---|---|---|---|---|---|---|---|---|---|---|---|---|---|---|---|
| MDL | | | | | −d | | | −d | | | −d | | | −d | | | | |
| Environment | Table | + | | | + | | | + | | | + | | | + | | | | |
| | Fingers | | + | | | + | | | + | | | + | | | | | | |
| | Air | | | + | | | + | | | + | | | + | | | + | | |
| | Hand | | | | | | | | | | | | | | + | | + | |
| | palm | | | | | | | | | | | | | | | | | |
| | Table | | | | | | | | | | | | | | | | | + |
| | and | | | | | | | | | | | | | | | | | |
| | Finger | | | | | | | | | | | | | | | | | |

| | | | | | | | | | | | | | | | | | |
|---|---|---|---|---|---|---|---|---|---|---|---|---|---|---|---|---|---|
| Calibrated: | No | No | No | No | No | No | No | No | No | Yes | Yes | Yes | Yes | Yes | Yes | Yes | Yes |
| Ex vivo | No | No | No | No | No | No | No | No | No | No | No | Yes | No | Yes | Yes | Yes | Yes |
| calibration | | | | | | | | | | | | | | | | | |
| detected | | | | | | | | | | | | | | | | | |

In one or more embodiments, a few examples of criteria used to determine whether a visual ex vivo calibration is acceptable may include one or more of the following: whether the sheath of the catheter or probe falls over a predefined or set (e.g., by software, one or more catheter or probe specifications, by a user, via one or more processors, using a threshold, etc.) line or circular line shown in a screen, on a display, in the software, etc.

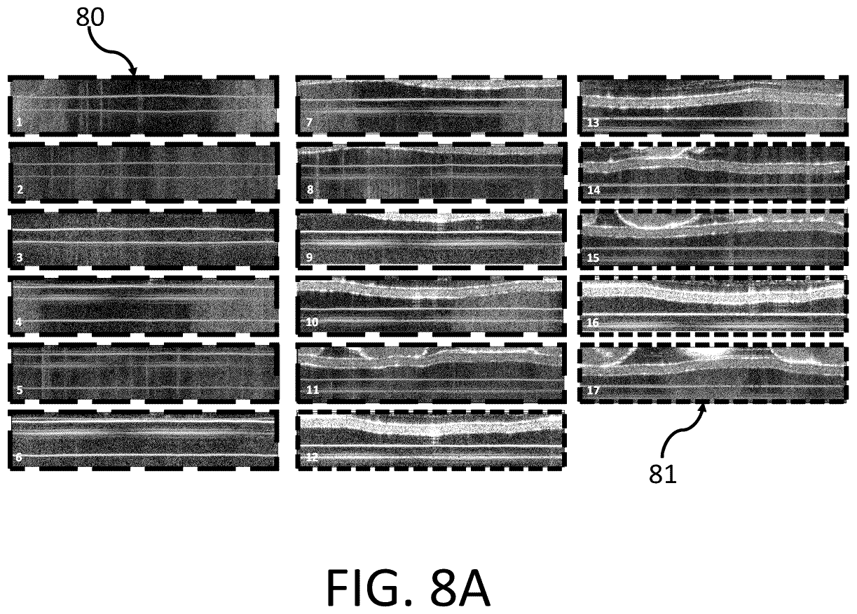

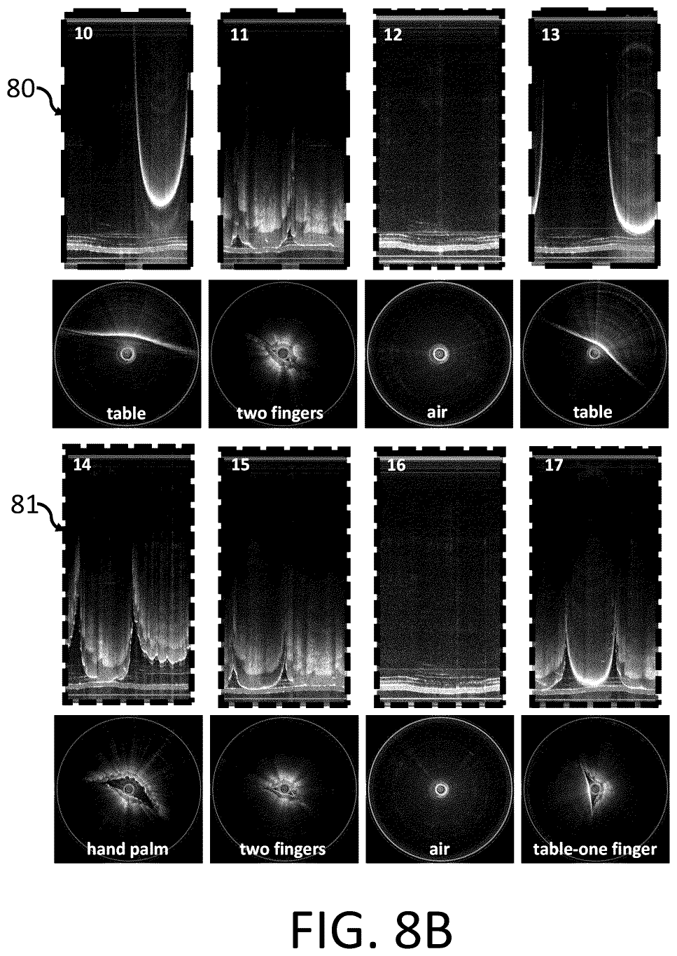

FIG. 8A presents schematically the area of interest for each 1st frame of the seventeen (17) stationary pullbacks having results shown in Table 1. A long dotted or dashed frame (see e.g., the long dotted or dashed frame or box 80 in FIG. 8A) represents that ex vivo calibration was not detected (“No”) by the ex vivo calibration algorithm(s) or process(es). The short dotted or dashed frame (see e.g., the short dotted or dashed frame or box 81 in FIG. 8A) represents that ex vivo calibration was detected (“Yes”) by the ex vivo calibration algorithm(s) or process(es). Pullbacks 10-17 were all considered calibrated pullbacks. Although pullbacks 14-16 differ to d or −d, the subject pullbacks might appear identical in air (see e.g., pullbacks 12 and 16; pullbacks 12 and 15; etc.). The A-line and cross-sectional images for each 1^{st} frame of the calibrated stationary pullbacks (e.g., pullbacks 10-17) are presented in FIG. 8B. As for FIG. 8A, a long dotted or dashed frame (see e.g., the long dotted or dashed frame or box 80 in FIG. 8B) represents that ex vivo calibration was not detected (“No”) by the ex vivo calibration algorithm(s) or process(es). The short dotted or dashed frame (see e.g., the short dotted or dashed frame or box 81 in FIG. 8B) represents that ex vivo calibration was detected (“Yes”) by the ex vivo calibration algorithm(s) or process(es). In one or more embodiments, even in a case where a particular or current pullback occurs and where ex vivo calibration may not be detected (e.g., a false negative may occur), an MDL change to d or −d for a next pullback may be detected as calibrated (e.g., calibration may be confirmed in such a case). In one or more embodiments, d or −d may be such a small value such that the change does not affect the overall or rough calibration happening ex vivo.

One or more embodiments of how to couple OCT and excitation channels into a single core of a double clad fiber in a rotary junction may be used with one or more embodiments of the present disclosure. For example, one or more embodiments of how to couple OCT and excitation channels into a single core of a double clad fiber in a rotary junction may be used as discussed in U.S. Pat. Pub. No. 2018/0348439, published on Dec. 6, 2018, the disclosure of which is incorporated by reference herein in its entirety. One or more features of a rotary joint, a rotary junction, a FORJ, etc. may be used as discussed in U.S. Pat. Pub. No. 2018/0348439, published on Dec. 6, 2018, the disclosure of which is incorporated by reference herein in its entirety.

The one or more calibration (e.g., in vivo calibration, ex vivo calibration, a combination thereof, etc.) method features discussed above may be used for optical probes/components of optical probes (e.g., the optical probe 124, the catheter 120, any component of the catheter 120, etc.). With the calibration process(es)/feature(s) and/or the use of automatic skeleton sheath detection process(es)/feature(s) of the present disclosure, obtaining calibrated optical probes/catheters may be achieved regardless of whether: (i) high noise exists or not; (ii) artifact(s) exist in one or more images or views (e.g., detecting detailed structures, such as, but not limited to, a sheath of the catheter or probe, may be avoided/reduced); and/or (iii) the optical probes/catheters are used in different environments (e.g., hand/palm touch, table, air, table and finger, blood, ex vivo versus in vivo, etc.).

Embodiments of a method or methods for detecting or identifying one or more tissue types and/or tissue characteristics and/or for imaging may be used independently or in combination, including, but not limited to, independently from or in combination with calibration (e.g., ex vivo, in vivo, etc.) features and/or automatic skeleton sheath detection features of the present disclosure.

In one or more embodiments, a model (which, in one or more embodiments, may be software, software/hardware combination, or a procedure that utilizes one or more machine or deep learning algorithms/procedures/processes that has/have been trained on data to make one or more predictions for future, unseen data) has enough resolution to predict and/or evaluate the tissue characterization, the calibration result(s)/estimate(s), and/or the sheath detection estimate(s)/result(s) with sufficient accuracy depending on the application or procedure being performed. The performance of the model may be further improved by subsequently adding more training data and retraining the model to create a new instance of the model with better or optimized performance. For example, additional training data may include data based on user input, where the user may identify or correct the location of a tissue or tissues in an image and/or may identify or correct the sheath location and/or may identify or correct an amount of calibration for the optical probe 124 and/or components of the optical probe 124 and/or of the catheter 120.

One or more methods, medical imaging devices, Intravascular Ultrasound (IVUS) or Optical Coherence Tomography (OCT) devices, imaging systems, and/or computer-readable storage mediums for evaluating tissue characterization(s) and/or for performing calibration and/or sheath detection using artificial intelligence may be employed in one or more embodiments of the present disclosure.

In one or more embodiments, an artificial intelligence training apparatus using a neural network or other AI-ready network may include: a memory; one or more processors in communication with the memory, the one or more processors operating to: training a classifier or patch feature extraction and training an AI-classifier (e.g., a ML classifier, a DL classifier, etc.). In one or more embodiments of the present disclosure, an apparatus, a system, or a storage medium may use an AI network, a neural network, or other AI-ready network to perform any of the aforementioned method step(s), including, but not limited to, the steps of, or related to, FIGS. 4-8B and/or any other step(s) discussed herein.

The one or more processors may further operate to use one or more neural networks, convolutional neural networks, and/or recurrent neural networks (or other AI-ready or AI compatible network(s)) to one or more of: load the trained model, select a set of image frames, evaluate the tissue characterization and/or the calibration and/or sheath detection, construct the image, perform the coregistration and/or the calibration and/or sheath detection, overlay data on the image and/or the intravascular image(s) (e.g., the CVI, the OCT image(s), etc.) and acquire or receive the image data during the pullback operation(s).

In one or more embodiments, the object, target, or sample may include one or more of the following: a vessel, a target specimen or object, one or more tissues, a patient (or a target or tissue(s) in the patient), a sheath or a portion of a sheath, one or more optical probe(s) and/or catheter(s) and/or one or more components of the optical probe(s) and/or catheter(s).

The one or more processors may further operate to perform the coregistration by co-registering an acquired or received angiography image and an obtained one or more intravascular images, such as, but not limited to. Optical Coherence Tomography (OCT) or Intravascular Ultrasound (IVUS) images or frames, and/or by co-registering the carpet view (CVI) with the one or more intravascular images, such as, but not limited to, Optical Coherence Tomography (OCT) or Intravascular Ultrasound (IVUS) images or frames.

In one or more embodiments, a loaded, trained model may be one or a combination of the following: a random forest(s) model, a Support Vector Machine (SVM) model, a segmentation (classification) model, a segmentation model with pre-processing, a segmentation model with post-processing, an object detection (regression) model, an object detection model with pre-processing, an object detection model with post-processing, a combination of a segmentation (classification) model and an object detection (regression) model, a deep convolutional neural network model, a recurrent neural network model with long short-term memory that can take temporal relationships across images or frames into account, a model using feature pyramid(s) that can take different image resolutions into account, a genetic algorithm that operates to breed multiple models for improved performance, and/or a model using residual learning technique(s).

In one or more embodiments, the one or more processors may further operate to one or more of the following: perform any of the steps of (or related to) FIGS. 4-8B, perform any of the steps or feature(s) related to FIGS. 1A-8B, 9A-10, and/or 11-17, and/or any combination of the steps or features discussed in the present disclosure.

One or more embodiments of the present disclosure may use other artificial intelligence technique(s) or method(s) for performing training, for splitting data into different groups (e.g., training group, validation group, test group, etc.), or other artificial intelligence technique(s) or method(s), such as, but not limited to, embodiment(s) as discussed in PCT/US2020/051615, filed on Sep. 18, 2020 and published as WO 2021/055837 A9 on Mar. 25, 2021, and as discussed in U.S. patent application Ser. No. 17/761,561, filed on Mar. 17, 2022, the applications and publications of which are incorporated by reference herein in their entireties. For example, angiography data and/or intravascular data may be used for training, validation, and/or testing as desired. One or more embodiments of a non-transitory computer-readable storage medium storing at least one program for causing a computer to execute a method for training a model using artificial intelligence may be used with any method(s) discussed in the present disclosure, including but not limited to, one or more tissue type and/or characteristic evaluation/determination method(s), calibration method(s), sheath detection method(s), etc.

In accordance with at least one aspect of the present disclosure and as aforementioned, one or more additional methods for target or object detection of OCT images are provided herein and are discussed in U.S. patent application Ser. No. 16/414,222, filed on May 16, 2019 and published on Dec. 12, 2019 as U.S. Pat. Pub. No. 2019/0374109, the entire disclosure of which is incorporated by reference herein in its entirety. By way of a few examples, pre-processing may include, but is not limited to, one or more of the following steps: (1) smoothening a 2D image in a Polar coordinate (e.g., using a Gaussian filter, another type of filter, etc.), (2) computing vertical and/or horizontal gradients using, for example, a Sobel operator, and/or (3) computing binary image using an Otsu's method. For example, Otsu's method is an automatic image thresholding technique to separate pixels into two classes, foreground and background, and the method minimizes the intraclass variances between two classes and is equivalent to a globally optimal k-means (see e.g., https://en.wikipedia.org/wiki/Otsu %27s_method). One skilled in the art would appreciate that pre-processing methods other than Otsu's method (such as, but not limited to, Jenks optimization method) may be used in addition to or alternatively to Otsu's method in one or more embodiments.

By way of at least one embodiment example of a sheath, a Polar coordinate image (e.g., an OCT Polar coordinate image) may include (from the top side to the bottom side, from the top side to the bottom side of an OCT Polar coordinate image, etc.) a sheath area and a normal field of view (FOV). In one or more embodiments, a lumen area and edge may be within the FOV. Because one or more shapes of the sheath may not be a circle (as may be typically assumed) and because the sheath (and, therefore, the sheath shape) may be attached to or overlap with the lumen or guide wire, it may be useful to separate the sheath from the other shapes (e.g., the lumen, the guide wire, tissue(s), etc.) ahead of time.

By way of at least one embodiment example of computing/finding a peak and a major or maximum gradient edge (e.g., for each A-line), soft tissue and other artifacts may be presented on each A-line by one or more peaks with different characteristics, for example, in one or more embodiments of a lumen OCT image(s) (e.g., a normal lumen OCT image). For example, the soft tissue may have a wide bright region beyond the lumen edge, while the artifacts may produce an abrupt dark shadow area beyond the edge. Due to the high-resolution nature of one or more OCT images, transitions between neighbor A-lines may have signals for both peaks. Such signals may allow one or more method embodiments or processes to obtain more accurate locations of the artifact objects and/or the lumen edges. In one or more embodiments, detection of tissue, lumen edges, artifacts, etc. may be performed as discussed in U.S. Pat. Pub. No. 2021/0174125 A1, published on Jun. 10, 2021, the disclosure of which is incorporated by reference herein in its entirety.

Additionally or alternatively, in one or more embodiments, a principal component analysis method and/or a regional covariance descriptor(s) may be used to detect objects, such as tissue(s), and/or to detect, evaluate, and/or perform calibration and/or sheath detection. Cross-correlation among neighboring images may be used to improve tissue characterization and/or detection result(s) and/or to improve calibration estimate(s) and/or result(s). One or more embodiments may employ segmentation based image processing and/or gradient based edge detection to improve result(s).

One or more methods or algorithms for performing co-registration and/or imaging may be used in one or more embodiments of the present disclosure, including, but not limited to, the methods or algorithms discussed in U.S. Pat. App. No. 62/798,885, filed on Jan. 30, 2019, and discussed in U.S. Pat. Pub. No. 2019/0029624, which application(s) and publication(s) are incorporated by reference herein in their entireties.

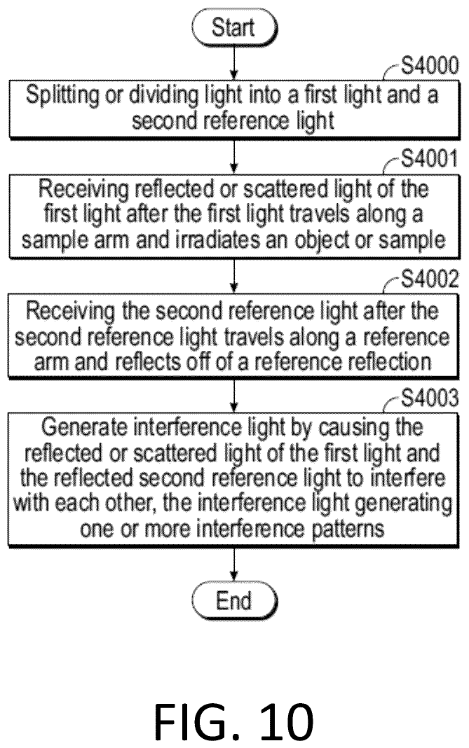

A computer, such as the console or computer 1200, 1200′, may perform any of the steps (e.g., method step(s) related to FIGS. 1A-8B (such as, but not limited to, steps S41-S47 in FIG. 4, steps S501 and S502 in FIG. 5, steps S42-S69 of FIG. 6, etc.); steps S4000-S4003 of FIG. 10 discussed further below; etc.) for any system being manufactured or used, including, but not limited to, system 20, system 100, system 100′, system 100″, any other system discussed herein, etc.

In accordance with one or more further aspects of the present disclosure, bench top systems may be utilized for one or more features of the present disclosure, such as, but not limited to, for one or more imaging modalities (such as, but not limited to, angiography, Optical Coherence Tomography (OCT), Multi-modality OCT (MM-OCT), near-infrared auto-fluorescence (NIRAF), near-infrared fluorescence (NIRF), OCT-NIRAF, OCT-NIRF, etc.), and/or for employing one or more additional features discussed herein, including, but not limited to, artificial intelligence processes (e.g., machine or deep learning, residual learning, artificial intelligence (“AI”) co-registration, tissue detection, tissue characterization, calibration (ex vivo and/or in vivo), sheath detection, etc.) in accordance with one or more aspects of the present disclosure.

FIG. 9A shows an OCT system 100 (as referred to herein as “system 100” or “the system 100”) which may be used for one or more imaging modalities, such as, but not limited to, angiography, Optical Coherence Tomography (OCT), Multi-modality OCT (MM-OCT), near-infrared autofluorescence (NIRAF), near-infrared fluorescence (NIRF), OCT-NIRAF, etc., and/or for employing one or more additional features discussed herein, including, but not limited to, artificial intelligence processes (e.g., machine or deep learning, residual learning, artificial intelligence (“AI”) co-registration, tissue characterization, calibration (ex vivo and/or in vivo), sheath detection, etc.) or other process(es) (e.g., co-registration, tissue detection, tissue characterization, calibration (ex vivo and/or in vivo), sheath detection, etc.) in accordance with one or more aspects of the present disclosure. The system 100 comprises a light source 101, a reference arm 102, a sample arm 103, a deflected or deflecting section 108, a reference mirror (also referred to as a “reference reflection”, “reference reflector”, “partially reflecting mirror” and a “partial reflector”) 105, and one or more detectors 107 (which may be connected to a computer 1200). In one or more embodiments, the system 100 may include a patient interface device or unit (“PIU”) 110 and a catheter or probe 120 (see e.g., embodiment examples of a PIU and a probe or catheter as shown in FIGS. 1A-8B and/or FIGS. 9A-9C), and the system 100 may interact with an object 106, a patient (e.g., a blood vessel of a patient) 106, a sample, one or more tissues, one or more portions or components of an optical probe 124 and/or of the catheter 120, etc. (e.g., via the catheter 120 and/or the PIU 110). In one or more embodiments, the system 100 includes an interferometer or an interferometer is defined by one or more components of the system 100, such as, but not limited to, at least the light source 101, the reference arm 102, the sample arm 103, the deflecting section 108, and the reference mirror 105.

FIG. 9B shows an example of a system that can utilize the one or more imaging modalities, such as, but not limited to, angiography, Optical Coherence Tomography (OCT), Multi-modality OCT (MM-OCT), near-infrared auto-fluorescence (NIRAF), near-infrared fluorescence (NIRF), OCT-NIRAF, OCT-NIRF, etc., and/or can be used for employing one or more additional features discussed herein, including, but not limited to, artificial intelligence processes (e.g., machine or deep learning, residual learning, artificial intelligence (“AI”), or other AI features discussed herein) or other process(es) (e.g., co-registration, tissue detection, tissue characterization, calibration (ex vivo and/or in vivo), sheath detection, etc.) in accordance with one or more aspects of the present disclosure discussed herein for a bench-top such as for ophthalmic applications. A light from a light source 101 delivers and splits into a reference arm 102 and a sample arm 103 with a deflecting section 108. A reference beam goes through a length adjustment section 904 and is reflected from a reference mirror (such as or similar to the reference mirror or reference reflection 105 shown in FIG. 9A) in the reference arm 102 while a sample beam is reflected or scattered from an object, a patient (e.g., blood vessel of a patient), etc. 106 in the sample arm 103 (e.g., via the PIU 110 and the catheter 120). In one embodiment, both beams combine at the deflecting section 108 and generate interference patterns. In one or more embodiments, the beams go to the combiner 903, and the combiner 903 combines both beams via the circulator 901 and the deflecting section 108, and the combined beams are delivered to one or more detectors (such as the one or more detectors 107). The output of the interferometer is continuously acquired with one or more detectors, such as the one or more detectors 107. The electrical analog signals are converted to the digital signals to analyze them with a computer, such as, but not limited to, the computer 1200 (see FIGS. 1B and 9A-9C; also shown in FIGS. 11 and 13 discussed further below), the computer 1200′ (see e.g., FIGS. 12 and 13 discussed further below), the computer 2 (see FIG. 1A), the processors 26, 36, 50 (see FIG. 1B), any other computer or processor discussed herein, etc. Additionally or alternatively, one or more of the computers, CPUs, processors, etc. discussed herein may be used to process, control, update, emphasize, and/or change one or more of imaging modalities, and/or process the related techniques, functions or methods, or may process the electrical signals as discussed above.

The electrical analog signals may be converted to the digital signals to analyze them with a computer, such as, but not limited to, the computer 1200 (see FIGS. 1B and 9A-9C; also shown in FIGS. 11 and 13 discussed further below), the computer 1200′ (see e.g., FIGS. 12 and 13 discussed further below), the computer 2 (see FIG. 1A), any other processor or computer discussed herein, etc. Additionally or alternatively, one or more of the computers, CPUs, processors, etc. discussed herein may be used to process, control, update, emphasize, and/or change one or more imaging modalities, and/or process the related techniques, functions or methods, or may process the electrical signals as discussed above. In one or more embodiments (see e.g., FIG. 9B), the sample arm 103 includes the PIU 110 and the catheter 120 so that the sample beam is reflected or scattered from the object, patient (e.g., blood vessel of a patient), etc. 106 as discussed herein. In one or more embodiments, the PIU 110 may include one or more motors to control the pullback operation of the catheter 120 (or one or more components thereof) and/or to control the rotation or spin of the catheter 120 (or one or more components thereof) (see e.g., the motor M of FIG. 1B). For example, as best seen in FIG. 9B, the PIU 110 may include a pullback motor (PM) and a spin motor (SM), and/or may include a motion control unit 112 that operates to perform the pullback and/or rotation features using the pullback motor PM and/or the spin motor SM. As discussed herein, the PIU 110 may include a rotary junction (e.g., rotary junction RJ as shown in FIGS. 9B and 9C). The rotary junction RJ may be connected to the spin motor SM so that the catheter 120 may obtain one or more views or images of the object, patient (e.g., blood vessel of a patient, tissue(s), etc.), portion(s) or component(s) of the optical probe 124 and/or of the catheter 120, etc. 106. The computer 1200 (or the computer 1200′, computer 2, any other computer or processor discussed herein, etc.) may be used to control one or more of the pullback motor PM, the spin motor SM and/or the motion control unit 112. An OCT system may include one or more of a computer (e.g., the computer 1200, the computer 1200′, computer 2, any other computer or processor discussed herein, etc.), the PIU 110, the catheter 120, a monitor (such as the display 1209), etc. One or more embodiments of an OCT system may interact with one or more external systems, such as, but not limited to, an angio system, external displays, one or more hospital networks, external storage media, a power supply, a bedside controller (e.g., which may be connected to the OCT system using Bluetooth technology or other methods known for wireless communication), etc.

In one or more embodiments including the deflecting or deflected section 108 (best seen in FIGS. 9A-9C), the deflected section 108 may operate to deflect the light from the light source 101 to the reference arm 102 and/or the sample arm 103, and then send light received from the reference arm 102 and/or the sample arm 103 towards the at least one detector 107 (e.g., a spectrometer, one or more components of the spectrometer, another type of detector, etc.). In one or more embodiments, the deflected section (e.g., the deflected section 108 of the system 100, 100′, 100″, any other system discussed herein, etc.) may include or may comprise one or more interferometers or optical interference systems that operate as described herein, including, but not limited to, a circulator, a beam splitter, an isolator, a coupler (e.g., fusion fiber coupler), a partially severed mirror with holes therein, a partially severed mirror with a tap, etc. In one or more embodiments, the interferometer or the optical interference system may include one or more components of the system 100 (or any other system discussed herein) such as, but not limited to, one or more of the light source 101, the deflected section 108, the rotary junction RJ, a PIU 110, a catheter 120, etc. One or more features of the aforementioned configurations of at least FIGS. 1A-9B (and/or any other configurations discussed below) may be incorporated into one or more of the systems, including, but not limited to, the system 20, 100, 100′, 100″, etc. discussed herein.

In accordance with one or more further aspects of the present disclosure, one or more other systems may be utilized with one or more of the multiple imaging modalities and related method(s) as disclosed herein. FIG. 9C shows an example of a system 100″ that may utilize the one or more multiple imaging modalities, such as, but not limited to, angiography, Optical Coherence Tomography (OCT), Multi-modality OCT (MM-OCT), near-infrared auto-fluorescence (NIRAF), near-infrared fluorescence (NIRF), OCT-NIRAF, OCT-NIRF, etc., and/or for employing one or more additional features discussed herein, including, but not limited to, artificial intelligence processes (e.g., machine or deep learning, residual learning, artificial intelligence (“AI”) co-registration, tissue detection, tissue characterization, calibration (ex vivo and/or in vivo), sheath detection, etc.) or other process(es) (e.g., co-registration, tissue detection, tissue characterization, calibration (ex vivo and/or in vivo), sheath detection, etc.) and/or related technique(s) or method(s) such as for ophthalmic applications in accordance with one or more aspects of the present disclosure. FIG. 9C shows an exemplary schematic of an OCT-fluorescence imaging system 100″, according to one or more embodiments of the present disclosure. An OCT light source 101 (e.g., with a 1.3 μm) is delivered and split into a reference arm 102 and a sample arm 103 with a deflector or deflected section (e.g., a splitter) 108, creating a reference beam and sample beam, respectively. The reference beam from the OCT light source 101 is reflected by a reference mirror 105 while a sample beam is reflected or scattered from an object (e.g., an object to be examined, an object, a target, a patient, etc.) 106 through a circulator 901, a rotary junction 90 (“RJ”) and a catheter 120. In one or more embodiments, the fiber between the circulator 901 and the reference mirror or reference reflection 105 may be coiled to adjust the length of the reference arm 102 (best seen in FIG. 9C). Optical fibers in the sample arm 103 may be made of double clad fiber (“DCF”). Excitation light for the fluorescence may be directed to the RJ 90 and the catheter 120, and illuminate the object (e.g., an object to be examined, an object, a patient, etc.) 106. The light from the OCT light source 101 may be delivered through the core of DCF while the fluorescence light emitted from the object (e.g., an object to be examined, an object, a target, a patient, etc.) 106 may be collected through the cladding of the DCF. For pullback imaging, the RJ 90 may be moved with a linear stage to achieve helical scanning of the object (e.g., an object to be examined, an object, a target, a patient, etc.) 106. In one or more embodiments, the RJ 90 may include any one or more features of an RJ as discussed herein. Dichroic filters DF1, DF2 may be used to separate excitation light and the rest of fluorescence and OCT lights. For example (and while not limited to this example), in one or more embodiments, DF1 may be a long pass dichroic filter with a cutoff wavelength of ˜1000 nm, and the OCT light, which may be longer than a cutoff wavelength of DF1, may go through the DF1 while fluorescence excitation and emission, which are a shorter wavelength than the cut off, reflect at DF1. In one or more embodiments, for example (and while not limited to this example), DF2 may be a short pass dichroic filter; the excitation wavelength may be shorter than fluorescence emission light such that the excitation light, which has a wavelength shorter than a cutoff wavelength of DF2, may pass through the DF2, and the fluorescence emission light reflect with DF2. In one embodiment, both beams combine at the deflecting section 108 and generate interference patterns. In one or more embodiments, the beams go to the coupler or combiner 903, and the coupler or combiner 903 combines both beams via the circulator 901 and the deflecting section 108, and the combined beams are delivered to one or more detectors (such as the one or more detectors 107; see e.g., the first detector 107 connected to the coupler or combiner 903 in FIG. 9C).

In one or more embodiments, the optical fiber in the catheter 120 operates to rotate inside the catheter 120, and the OCT light and excitation light may be emitted from a side angle of a tip of the catheter 120. After interacting with the object or patient 106, the OCT light may be delivered back to an OCT interferometer (e.g., via the circulator 901 of the sample arm 103), which may include the coupler or combiner 903, and combined with the reference beam (e.g., via the coupler or combiner 903) to generate interference patterns. The output of the interferometer is detected with a first detector 107, wherein the first detector 107 may be photodiodes or multi-array cameras, and then may be recorded to a computer (e.g., to the computer 2, the computer 1200 as shown in FIG. 9C, the computer 1200′, or any other computer discussed herein) through a first data-acquisition unit or board (“DAQ1”).

Simultaneously or at a different time, the fluorescence intensity may be recorded through a second detector 107 (e.g., a photomultiplier) through a second data-acquisition unit or board (“DAQ2”). The OCT signal and fluorescence signal may be then processed by the computer (e.g., to the computer 2, the computer 1200 as shown in FIG. 9C, the computer 1200′, or any other computer discussed herein) to generate an OCT-fluorescence data set 140, which includes or is made of multiple frames of helically scanned data. Each set of frames includes or is made of multiple data elements of co-registered OCT and fluorescence data, which correspond to the rotational angle and pullback position.

In one or more embodiments, any of the systems 2, 20, 100, 100′, 100″, any other system discussed herein, etc. may be used to perform calibration (ex vivo and/or in vivo) and/or sheath detection feature(s) (e.g., estimating calibration and/or sheath detection, performing calibration and/or sheath detection, etc.) to one or more portions or components of the optical probe 124 and/or the catheter 120 of a respective system or of another system.

Detected fluorescence or auto-fluorescence signals may be processed or further processed as discussed in U.S. Pat. App. No. 62/861,888, filed on Jun. 14, 2019, the disclosure of which is incorporated herein by reference in its entirety, and/or as discussed in U.S. patent application Ser. No. 16/368,510, filed Mar. 28, 2019, the disclosure of which is incorporated herein by reference herein in its entirety.

While not limited to such arrangements, configurations, devices or systems, one or more embodiments of the devices, apparatuses, systems, methods, storage mediums, GUI's, etc. discussed herein may be used with an apparatus or system as aforementioned, such as, but not limited to, for example, the system 20, the system 100, the system 100′, the system 100″, the devices, apparatuses, or systems of FIGS. 1A-8B and 9A-17, any other device, apparatus or system discussed herein, etc. and/or may be used with any AI-ready network discussed herein or known to those skilled in the art. In one or more embodiments, one user may perform the method(s) discussed herein. In one or more embodiments, one or more users may perform the method(s) discussed herein. In one or more embodiments, one or more of the computers, CPUs, processors, etc. discussed herein may be used to process, control, update, emphasize, and/or change one or more of the imaging modalities, and/or process the related techniques, functions or methods, or may process the electrical signals as discussed above.

The light source 101 may include a plurality of light sources or may be a single light source. The light source 101 may be a broadband lightsource, and may include one or more of a laser, an organic light emitting diode (OLED), a light emitting diode (LED), a halogen lamp, an incandescent lamp, supercontinuum light source pumped by a laser, and/or a fluorescent lamp. The light source 101 may be any light source that provides light which may then be dispersed to provide light which is then used for imaging, performing control, viewing, changing, emphasizing methods for imaging modalities, constructing or reconstructing image(s) or structure(s), characterizing tissue, performing calibration (ex vivo and/or in vivo), performing sheath detection, and/or any other method discussed herein. The light source 101 may be fiber coupled or may be free space coupled to the other components of the apparatus and/or system 20, 100, 100′, 100″, the devices, apparatuses or systems of FIGS. 1A-8B and 9A-17, or any other embodiment discussed herein. As aforementioned, the light source 101 may be a swept-source (SS) light source.

Additionally or alternatively, the one or more detectors 107 may be a linear array, a charge-coupled device (CCD), a plurality of photodiodes or some other method of converting the light into an electrical signal. The detector(s) 107 may include an analog to digital converter (ADC). The one or more detectors may be detectors having structure as shown in one or more of FIGS. 1A-8B and 9A-17 and as discussed herein.

In accordance with one or more aspects of the present disclosure, one or more methods for performing imaging are provided herein. FIG. 10 illustrates a flow chart of at least one embodiment of a method for performing imaging. The method(s) may include one or more of the following: (i) splitting or dividing light into a first light and a second reference light (see step S4000 in FIG. 10); (ii) receiving reflected or scattered light of the first light after the first light travels along a sample arm and irradiates an object (see step S4001 in FIG. 10); (iii) receiving the second reference light after the second reference light travels along a reference arm and reflects off of a reference reflection (see step S4002 in FIG. 10); and (iv) generating interference light by causing the reflected or scattered light of the first light and the reflected second reference light to interfere with each other (for example, by combining or recombining and then interfering, by interfering, etc.), the interference light generating one or more interference patterns (see step S4003 in FIG. 10). One or more methods may further include using low frequency monitors to update or control high frequency content to improve image quality. For example, one or more embodiments may use multiple imaging modalities, related methods or techniques for same, etc. to achieve improved image quality. In one or more embodiments, an imaging probe may be connected to one or more systems (e.g., the system 20, the system 100, the system 100′, the system 100″, the devices, apparatuses or systems of FIGS. 1A-8B and 9A-17, any other system or apparatus discussed herein, etc.) with a connection member or interface module. For example, when the connection member or interface module is a rotary junction for an imaging probe, the rotary junction may be at least one of: a contact rotary junction, a lenseless rotary junction, a lens-based rotary junction, or other rotary junction known to those skilled in the art. The rotary junction may be a one channel rotary junction or a two channel rotary junction. The rotary junction may be or include any RJ feature(s) discussed herein, including, but not limited to, the features shown in at least FIGS. 9B-9C. In one or more embodiments, the illumination portion of the imaging probe may be separate from the detection portion of the imaging probe. For example, in one or more applications, a probe may refer to the illumination assembly, which includes an illumination fiber (e.g., single mode fiber, a GRIN lens, a spacer and the grating on the polished surface of the spacer, etc.). In one or more embodiments, a scope may refer to the illumination portion which, for example, may be enclosed and protected by a drive cable, a sheath, and detection fibers (e.g., multimode fibers (MMFs)) around the sheath. Grating coverage is optional on the detection fibers (e.g., MMFs) for one or more applications. The illumination portion may be connected to a rotary joint and may be rotating continuously at video rate. In one or more embodiments, the detection portion may include one or more of: a detection fiber, a detector (e.g., the one or more detectors 107, a spectrometer, etc.), the computer 1200, the computer 1200′, the computer 2, any other computer or processor discussed herein, etc. The detection fibers may surround the illumination fiber, and the detection fibers may or may not be covered by a grating, a spacer, a lens, an end of a probe or catheter, etc.

The one or more detectors 107 may transmit the digital or analog signals to a processor or a computer such as, but not limited to, an image processor, a processor or computer 1200, 1200′ (see e.g., FIG. 1B and FIGS. 9A-9C and 11-13), a computer 2 (see e.g., FIG. 1A), any other processor or computer discussed herein, a combination thereof, etc. The image processor may be a dedicated image processor or a general purpose processor that is configured to process images. In at least one embodiment, the computer 1200, 1200′, 2 or any other processor or computer discussed herein may be used in place of, or in addition to, the image processor. In an alternative embodiment, the image processor may include an ADC and receive analog signals from the one or more detectors 107. The image processor may include one or more of a CPU, DSP, FPGA, ASIC, or some other processing circuitry. The image processor may include memory for storing image, data, and instructions. The image processor may generate one or more images based on the information provided by the one or more detectors 107. A computer or processor discussed herein, such as, but not limited to, a processor of the devices, apparatuses, or systems of FIGS. 1A-8B and 9A-17, the computer 1200, the computer 1200′, the computer 2, the image processor, and/or any other processor discussed herein or AI-ready network or neural network discussed herein or known to those skilled in the art, may also include one or more components further discussed herein below (see e.g., FIGS. 11-13).

In at least one embodiment, a console or computer 1200, 1200′, a computer 2, any other computer or processor discussed herein, etc. operates to control motions of the RJ via the motion control unit (MCU) 112 or a motor M, acquires intensity data from the detector(s) in the one or more detectors 107, and displays the scanned image (e.g., on a monitor or screen such as a display, screen or monitor 1209 as shown in the console or computer 1200 of any of FIGS. 1B, 9A-9C, 11, and 13 and/or the console 1200′ of FIGS. 12-13 as further discussed below; the computer 2 of FIG. 1A; any other computer or processor discussed herein; etc.). In one or more embodiments, the MCU 112 or the motor M operates to change a speed of a motor of the RJ and/or of the RJ. The motor may be a stepping or a DC servo motor to control the speed and increase position accuracy (e.g., compared to when not using a motor, compared to when not using an automated or controlled speed and/or position change device, compared to a manual control, etc.).

The output of the one or more components of any of the systems discussed herein may be acquired with the at least one detector 107, e.g., such as, but not limited to, photodiodes, Photomultiplier tube(s) (PMTs), line scan camera(s), or multi-array camera(s). Electrical analog signals obtained from the output of the system 20, 100, 100′, 100″, and/or the detector(s) 107 thereof, and/or from the devices, apparatuses, or systems of FIGS. 1A-8B and 9A-17, are converted to digital signals to be analyzed with a computer, such as, but not limited to, the computer 1200, 1200′. In one or more embodiments, the light source 101 may be a radiation source or a broadband light source that radiates in a broad band of wavelengths. In one or more embodiments, a Fourier analyzer including software and electronics may be used to convert the electrical analog signals into an optical spectrum.

Unless otherwise discussed herein, like numerals indicate like elements. For example, while variations or differences exist between the systems, such as, but not limited to, the system 20, the system 100, the system 100′, the system 100″, or any other device, apparatus or system discussed herein, one or more features thereof may be the same or similar to each other, such as, but not limited to, the light source 101 or other component(s) thereof (e.g., the console 1200, the console 1200′, etc.). Those skilled in the art will appreciate that the light source 101, the motor or MCU 112, the RJ, the at least one detector 107, and/or one or more other elements of the system 100 may operate in the same or similar fashion to those like-numbered elements of one or more other systems, such as, but not limited to, the devices, apparatuses or systems of FIGS. 1A-8B and 9A-17, the system 100′, the system 100″, or any other system discussed herein. Those skilled in the art will appreciate that alternative embodiments of the devices, apparatuses or systems of FIGS. 1A-8B and 9A-17, the system 100′, the system 100″, any other device, apparatus or system discussed herein, etc., and/or one or more like-numbered elements of one of such systems, while having other variations as discussed herein, may operate in the same or similar fashion to the like-numbered elements of any of the other systems (or components thereof) discussed herein. Indeed, while certain differences exist between the system 100 of FIG. 9A and one or more embodiments shown in any of FIGS. 1A-8B and 9B-17, for example, as discussed herein, there are similarities. Likewise, while the console or computer 1200 may be used in one or more systems (e.g., the system 20, the system 100, the system 100′, the system 100″, the devices, apparatuses or systems of any of FIGS. 1A-17, or any other system discussed herein, etc.), one or more other consoles or computers, such as the console or computer 1200′, any other computer or processor discussed herein, etc., may be used additionally or alternatively.

One or more embodiments of the present disclosure may include taking multiple views (e.g., OCT image, ring view, tomo view, anatomical view, etc.), and one or more embodiments may highlight or emphasize NIRF and/or NIRAF. In one or more embodiments, two handles may operate as endpoints that may bound the color extremes of the NIRF and/or NIRAF data in or more embodiments. In addition to the standard tomographic view, the user may select to display multiple longitudinal views. When connected to an angiography system, the Graphical User Interface (GUI) may also display angiography images.

In accordance with one or more aspects of the present disclosure, the aforementioned features are not limited to being displayed or controlled using any particular GUI. In general, the aforementioned imaging modalities may be used in various ways, including with or without one or more features of aforementioned embodiments of a GUI or GUIs. For example, a GUI may show an OCT image with a tool or marker to change the image view as aforementioned even if not presented with a GUI (or with one or more other components of a GUI; in one or more embodiments, the display may be simplified for a user to display set or desired information).

The procedure to select the region of interest and the position of a marker, an angle, a plane, etc., for example, using a touch screen, a GUI (or one or more components of a GUI; in one or more embodiments, the display may be simplified for a user to display the set or desired information), a processor (e.g., processor or computer 2, 1200, 1200′, or any other processor discussed herein) may involve, in one or more embodiments, a single press with a finger and dragging on the area to make the selection or modification. The new orientation and updates to the view may be calculated upon release of a finger, or a pointer. In one or more embodiments, a region of interest and/or a position of the marker may be set or selected automatically using AI features and/or processing features of the present disclosure.

For one or more embodiments using a touch screen, two simultaneous touch points may be used to make a selection or modification, and may update the view based on calculations upon release.

One or more functions may be controlled with one of the imaging modalities, such as the angiography image view or the intravascular image view (e.g., the OCT image view, the IVUS image view, another intravascular imaging modality image view, etc.), to centralize user attention, maintain focus, and allow the user to see all relevant information in a single moment in time.

In one or more embodiments, one imaging modality may be displayed or multiple imaging modalities may be displayed.

One or more procedures may be used in one or more embodiments to select a region of choice or a region of interest for a view. For example, after a single touch is made on a selected area (e.g., by using a touch screen, by using a mouse or other input device to make a selection, etc.), the semi-circle (or other geometric shape used for the designated area) may automatically adjust to the selected region of choice or interest. Two (2) single touch points may operate to connect/draw the region of choice or interest. For example, a user may desire to view calibrated portion(s) or component(s) of the optical probe 124 and/or of the catheter 120, and/or may desire to view the object, sample, or target 106.

There are many ways to compute intensity, viscosity, resolution (including increasing resolution of one or more images), etc., to use one or more imaging modalities, to construct or reconstruct images or structure(s), to detect tissue and/or characterize tissue, to perform calibration (ex vivo and/or in vivo) and/or sheath detection, etc., and/or related methods for same, discussed herein, digital as well as analog. In at least one embodiment, a computer, such as the console or computer 1200, 1200′, may be dedicated to control and monitor the imaging (e.g., OCT, single mode OCT, multimodal OCT, multiple imaging modalities, IVUS imaging modality, another intravascular imaging modality discussed herein or known to those skilled in the art, etc.) devices, systems, methods and/or storage mediums described herein.

The electric signals used for imaging may be sent to one or more processors, such as, but not limited to, a computer or processor 2 (see e.g., FIG. 1A), a computer 1200 (see e.g., FIGS. 1B, 9A-9C, 11, and 13), a computer 1200′ (see e.g., FIGS. 12 and 13), etc. as discussed further below, via cable(s) or wire(s), such as, but not limited to, the cable(s) or wire(s) 113 (see FIG. 11). Additionally or alternatively, the electric signals, as aforementioned, may be processed in one or more embodiments as discussed above by any other computer or processor or components thereof. The computer or processor 2 as shown in FIG. 1A may be used instead of any other computer or processor discussed herein (e.g., computer or processors 1200, 1200′, etc.), and/or the computer or processor 1200, 1200′ may be used instead of any other computer or processor discussed herein (e.g., computer or processor 2). In other words, the computers or processors discussed herein are interchangeable, and may operate to perform any of the multiple imaging modalities feature(s) and method(s) discussed herein, including using, controlling, and changing a GUI or multiple GUI's and/or performing tissue characterization, tissue detection, calibration (ex vivo and/or in vivo) and/or sheath detection, and coregistration.

Various components of a computer system 1200 are provided in FIG. 11. A computer system 1200 may include a central processing unit (“CPU”) 1201, a ROM 1202, a RAM 1203, a communication interface 1205, a hard disk (and/or other storage device) 1204, a screen (or monitor interface) 1209, a keyboard (or input interface; may also include a mouse or other input device in addition to the keyboard) 1210 and a BUS (or “Bus”) or other connection lines (e.g., connection line 1213) between one or more of the aforementioned components (e.g., including but not limited to, being connected to the console, the probe, the imaging apparatus or system, any motor discussed herein, a light source, etc.). In addition, the computer system 1200 may comprise one or more of the aforementioned components. For example, a computer system 1200 may include a CPU 1201, a RAM 1203, an input/output (I/O) interface (such as the communication interface 1205) and a bus (which may include one or more lines 1213 as a communication system between components of the computer system 1200; in one or more embodiments, the computer system 1200 and at least the CPU 1201 thereof may communicate with the one or more aforementioned components of a device or system, such as, but not limited to, an apparatus or system using one or more imaging modalities and related method(s) as discussed herein), and one or more other computer systems 1200 may include one or more combinations of the other aforementioned components (e.g., the one or more lines 1213 of the computer 1200 may connect to other components via line 113). The CPU 1201 is configured to read and perform computer-executable instructions stored in a storage medium. The computer-executable instructions may include those for the performance of the methods and/or calculations described herein. The system 1200 may include one or more additional processors in addition to CPU 1201, and such processors, including the CPU 1201, may be used for tissue or object characterization, diagnosis, evaluation, imaging, construction or reconstruction, calibration (ex vivo and/or in vivo), sheath detection, and/or coregistration. The system 1200 may further include one or more processors connected via a network connection (e.g., via network 1206). The CPU 1201 and any additional processor being used by the system 1200 may be located in the same telecom network or in different telecom networks (e.g., performing feature(s), function(s), technique(s), method(s), etc. discussed herein may be controlled remotely).

The I/O or communication interface 1205 provides communication interfaces to input and output devices, which may include a light source, a spectrometer, a microphone, a communication cable and a network (either wired or wireless), a keyboard 1210, a mouse (see e.g., the mouse 1211 as shown in FIG. 12), a touch screen or screen 1209, a light pen and so on. The communication interface of the computer 1200 may connect to other components discussed herein via line 113 (as diagrammatically shown in FIG. 11). The Monitor interface or screen 1209 provides communication interfaces thereto.

Any methods and/or data of the present disclosure, such as the methods for performing tissue or object characterization, diagnosis, examination, imaging (including, but not limited to, increasing image resolution, performing imaging using one or more imaging modalities, viewing or changing one or more imaging modalities and related methods (and/or option(s) or feature(s)), etc.), tissue detection, calibration (ex vivo and/or in vivo), sheath detection, and/or coregistration (e.g., using AI feature(s) with one or more of same), for example, as discussed herein, may be stored on a computer-readable storage medium. A computer-readable and/or writable storage medium used commonly, such as, but not limited to, one or more of a hard disk (e.g., the hard disk 1204, a magnetic disk, etc.), a flash memory, a CD, an optical disc (e.g., a compact disc (“CD”) a digital versatile disc (“DVD”), a Blu-ray™ disc, etc.), a magneto-optical disk, a random-access memory (“RAM”) (such as the RAM 1203), a DRAM, a read only memory (“ROM”), a storage of distributed computing systems, a memory card, or the like (e.g., other semiconductor memory, such as, but not limited to, a non-volatile memory card, a solid state drive (SSD) (see SSD 1207 in FIG. 12), SRAM, etc.), an optional combination thereof, a server/database, etc. may be used to cause a processor, such as, the processor or CPU 1201 of the aforementioned computer system 1200 to perform the steps of the methods disclosed herein. The computer-readable storage medium may be a non-transitory computer-readable medium, and/or the computer-readable medium may comprise all computer-readable media, with the sole exception being a transitory, propagating signal in one or more embodiments. The computer-readable storage medium may include media that store information for predetermined, limited, or short period(s) of time and/or only in the presence of power, such as, but not limited to Random Access Memory (RAM), register memory, processor cache(s), etc. Embodiment(s) of the present disclosure may also be realized by a computer of a system or apparatus that reads out and executes computer executable instructions (e.g., one or more programs) recorded on a storage medium (which may also be referred to more fully as a “non-transitory computer-readable storage medium”) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s).

In accordance with at least one aspect of the present disclosure, the methods, systems, and computer-readable storage mediums related to the processors, such as, but not limited to, the processor of the aforementioned computer 1200, etc., as described above may be achieved utilizing suitable hardware, such as that illustrated in the figures. Functionality of one or more aspects of the present disclosure may be achieved utilizing suitable hardware, such as that illustrated in FIG. 11. Such hardware may be implemented utilizing any of the known technologies, such as standard digital circuitry, any of the known processors that are operable to execute software and/or firmware programs, one or more programmable digital devices or systems, such as programmable read only memories (PROMs), programmable array logic devices (PALs), etc. The CPU 1201 (as shown in FIG. 11), the processor or computer 2 (as shown in FIG. 1A) and/or the computer or processor 1200′ (as shown in FIG. 12) may also include and/or be made of one or more microprocessors, nanoprocessors, one or more graphics processing units (“GPUs”; also called a visual processing unit (“VPU”)), one or more Field Programmable Gate Arrays (“FPGAs”), or other types of processing components (e.g., application specific integrated circuit(s) (ASIC)). Still further, the various aspects of the present disclosure may be implemented by way of software and/or firmware program(s) that may be stored on suitable storage medium (e.g., computer-readable storage medium, hard drive, etc.) or media (such as floppy disk(s), memory chip(s), etc.) for transportability and/or distribution. The computer may include a network of separate computers or separate processors to read out and execute the computer executable instructions. The computer executable instructions may be provided to the computer, for example, from a network or the storage medium. The computers or processors (e.g., 2, 1200, 1200′, etc.) may include the aforementioned CPU structure, or may be connected to such CPU structure for communication therewith.

As aforementioned, hardware structure of an alternative embodiment of a computer or console 1200′ is shown in FIG. 12 (see also, FIG. 13). The computer 1200′ includes a central processing unit (CPU) 1201, a graphical processing unit (GPU) 1215, a random access memory (RAM) 1203, a network interface device 1212, an operation interface 1214 such as a universal serial bus (USB) and a memory such as a hard disk drive or a solid state drive (SSD) 1207. The computer or console 1200′ may include a display 1209. The computer 1200′ may connect with a motor, a console, or any other component of the device(s) or system(s) discussed herein via the operation interface 1214 or the network interface 1212 (e.g., via a cable or fiber, such as the cable or fiber 113 as similarly shown in FIG. 11). A computer, such as the computer 1200′, may include a motor or motion control unit (MCU) in one or more embodiments. The operation interface 1214 is connected with an operation unit such as a mouse device 1211, a keyboard 1210 or a touch panel device. The computer 1200′ may include two or more of each component.

At least one computer program is stored in the SSD 1207, and the CPU 1201 loads the at least one program onto the RAM 1203, and executes the instructions in the at least one program to perform one or more processes described herein, as well as the basic input, output, calculation, memory writing and memory reading processes.

The computer, such as the computer 2, the computer 1200, 1200′, (or other component(s) such as, but not limited to, the CPU, etc.), etc. may communicate with a motion control unit (MCU), an interferometer, a spectrometer, a detector, etc. to perform imaging, and may reconstruct an image from the acquired intensity data. The monitor or display 1209 displays the reconstructed image, and may display other information about the imaging condition or about an object to be imaged. The monitor 1209 also provides a graphical user interface for a user to operate any system discussed herein. An operation signal is input from the operation unit (e.g., such as, but not limited to, a mouse device 1211, a keyboard 1210, a touch panel device, etc.) into the operation interface 1214 in the computer 1200′, and corresponding to the operation signal the computer 1200′ instructs any system discussed herein to set or change the imaging condition (e.g., improving resolution of an image or images), and to start or end the imaging. A light or laser source and a spectrometer and/or detector may have interfaces to communicate with the computers 1200, 1200′ to send and receive the status information and the control signals.

As shown in FIG. 13, one or more processors or computers 1200, 1200′ (or any other processor discussed herein) may be part of a system in which the one or more processors or computers 1200, 1200′ (or any other processor discussed herein) communicate with other devices (e.g., a database 1603, a memory 1602 (which may be used with or replaced by any other type of memory discussed herein or known to those skilled in the art), an input device 1600, an output device 1601, etc.). In one or more embodiments, one or more models may have been trained previously and stored in one or more locations, such as, but not limited to, the memory 1602, the database 1603, etc. In one or more embodiments, it is possible that one or more models and/or data discussed herein (e.g., training data, testing data, validation data, imaging data, etc.) may be input or loaded via a device, such as the input device 1600. In one or more embodiments, a user may employ an input device 1600 (which may be a separate computer or processor, a keyboard such as the keyboard 1210, a mouse such as the mouse 1211, a microphone, a screen or display 1209 (e.g., a touch screen or display), or any other input device known to those skilled in the art). In one or more system embodiments, an input device 1600 may not be used (e.g., where user interaction is eliminated by one or more artificial intelligence features discussed herein). In one or more system embodiments, the output device 1601 may receive one or more outputs discussed herein to perform the marker detection, the coregistration, the calibration (ex vivo and/or in vivo), the sheath detection, and/or any other process discussed herein. In one or more system embodiments, the database 1603 and/or the memory 1602 may have outputted information (e.g., trained model(s), detected marker information, image data, test data, validation data, training data, coregistration result(s), calibration (ex vivo and/or in vivo) and/or sheath detection result(s), segmentation model information, object detection/regression model information, combination model information, etc.) stored therein. That said, one or more embodiments may include several types of data stores, memory, storage media, etc. as discussed above, and such storage media, memory, data stores, etc. may be stored locally or remotely.

Additionally, unless otherwise specified, the term “subset” of a corresponding set does not necessarily represent a proper subset and may be equal to the corresponding set.

While one or more embodiments of the present disclosure include various details regarding a neural network model architecture and optimization approach, in one or more embodiments, any other model architecture, machine learning algorithm, or optimization approach may be employed. One or more embodiments may utilize hyper-parameter combination(s). One or more embodiments may employ data capture, selection, annotation as well as model evaluation (e.g., computation of loss and validation metrics) since data may be domain and application specific. In one or more embodiments, the model architecture may be modified and optimized to address a variety of computer visions issues (discussed below).

One or more embodiments of the present disclosure may automatically detect (predict a spatial location of) a radiodense OCT marker in a time series of X-ray images to co-register the X-ray images with the corresponding OCT images (at least one example of a reference point of two different coordinate systems). One or more embodiments may use deep (recurrent) convolutional neural network(s), which may improve marker detection, tissue detection, tissue characterization, calibration (ex vivo and/or in vivo) and/or sheath characterization/detection/performance, and image co-registration significantly. One or more embodiments may employ segmentation and/or object/keypoint detection architectures to solve one or more computer vision issues in other domain areas in one or more applications. One or more embodiments employ several novel materials and methods to solve one or more computer vision or other issues (e.g., radiodense OCT marker detection in time series of X-ray images, for instance; tissue detection; tissue characterization; calibration (ex vivo and/or in vivo); sheath detection; etc.).

One or more embodiments employ data capture and selection. In one or more embodiments, the data is what makes such an application unique and distinguishes this application from other applications. For example, images may include a radiodense marker that is specifically used in one or more procedures (e.g., added to the OCT capsule, used in catheters/probes with a similar marker to that of an OCT marker, used in catheters/probes with a similar or same marker even in a case where the catheters/probes use an imaging modality different from OCT, etc.) to facilitate computational detection of a marker and/or tissue detection, characterization, validation, calibration (ex vivo and/or in vivo), sheath detection, etc. in one or more images (e.g., X-ray images). One or more embodiments may couple a software device or features (model) to hardware (e.g., an OCT probe, a probe/catheter using an imaging modality different from OCT while using a marker that is the same as or similar to the marker of an OCT probe/catheter, etc.). One or more embodiments may utilize animal data in addition to patient data. Training deep learning may use a large amount of data, which may be difficult to obtain from clinical studies. Inclusion of image data from pre-clinical studies in animals into a training set may improve model performance. Training and evaluation of a model may be highly data dependent (e.g., a way in which frames are selected (e.g., pullback only), split into training/validation/test sets, and grouped into batches as well as the order in which the frames, sets, and/or batches are presented to the model, any other data discussed herein, etc.). In one or more embodiments, such parameters may be more important or significant than some of the model hyper-parameters (e.g., batch size, number of convolution layers, any other hyper-parameter discussed herein, etc.). One or more embodiments may use a collection or collections of user annotations after introduction of a device/apparatus, system, and/or method(s) into a market, and may use post market surveillance, retraining of a model or models with new data collected (e.g., in clinical use), and/or a continuously adaptive algorithm/method(s). In one or more embodiments (and while not limited hereto), an A-line image or images may be input into one or more trained models, and one or more outputs may be an image having the calibration (ex vivo and/or in vivo) completed/detected, having a sheath detected, and/or having one or more dashed or dotted line or outline indicators overlaid on the image (e.g., an indicator may indicate that the calibration was completed successfully or not, an indicator may indicate whether a sheath was detected, an indicator may show alignment of a sheath and a ring mark, etc.).

One or more embodiments may employ data annotation. For example, one or more embodiments may label pixel(s) representing a marker, a sheath, or a tissue detection, characterization, and/or validation as well as pixels representing a blood vessel(s) and/or calibration (in vivo and/or ex vivo) characterization/detection/performance at different phase(s) of a procedure/method (e.g., different levels of contrast due to intravascular contrast agent) of frame(s) acquired during pullback.

One or more embodiments may employ incorporation of prior knowledge. For example, in one or more embodiments, a marker location may be known inside a vessel and/or inside a catheter or probe; a tissue location may be known inside a vessel or other type of target, object, or specimen; a ring mark may be known; sheath (and/or other) portion(s) and/or component(s) of the optical probe 124 and/or the catheter 120 may be known; calibration information (e.g., whether ex vivo and/or in vivo calibration(s) were performed successfully or not) may be known; etc. As such, simultaneous localization of the vessel and marker may be used to improve marker detection, and/or tissue and/or calibration (ex vivo and/or in vivo) and/or sheath detection, characterization, and/or validation. For example, in a case where it is confirmed that the marker of the probe or catheter, or the catheter or probe, is by or near a target area for tissue and/or sheath detection and characterization and/or for calibration (ex vivo and/or in vivo), the integrity of the tissue and/or sheath identification/detection and/or characterization for that target area, and/or the calibration (ex vivo and/or in vivo), is improved or maximized (as compared to a false positive where a tissue or a sheath may be detected in an area where the probe or catheter (or marker thereof) is not located). In one or more embodiments, a marker may move during a pullback inside a vessel, and such prior knowledge may be incorporated into the machine learning algorithm or the loss function.

One or more embodiments employ loss (cost) and evaluation function(s)/metric(s). For example, use of temporal information for model training and evaluation may be used in one or more embodiments. One or more embodiments may evaluate a distance between prediction and ground truth per frame as well as consider a trajectory of predictions across multiple frames of a time series. For example, the calibration (ex vivo and/or in vivo) and/or sheath detection process(es) of the portion(s) or component(s) of the optical probe 124 and/or of the catheter 120 may be evaluated over time using a distance between prediction and ground truth per frame.

### Application of Machine Learning

Application of machine learning may be used in one or more embodiment(s), as discussed in PCT/US2020/051615, filed on Sep. 18, 2020 and published as WO 2021/055837 A9 on Mar. 25, 2021, and as discussed in U.S. patent application Ser. No. 17/761,561, filed on Mar. 17, 2022, the applications and publications of which are incorporated by reference herein in their entireties. For example, at least one embodiment of an overall process of machine learning is shown below:
i. Create a dataset that contains both images and corresponding ground truth labels;
ii. Split the dataset into a training set and a testing set;
iii. Select a model architecture and other hyper-parameters;
iv. Train the model with the training set;
V. Evaluate the trained model with the validation set; and
vi. Repeat iv and v with new dataset(s).

Based on the testing results, steps i and iii may be revisited in one or more embodiments.

One or more models may be used in one or more embodiment(s) to detect and/or characterize a tissue or tissues and/or to detect and/or characterize calibration (ex vivo and/or in vivo) and/or a sheath, such as, but not limited to, the one or more models as discussed in PCT/US2020/051615, filed on Sep. 18, 2020 and published as WO 2021/055837 A9 on Mar. 25, 2021, and as discussed in U.S. patent application Ser. No. 17/761,561, filed on Mar. 17, 2022, the applications and publications of which are incorporated by reference herein in their entireties. For example, one or more embodiments may use a segmentation model, a regression model, a combination thereof, etc.

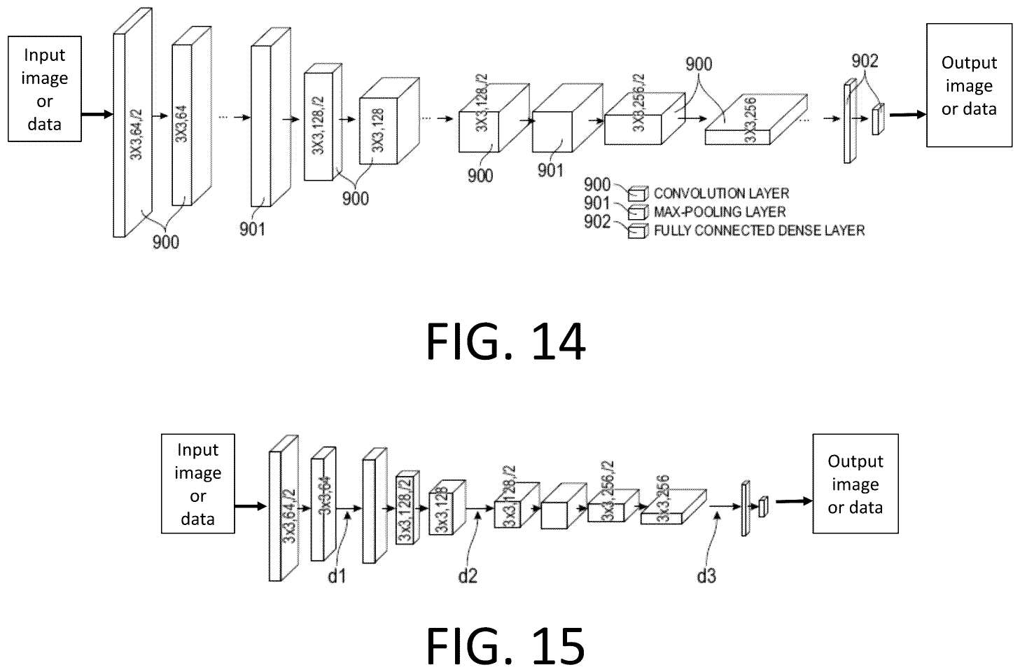

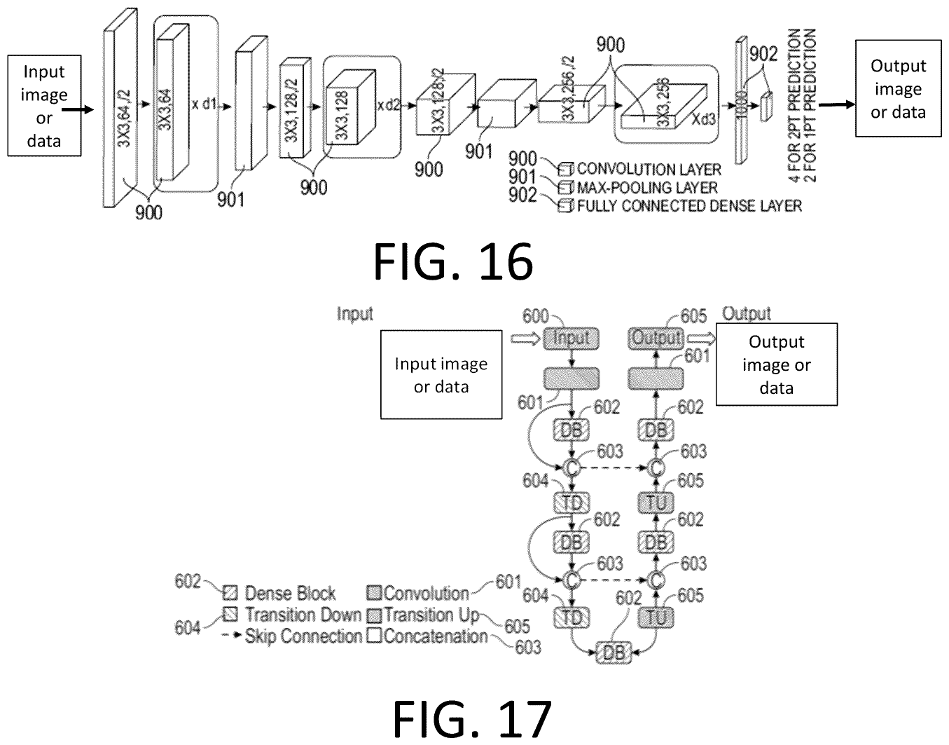

For regression model(s), the input may be the entire image frame or frames, and the output may be the centroid coordinates of radiopaque markers (target marker and stationary marker, if necessary/desired) and/or coordinates of a portion of a catheter or probe to be used in determining the tissue detection and/or characterization and/or used in determining calibrated (ex vivo and/or in vivo) and/or sheath portion(s) or component(s) of the optical probe 124 and/or of the catheter 120. Additionally or alternatively, in one or more embodiments, input may comprise or include an entire image frame or frames (e.g., the aforementioned constructed CVI image or frame), and the output may be data regarding high textured areas formed due to the presence of sharp edges in an A-line or A-lines representing calcium in intravascular images as well as dark homogeneous areas representing lipids in the input image frame or frames (e.g., in the CVI image or frame). As shown diagrammatically in FIGS. 14-16, an example of an input image on the left side of FIGS. 14-16 and a corresponding output image on the right side of FIGS. 14-16 are illustrated for regression model(s). At least one architecture of a regression model is shown in FIG. 14. In at least the embodiment of FIG. 14, the regression model may use a combination of one or more convolution layers 900, one or more max-pooling layers 901, and one or more fully connected dense layers 902. While not limited to the Kernel size, Width/Number of filters (output size), and Stride sizes shown for each layer (e.g., in the left convolution layer of FIG. 14, the Kernel size is “3×3”, the Width/# of filters (output size) is “64”, and the Stride size is “2”). In one or more embodiments, another hyper-parameter search with a fixed optimizer and with a different width may be performed, and at least one embodiment example of a model architecture for a convolutional neural network for this scenario is shown in FIG. 15. One or more embodiments may use one or more features for a regression model as discussed in “Deep Residual Learning for Image Recognition” to Kaiming He, et al., Microsoft Research, Dec. 10, 2015 (https://arxiv.org/pdf/1512.03385.pdf), which is incorporated by reference herein in its entirety. FIG. 16 shows at least a further embodiment example of a created architecture of or for a regression model(s).

Since the output from a segmentation model, in one or more embodiments, is a “probability” of each pixel that may be categorized as a tissue, sheath, and/or calibration (ex vivo and/or in vivo) characterization/identification/determination, post-processing after prediction via the trained segmentation model may be developed to better define, determine, or locate the final coordinate of tissue or sheath location (or a marker location where the marker is a part of the catheter) and/or determine the type and/or characteristics of the tissue or tissues or of the calibration (ex vivo and/or in vivo). One or more embodiments of a semantic segmentation model may be performed using the One-Hundred Layers Tiramisu method discussed in “The One Hundred Layers Tiramisu: Fully Convolutional DenseNets for Semantic Segmentation” to Simon Jegou, et al., Montreal Institute for Learning Algorithms, published Oct. 31, 2017 (https://arxiv.org/pdf/1611.09326.pdf), which is incorporated by reference herein in its entirety. A segmentation model may be used in one or more embodiment, for example, as shown in FIG. 17. At least one embodiment may utilize an input 600 as shown to obtain an output 605 of at least one embodiment of a segmentation model method. For example, by applying the One-Hundred Layers Tiramisu method(s), one or more features, such as, but not limited to, convolution 601, concatenation 603, transition up 605, transition down 604, dense block 602, etc., may be employed by slicing the training data set. While not limited to only or by only these embodiment examples, in one or more embodiments, a slicing size may be one or more of the following: 100×100, 224×224, 512×512, and, in one or more of the experiments performed, a slicing size of 224×224 performed the best. A batch size (of images in a batch) may be one or more of the following: 2, 4, 8, 16, and, from the one or more experiments performed, a bigger batch size typically performs better (e.g., with greater accuracy). In one or more embodiments, 16 images/batch may be used. The optimization of all of these hyper-parameters depends on the size of the available data set as well as the available computer/computing resources; thus, once more data is available, different hyper-parameter values may be chosen. Additionally, in one or more embodiments, steps/epoch may be 100, and the epochs may be greater than (>) 1000. In one or more embodiments, a convolutional autoencoder (CAE) may be used.

In one or more embodiments, hyper-parameters may include, but are not limited to, one or more of the following: Depth (i.e., # of layers), Width (i.e., # of filters), Batch size (i.e., # of training images/step): May be >4 in one or more embodiments, Learning rate (i.e., a hyper-parameter that controls how fast the weights of a neural network (the coefficients of regression model) are adjusted with respect the loss gradient), Dropout (i.e., % of neurons (filters) that are dropped at each layer), and/or Optimizer: for example, Adam optimizer or Stochastic gradient descent (SGD) optimizer. In one or more embodiments, other hyper-parameters may be fixed or constant values, such as, but not limited to, for example, one or more of the following: Input size (e.g., 1024 pixel×1024 pixel, 512 pixel×512 pixel, another preset or predetermined number or value set, etc.), Epochs: 100, 200, 300, 400, 500, another preset or predetermined number, etc. (for additional training, iteration may be set as 3000 or higher), and/or Number of models trained with different hyper-parameter configurations (e.g., 10, 20, another preset or predetermined number, etc.).

One or more features discussed herein may be determined using a convolutional auto-encoder, Gaussian filters, Haralick features, and/or thickness or shape of the sample or object (e.g., the tissue or tissues, a sheath, a specimen, a patient, a target in the patient, calibrated (ex vivo and/or in vivo) and/or sheath portion(s) or component(s) of the optical probe 124 and/or of the catheter 120, etc.).

One or more embodiments of the present disclosure may use machine learning to determine marker, tissue, sheath, or calibration (ex vivo and/or in vivo) location; to determine, detect, or evaluate tissue and/or sheath type(s) and/or characteristic(s); to determine, detect, evaluate, or perform calibration (ex vivo and/or in vivo) characteristic(s); to perform coregistration; and/or to perform any other feature discussed herein. Machine learning (ML) is a field of computer science that gives processors the ability to learn, via artificial intelligence. Machine learning may involve one or more algorithms that allow processors or computers to learn from examples and to make predictions for new unseen data points. In one or more embodiments, such one or more algorithms may be stored as software or one or more programs in at least one memory or storage medium, and the software or one or more programs allow a processor or computer to carry out operation(s) of the processes described in the present disclosure.

Similarly, the present disclosure and/or one or more components of devices, systems, and storage mediums, and/or methods, thereof also may be used in conjunction with optical coherence tomography probes. Such probes include, but are not limited to, the OCT imaging systems disclosed in U.S. Pat. Nos. 6,763,261; 7,366,376; 7,843,572; 7,872,759; 8,289,522; 8,676,013; 8,928,889; 9,087,368; 9,557,154; 10,912,462; 9,795,301; and U.S. Pat. No. 9,332,942 to Tearney et al., and U.S. Pat. Pub. Nos. 2014/0276011 and 2017/0135584; and WO 2016/015052 to Tearney et al., and arrangements and methods of facilitating photoluminescence imaging, such as those disclosed in U.S. Pat. No. 7,889,348 to Tearney et al., as well as the disclosures directed to multimodality imaging disclosed in U.S. Pat. No. 9,332,942, and U.S. Patent Publication Nos. 2010/0092389, 2011/0292400, 2012/0101374, 2016/0228097, 2018/0045501 and 2018/0003481, and WO 2016/144878, each of which patents and patent publications are incorporated by reference herein in their entireties. As aforementioned, any feature or aspect of the present disclosure may be used with OCT imaging systems, apparatuses, methods, storage mediums or other aspects or features as discussed in U.S. patent application Ser. No. 16/414,222, filed on May 16, 2019 and published on Dec. 12, 2019 as U.S. Pat. Pub. No. 2019/0374109, the entire disclosure of which is incorporated by reference herein in its entirety.

The present disclosure and/or one or more components of devices, systems, and storage mediums, and/or methods, thereof also may be used in conjunction with OCT imaging systems and/or catheters and catheter systems, such as, but not limited to, those disclosed in U.S. Pat. Nos. 9,869,828; 10,323,926; 10,558,001; 10,601,173; 10,606,064; 10,743,749; 10,884,199; 10,895,692; and 11,175,126 as well as U.S. Patent Publication Nos. 2019/0254506; 2020/0390323; 2021/0121132; 2021/0174125; 2022/0040454; 2022/0044428, and WO2021/055837, each of which patents and patent publications are incorporated by reference herein in their entireties.

Further, the present disclosure and/or one or more components of devices, systems, and storage mediums, and/or methods, thereof also may be used in conjunction with continuum robotic systems and catheters, such as, but not limited to, those described in U.S. Patent Publication Nos. 2019/0105468; 2021/0369085; 2020/0375682; 2021/0121162; 2021/0121051; and 2022-0040450, each of which patents and/or patent publications are incorporated by reference herein in their entireties.

Although the disclosure herein has been described with reference to particular embodiments, it is to be understood that these embodiments are merely illustrative of the principles and applications of the present disclosure (and are not limited thereto). It is therefore to be understood that numerous modifications may be made to the illustrative embodiments and that other arrangements may be devised without departing from the spirit and scope of the present disclosure. The scope of the following claims is to be accorded the broadest interpretation so as to encompass all such modifications and equivalent structures and functions.

1. An apparatus for calibrating a catheter or probe, the apparatus comprising:
one or more processors that operate to:
obtain or receive one or more images or one or more A-line images; and
automatically calibrate the catheter or probe using an external calibration before the catheter or probe is inserted into a target, sample, or object and an in vivo calibration after the catheter or probe is inserted into the target, sample, or object, wherein:
for the external calibration, the one or more processors operate to detect one or more skeletons or portions of a sheath of the catheter or probe and determine whether the skeletons or portions of the sheath are in a target or set position, and
for the in vivo calibration, the one or more processors operate to detect blood or a blood border position to automatically locate a position of the one or more skeletons or portions of the sheath and then adjust or re-adjust the image or the A-line image and calibrate or re-calibrate the catheter or probe to reduce or remove any effects caused by in vivo or environmental changes.

2. The apparatus of claim 1, wherein the one or more processors further operate to calibrate or re-calibrate the catheter or probe even in a case where high noise is present.

3. The apparatus of claim 1, wherein the catheter or probe uses one or more imaging modalities, where the one or more imaging modalities include one or more of the following: Optical Coherence Tomography (OCT), single modality OCT, multi-modality OCT, swept source OCT, optical frequency domain imaging (OFDI), intravascular ultrasound (IVUS), another lumen image(s) modality, near-infrared spectroscopy (NIRS), near-infrared fluorescence (NIRF), near-infrared auto-fluorescence (NIRAF), near-infrared, fluorescence, and/or an intravascular imaging modality.

4. The apparatus of claim 1, wherein, for the external calibration, the one or more processors further operate to path match a reference path/arm of the catheter or probe and a sample path/arm of the catheter or probe by moving a delay line, or a motorized delay line, to change the reference path/arm so that a ring mark or a mark of a set or predetermined size and shape matches or substantially matches the sheath in at least one of the one or more images or A-line images.

5. The apparatus of claim 4, wherein the one or more processors further operate to one or more of the following:
(i) crop an image of the one or more images or A-line images to an area of interest;
(ii) filter the image;
(iii) binarize the image;
(iv) detect rectangles or shapes that include the skeletons or portions of binary objects of the sheath;
(v) select the skeletons or portions having a height >h1 and <h2;
(vi) find a plurality of differences of middle lines (RL) of the rectangles or shapes to a fixed line (GL), where the difference represents or corresponds to the rectangles or shapes of the binary objects of the sheath;
(vii) determine whether a 1^{st} (RL−GL) difference of the plurality of differences is <4 and the rest of the (RL−GL) differences <than 21 through 25 or is between 25 and 21; and/or
(viii) determine that the catheter or probe is externally calibrated in a case where the 1^{st} (RL−GL) difference of the plurality of differences is <4 and the rest of the (RL−GL) differences <than 21 through 25 or is between 25 and 21 or, in a case where the catheter or probe is not yet externally calibrated, then move the delay line, or the motorized delay line, to d to −d and repeat steps (i) through (vii) for a new image or A-line image that is acquired.

6. The apparatus of claim 5, wherein one or more of the following:
(i) the one or more images or A-line images are in polar coordinates;
(ii) the image of the one or more images or A-line images is binarized using bilateral filtering and/or non-linear smoothing;
(iii) the image of the one or more images or A-line images is binarized using bilateral filtering and/or non-linear smoothing, wherein the bilateral filtering is performed using intensity differences of one or more pixels, which result in edge maintenance simultaneously with noise reduction;
(iv) using one or more convolutions, a weighted average of neighborhood pixel intensities replace an intensity of a central pixel of a mask;
(v) the one or more processors further operate to detect a border or borders of cross sections of the one or more images or the A-line images and/or to perform segmentation procedure(s) of the A-line cross-section(s);
(vi) an image I of the one or more images or A-line images is binarized using bilateral filtering and/or non-linear smoothing, wherein a bilateral filter for the image I, and a window mask W is defined as:
$${I}^{′} ( x ) = \frac{1}{{W}_{p}} {\sum }_{{x}_{i} ∈ W} I ( {x}_{i} ) {f}_{r} ( ‖ I ( {x}_{i} ) - I ( x ) ‖ ) {g}_{s} ( ‖ {x}_{i} - x ‖ ) ,$$
having a normalization factor Wₚ: Wₚ=Σₓ_{ᵢ}_{∈w} fᵣ(∥I(xᵢ)−I(x)∥)gₛ(∥xᵢ−x∥), where x are coordinates of the mask's central pixel and the parameters fᵣ and gₛ are a Gaussian kernel for smoothing differences in intensities and a spatial Gaussian kernel for smoothing differences in coordinates; and/or
(vii) the one or more processors further operate to perform bilateral filtering for an image I, and a window mask W is defined as:
$${I}^{′} ( x ) = \frac{1}{{W}_{p}} {\sum }_{{x}_{i} ∈ W} I ( {x}_{i} ) {f}_{r} ( ‖ I ( {x}_{i} ) - I ( x ) ‖ ) {g}_{s} ( ‖ {x}_{i} - x ‖ ) ,$$
having a normalization factor Wₚ: Wₚ=Σₓ_{ᵢ}_{∈w} fᵣ(∥I(xᵢ)−I(x)∥)gₛ(∥xᵢ−x∥), where x are the coordinates of a central pixel of the mask and the parameters fᵣ and gₛ are a Gaussian kernel for smoothing differences in intensities and a spatial Gaussian kernel for smoothing differences in coordinates.

7. The apparatus of claim 6, wherein one or more of the following:
(i) the image, the image I, or the image I′ of the one or more images or A-line images is automatically thresholded using Otsu's thresholding method, and one or more binary objects are revealed or detected;
(ii) the one or more processors further operate to apply a filtering technique or bilateral filtering and delete the catheter or probe from the one or more images or A-line images;
(iii) the one or more processors further operate to apply Otsu's automatic thresholding;
(iv) to automatically threshold cross sections of the one or more images or the A-line images or to automatically threshold the image, the image I, or the image I′ of the one or more images or A-line images, a threshold Thrₒₜₛᵤ for the image I′ is calculated using Otsu's thresholding method, and the pixels of the image I′ that are smaller than Thrₒₜₛᵤ are set to zero value, where the result is a binary image with a guide wire being represented by the zero objects and one or more binary objects are revealed or detected;
(v) for all of the revealed or detected binary objects, rectangles or geometric shapes that include each revealed or detected binary object are calculated;
(vi) for all of the revealed or detected binary objects, rectangles or geometric shapes that include each revealed or detected binary object are calculated, and for the rectangles or geometric shapes having a height between 3 (h1) and 100 (h2) pixels, the one or more processors further operate to calculate a middle line, RL, for each rectangle or geometric shape and to find an absolute difference of a respective middle line, RL, of each rectangle or geometric shape to a fixed line, GL;
(vii) for all of the revealed or detected binary objects, rectangles or geometric shapes that include each revealed or detected binary object are calculated, and for the rectangles or geometric shapes having a height between 3 (h1) and 100 (h2) pixels, the one or more processors further operate to calculate a middle line, RL, for each rectangle or geometric shape and to find an absolute difference of a respective middle line, RL, of each rectangle or geometric shape to a fixed line, GL, where GL represents a line of a binary sheath of the rectangles or geometric shapes in the catheter or probe that is calibrated; and/or
(viii) the one or more processors further operate to select the rectangles, geometric shapes, or boxes by selecting the skeletons or portions of the sheath of the catheter or probe having height >h1 and <h2.

8. The apparatus of claim 1, wherein one or more of the following:
(i) for the in vivo calibration, the one or more processors further operate to perform imaging alignment by detecting the sheath of the probe or catheter by detecting the blood or the blood border position and using the sheath as a zero point for measurements to reduce or remove error(s) or the effects caused by changing environmental materials and/or condition(s);
(ii) once the image or the catheter or probe is externally calibrated, then the catheter or probe is inserted into the target, object, or sample;
(iii) once the image or the catheter or probe is externally calibrated and the catheter or probe is inserted into the target, object, or sample, then the delay line, or the motorized delay line, is not moved, and any calibration error is corrected by adjusting the image of the one or more images or A-line images; and/or
(iv) the one or more processors further operate to one or more of the following: (1) acquire one image or A-line image of the one or more images or A-line images; (2) apply bilateral filtering and Otsu's thresholding method to one image or A-line image of the one or more images or A-line images; (3) detect a bottom line area of a biggest detected object or binary object, where the bottom line area corresponds to an outer sheath boundary for the catheter or the probe; and/or (4) shift one image or A-line image of the one or more images or A-line images such that a detected outer sheath boundary matches or substantially matches a zero point or position which corresponds to a ring mark or a mark of a set or predetermined size and shape or which corresponds to an outer surface of a sheath of the catheter or probe, where all distances are measured outward from the zero point or position.

9. The apparatus of claim 1, further comprising:
an interference optical system that operates to: (i) receive and divide light from a light source into a first light with which the target, object, or sample is to be irradiated and which travels along a sample arm of the interference optical system and a second reference light, (ii) send the second reference light along a reference arm of the interference optical system for reflection off of a reference reflection of the interference optical system, and (iii) generate interference light by causing reflected or scattered light of the first light with which the target, object, or sample has been irradiated and the reflected second reference light to combine or recombine, and to interfere, with each other, the interference light generating one or more interference patterns; and
one or more detectors that operate to continuously acquire the interference light and/or the one or more interference patterns to measure the interference or the one or more interference patterns between the combined or recombined light to obtain data for one or more imaging modalities,
wherein: (i) a wavelength of the first light is shorter than a wavelength of the reflected or scattered light and/or the generated interference light, and/or (ii) the interference optical system or the catheter or probe includes a double clad fiber.

10. The apparatus of claim 9, further comprising one or more of the following:
(i) the light source that operates to produce the light;
(ii) the light source that operates to produce the light, the light source producing the light to operate as an excitation laser or light having a wavelength of 400 nm-900 nm or 635 nm; and/or
(iii) the light source that operates to produce the light, the light source producing the light as an excitation laser or light and coupling the excitation laser or light into the interference optical system, the optical probe, and/or one or more components of the optical probe and/or of the catheter.

11. The apparatus of claim 1, wherein the one or more processors further operate to one or more of the following:
(i) perform a pullback of the catheter or probe and/or obtain or receive the one or more images or the one or more A-line images of one or more imaging modalities from the pullback of the catheter or probe; and/or
(ii) display the one or more images or the one or more A-line images on a display, store the one or more images or the one or more A-line images in a memory, or use the one or more images or the one or more A-line images to train one or more models or AI-networks to (a) perform the external calibration and/or the in vivo calibration and/or (b) automatically obtain the one or more images or the one or more A-line images of the one or more imaging modalities.

12. The apparatus of claim 11, wherein, in a case where the one or more processors have trained one or more models or AI-networks, one or more of the following:
(i) the trained model is one or a combination of the following: a neural net model or neural network model, a deep convolutional neural network model, a recurrent neural network model with long short-term memory that can take temporal relationships across images or frames into account, a generative adversarial network (GAN) model, a consistent generative adversarial network (cGAN) model, a three cycle-consistent generative adversarial network (3cGAN) model, a model that can take temporal relationships across images or frames into account, a model that can take temporal relationships into account including tissue location(s) and/or calibration location(s) during pullback in a vessel and/or including tissue and/or calibration characterization data during pullback in a vessel, a model that can use prior knowledge about a procedure and incorporate the prior knowledge into the machine learning algorithm or a loss function, a model using feature pyramid(s) that can take different image resolutions into account, and/or a model using residual learning technique(s), a segmentation model, a segmentation model with post-processing, a model with pre-processing, a model with post-processing, a segmentation model with pre-processing, a deep learning or machine learning model, a semantic segmentation model or classification model, an object detection or regression model, an object detection or regression model with pre-processing or post-processing, a combination of a semantic segmentation model and an object detection or regression model, a model using repeated segmentation model technique(s), a model using feature pyramid(s), a genetic algorithm that operates to breed multiple models for improved performance, and/or a model using repeated object detection or regression model technique(s); and/or
(ii) the one or more processors further operate to use one or more neural networks or convolutional neural networks to one or more of: load a trained model of images or A-line images; perform external and/or in vivo calibration on the catheter or probe; determine whether the external and/or in vivo calibration is/are accurate or correct; determine one or more of the characteristics of one or more objects, targets, or samples in the one or more images or the one or more A-line images; identify or detect the one or more objects, targets, or samples; overlay data on at least one of the one or more images or A-line images to show location(s) of intravascular image(s), the calibrated catheter or probe, and/or the objects, targets, or samples; incorporate image processing and machine learning (ML) or deep learning to automatically identify and locate portions or components of the catheter or probe and/or to perform external and/or in vivo calibration of the catheter or probe; incorporate image processing and machine learning (ML) or deep learning to automatically identify and locate the one or more objects, targets, or samples; display the results for the external and/or in vivo calibration, the identification/detection or characterization on a display; and/or acquire or receive image data during the pullback operation of the catheter or the optical probe.

13. The apparatus of claim 1, wherein the one or more components of the catheter or probe include or comprise a double clad fiber.

14. A method for externally calibrating and in vivo calibrating a catheter or probe of an apparatus, the method comprising:
obtaining or receiving one or more images or one or more A-line images;
automatically calibrating the catheter or probe using an external calibration, before the catheter or probe is inserted into a target, sample, or object, by detecting one or more skeletons or portions of a sheath of the catheter or probe and determining whether the skeletons or portions of the sheath are in a target or set position; and
automatically calibrating the catheter or probe using an in vivo calibration, after the catheter or probe is inserted into the target, sample, or object, by detecting blood or a blood border position to automatically locate a position of the one or more skeletons or portions of the sheath and then adjusting or re-adjusting the image or the A-line image and calibrating or re-calibrating the catheter or probe to reduce or remove any effects caused by in vivo or environmental changes.

15. The method of claim 14, wherein the obtaining or receiving step, the automatically calibrating the catheter or probe using an external calibration step, and the automatically calibrating the catheter or probe using an in vivo calibration step are performed using or via one or more processors of the apparatus.

16. The method of claim 14, further comprising calibrating or re-calibrating the catheter or probe even in a case where high noise is present.

17. The method of claim 14, wherein the catheter or probe uses one or more imaging modalities, where the one or more imaging modalities include one or more of the following: Optical Coherence Tomography (OCT), single modality OCT, multi-modality OCT, swept source OCT, optical frequency domain imaging (OFDI), intravascular ultrasound (IVUS), another lumen image(s) modality, near-infrared spectroscopy (NIRS), near-infrared fluorescence (NIRF), near-infrared auto-fluorescence (NIRAF), near-infrared, fluorescence, and/or an intravascular imaging modality.

18. The method of claim 14, further comprising, for the external calibration, path matching a reference path/arm of the catheter or probe and a sample path/arm of the catheter or probe by moving a delay line, or a motorized delay line, to change the reference path/arm so that a ring mark or a mark of a set or predetermined size and shape matches or substantially matches the sheath in at least one of the one or more images or A-line images.

19. The method of claim 18, further comprising one or more of the following:
(i) cropping an image of the one or more images or A-line images to an area of interest;
(ii) filtering the image;
(iii) binarizing the image;
(iv) detecting rectangles or shapes that include the skeletons or portions of binary objects of the sheath;
(v) selecting the skeletons or portions having a height >h1 and <h2;
(vi) finding a plurality of differences of middle lines (RL) of the rectangles or shapes to a fixed line (GL), where the difference represents or corresponds to the rectangles or shapes of the binary objects of the sheath;
(vii) determining whether a 1^{st} (RL−GL) difference of the plurality of differences is <4 and the rest of the (RL−GL) differences <than 21 through 25 or is between 25 and 21; and/or
(viii) determining that the catheter or probe is externally calibrated in a case where the 1^{st} (RL−GL) difference of the plurality of differences is <4 and the rest of the (RL−GL) differences <than 21 through 25 or is between 25 and 21 or, in a case where the catheter or probe is not yet externally calibrated, then moving the delay line, or the motorized delay line, to d to −d and repeating steps (i) through (vii) for a new image or A-line image that is acquired.

20. The method of claim 19, further comprising one or more of the following:
(i) obtaining or receiving the one or more images or A-line images being in polar coordinates;
(ii) binarizing the image of the one or more images or A-line images using bilateral filtering and/or non-linear smoothing;
(iii) binarizing the image of the one or more images or A-line images using bilateral filtering and/or non-linear smoothing, wherein the bilateral filtering is performed using intensity differences of one or more pixels, which result in edge maintenance simultaneously with noise reduction;
(iv) using one or more convolutions, replacing an intensity of a central pixel of a mask with a weighted average of neighborhood pixel intensities;
(v) detecting a border or borders of cross sections of the one or more images or the A-line images and/or performing segmentation procedure(s) of the A-line cross-section(s);
(vi) binarizing an image I of the one or more images or A-line images using bilateral filtering and/or non-linear smoothing, wherein a bilateral filter for the image I and a window mask W is defined as:
$${I}^{′} ( x ) = \frac{1}{{W}_{p}} {\sum }_{{x}_{i} ∈ W} I ( {x}_{i} ) {f}_{r} ( ‖ I ( {x}_{i} ) - I ( x ) ‖ ) {g}_{s} ( ‖ {x}_{i} - x ‖ ) ,$$
having a normalization factor Wₚ: Wₚ=Σₓ_{ᵢ}_{∈w} fᵣ(∥I(xᵢ)−I(x)∥)gₛ(∥xᵢ−x∥), where x are coordinates of the mask's central pixel and the parameters fᵣ and gₛ are a Gaussian kernel for smoothing differences in intensities and a spatial Gaussian kernel for smoothing differences in coordinates; and/or
(vii) performing bilateral filtering, where a bilateral filter for the image I and a window mask W is defined as:
$${I}^{′} ( x ) = \frac{1}{{W}_{p}} {\sum }_{{x}_{i} ∈ W} I ( {x}_{i} ) {f}_{r} ( ‖ I ( {x}_{i} ) - I ( x ) ‖ ) {g}_{s} ( ‖ {x}_{i} - x ‖ ) ,$$
having a normalization factor Wₚ: Wₚ=Σₓ_{ᵢ}_{∈w} fᵣ(∥I(xᵢ)−I(x)∥)gₛ(∥xᵢ−x∥), where x are the coordinates of a central pixel of the mask and the parameters fᵣ and gₛ are a Gaussian kernel for smoothing differences in intensities and a spatial Gaussian kernel for smoothing differences in coordinates.

21. The method of claim 20, further comprising one or more of the following:
(i) automatically thresholding the image, the image I, or the image I′ of the one or more images or A-line images using Otsu's thresholding method, and revealing or detecting one or more binary objects;
(ii) applying a filtering technique or bilateral filtering and deleting the catheter or probe from the one or more images or A-line images;
(iii) applying Otsu's automatic thresholding;
(iv) to automatically threshold cross sections of the one or more images or the A-line images or to automatically threshold the image, the image I, or the image I′ of the one or more images or A-line images, calculating a threshold Thrₒₜₛᵤ for the image I′ using Otsu's thresholding method, and setting the pixels of the image I′ that are smaller than Thrₒₜₛᵤ to zero value, where the result is a binary image with a guide wire being represented by the zero objects and one or more binary objects are revealed or detected;
(v) for all of the revealed or detected binary objects, calculating rectangles or geometric shapes that include each revealed or detected binary object;
(vi) for all of the revealed or detected binary objects, calculating rectangles or geometric shapes that include each revealed or detected binary object, and for the rectangles or geometric shapes having a height between 3 (h1) and 100 (h2) pixels, calculating a middle line, RL, for each rectangle or geometric shape and finding an absolute difference of a respective middle line, RL, of each rectangle or geometric shape to a fixed line, GL;
(vii) for all of the revealed or detected binary objects, calculating rectangles or geometric shapes that include each revealed or detected binary object, and for the rectangles or geometric shapes having a height between 3 (h1) and 100 (h2) pixels, calculating a middle line, RL, for each rectangle or geometric shape and finding an absolute difference of a respective middle line, RL, of each rectangle or geometric shape to a fixed line, GL, where GL represents a line of a binary sheath of the rectangles or geometric shapes in the catheter or probe that is calibrated; and/or
(viii) selecting the rectangles, geometric shapes, or boxes by selecting the skeletons or portions of the sheath of the catheter or probe having height >h1 and <h2.

22. The method of claim 14, further comprising one or more of the following:
(i) for the in vivo calibration, performing imaging alignment by detecting the sheath of the probe or catheter by detecting the blood or the blood border position and using the sheath as a zero point for measurements to reduce or remove error(s) or the effects caused by changing environmental materials and/or condition(s);
(ii) once the image or the catheter or probe is externally calibrated, inserting the catheter or probe into the target, object, or sample;
(iii) once the image or the catheter or probe is externally calibrated and the catheter or probe is inserted into the target, object, or sample, then keeping the delay line, or the motorized delay line, the same without movement, and correcting any calibration error by adjusting the image of the one or more images or A-line images; and/or
(iv) one or more of the following: (1) acquiring one image or A-line image of the one or more images or A-line images; (2) applying bilateral filtering and Otsu's thresholding method to one image or A-line image of the one or more images or A-line images; (3) detecting a bottom line area of a biggest detected object or binary object, where the bottom line area corresponds to an outer sheath boundary for the catheter or the probe; and/or (4) shifting one image or A-line image of the one or more images or A-line images such that a detected outer sheath boundary matches or substantially matches a zero point or position which corresponds to a ring mark or a mark of a set or predetermined size and shape or which corresponds to an outer surface of a sheath of the catheter or probe, where all distances are measured outward from the zero point or position.

23. The method of claim 14, wherein the apparatus further comprises:
an interference optical system that operates to: (i) receive and divide light from a light source into a first light with which the target, object, or sample is to be irradiated and which travels along a sample arm of the interference optical system and a second reference light, (ii) send the second reference light along a reference arm of the interference optical system for reflection off of a reference reflection of the interference optical system, and (iii) generate interference light by causing reflected or scattered light of the first light with which the target, object, or sample has been irradiated and the reflected second reference light to combine or recombine, and to interfere, with each other, the interference light generating one or more interference patterns; and
one or more detectors that operate to continuously acquire the interference light and/or the one or more interference patterns to measure the interference or the one or more interference patterns between the combined or recombined light to obtain data for one or more imaging modalities,
wherein: (i) a wavelength of the first light is shorter than a wavelength of the reflected or scattered light and/or the generated interference light, and/or (ii) the interference optical system or the catheter or probe includes a double clad fiber.

24. The method of claim 23, further comprising one or more of the following:
(i) using the light source that operates to produce the light;
(ii) using the light source that operates to produce the light, the light source producing the light to operate as an excitation laser or light having a wavelength of 400 nm-900 nm or 635 nm; and/or
(iii) using the light source that operates to produce the light, the light source producing the light as an excitation laser or light and coupling the excitation laser or light into the interference optical system, the optical probe, and/or one or more components of the optical probe and/or of the catheter.

25. The method of claim 14, further comprising one or more of the following:
(i) performing a pullback of the catheter or probe and/or obtaining or receiving the one or more images or the one or more A-line images of one or more imaging modalities from the pullback of the catheter or probe; and/or
(ii) displaying the one or more images or the one or more A-line images on a display, storing the one or more images or the one or more A-line images in a memory, or using the one or more images or the one or more A-line images to train one or more models or AI-networks to (a) perform the external calibration and/or the in vivo calibration and/or (b) automatically obtain the one or more images or the one or more A-line images of the one or more imaging modalities.

26. The method of claim 25, wherein, in a case where the method has trained one or more models or AI-networks, one or more of the following exists or occurs:
(i) the trained model is one or a combination of the following: a neural net model or neural network model, a deep convolutional neural network model, a recurrent neural network model with long short-term memory that can take temporal relationships across images or frames into account, a generative adversarial network (GAN) model, a consistent generative adversarial network (cGAN) model, a three cycle-consistent generative adversarial network (3cGAN) model, a model that can take temporal relationships across images or frames into account, a model that can take temporal relationships into account including tissue location(s) and/or calibration location(s) during pullback in a vessel and/or including tissue and/or calibration characterization data during pullback in a vessel, a model that can use prior knowledge about a procedure and incorporate the prior knowledge into the machine learning algorithm or a loss function, a model using feature pyramid(s) that can take different image resolutions into account, and/or a model using residual learning technique(s), a segmentation model, a segmentation model with post-processing, a model with pre-processing, a model with post-processing, a segmentation model with pre-processing, a deep learning or machine learning model, a semantic segmentation model or classification model, an object detection or regression model, an object detection or regression model with pre-processing or post-processing, a combination of a semantic segmentation model and an object detection or regression model, a model using repeated segmentation model technique(s), a model using feature pyramid(s), a genetic algorithm that operates to breed multiple models for improved performance, and/or a model using repeated object detection or regression model technique(s); and/or
(ii) the method further comprises using one or more neural networks or convolutional neural networks to one or more of: load a trained model of images or A-line images; perform external and/or in vivo calibration on the catheter or probe;
determine whether the external and/or in vivo calibration is/are accurate or correct; determine one or more of the characteristics of one or more objects, targets, or samples in the one or more images or the one or more A-line images; identify or detect the one or more objects, targets, or samples; overlay data on at least one of the one or more images or A-line images to show location(s) of intravascular image(s), the calibrated catheter or probe, and/or the objects, targets, or samples; incorporate image processing and machine learning (ML) or deep learning to automatically identify and locate portions or components of the catheter or probe and/or to perform external and/or in vivo calibration of the catheter or probe; incorporate image processing and machine learning (ML) or deep learning to automatically identify and locate the one or more objects, targets, or samples; display the results for the external and/or in vivo calibration, the identification/detection or characterization on a display; and/or acquire or receive image data during the pullback operation of the catheter or the optical probe.

27. The method of claim 14, wherein the one or more components of the catheter or probe include or comprise a double clad fiber.

28. A computer-readable storage medium storing at least one program that operates to cause one or more processors to execute a method for externally calibrating and in vivo calibrating a catheter or probe of an apparatus, the method comprising:
obtaining or receiving one or more images or one or more A-line images;
automatically calibrating the catheter or probe using an external calibration, before the catheter or probe is inserted into a target, sample, or object, by detecting one or more skeletons or portions of a sheath of the catheter or probe and determining whether the skeletons or portions of the sheath are in a target or set position; and
automatically calibrating the catheter or probe using an in vivo calibration, after the catheter or probe is inserted into the target, sample, or object, by detecting blood or a blood border position to automatically locate a position of the one or more skeletons or portions of the sheath and then adjusting or re-adjusting the image or the A-line image and calibrating or re-calibrating the catheter or probe to reduce or remove any effects caused by in vivo or environmental changes.
